(19) United States
(12) Patent Application Publication
(10) Pub. No.: US 2025/0037787 A1
(43) Pub. Date: Jan. 30, 2025

(54) CONCENTRATION BOUNDS IN LARGE NETWORKS

(71) Applicant: Max-Planck-Gesellschaft zur Förderung der Wissenschaften e.V., München (DE)

(72) Inventors: Anika Küken, Nuthetal (DE); Georg Basler, Berlin (DE); Jeanne Marie Onana Eloundou Mbebi, Potsdam (DE); Zoran Nikoloski, Berlin (DE)

(21) Appl. No.: 16/967,961

(22) PCT Filed: Feb. 12, 2019
(86) PCT No.: PCT/EP19/53384

§ 371 Date: Aug. 6, 2020

(30) Foreign Application Priority Data
Feb. 12, 2018 (EP) EP18156341.2

(51) Int. Cl. G16B 5/00; G06F 17/16

(52) U.S. Cl. CPC G16B 5/00; G06F 17/16

(57) ABSTRACT

The present invention relates to a computer-implemented method of calculating the ranges of concentrations, of fluxes, or of reaction rate constants in a network of chemical reactions.

The present invention relates to a computer-implemented method of calculating
(a) the ranges of the concentration xᵢ of a component Xᵢ;
(b) the flux(es) and/or flux ratio(s) which determine the concentration ranges of the said component Xᵢ; and/or
(c) the reaction rate constant(s) and/or their ratio(s) which determine the concentration ranges of said component Xᵢ in a network of chemical reactions defined by a stoichiometric matrix N;
said calculating comprising evaluating formula (1):

$$min \{ Q , F , {P}_{j} \} {λ}_{i} \leq {x}_{i} \leq max \{ Q , F , {P}_{j} \} {λ}_{i} \qquad ( 1 )$$

wherein
(i) Nis N⁺-N⁻;
   N⁺ is the matrix defining the stoichiometry of products of each reaction in said network;
   N⁻ is the matrix defining the stoichiometry of substrates of each reaction in said network;
   Sⱼ is the set of reactions in said network which have a component Xⱼ as one of their substrates;
   F is a set of steady-state fluxes; and
   Pⱼ is the set of reactions in said network which have component Xⱼ as one of their products;

$$\begin{matrix}( ii ) & \\ {λ}_{i} = \frac{{σ}_{p}}{{σ}_{s}^{- i}} \frac{{v}_{p}}{{v}_{s}^{- i}} ; & \end{matrix}$$

vₚ is the flux of reaction Rₚ∈Pⱼ, Rₚ having Xⱼ as one of its products;
vₛ⁻ⁱ is the flux of reaction Rₛ^{-j}∈Q, Rₛ⁻ⁱ differing from reaction Rₛ in that one substrate molecule of Xᵢ is missing in comparison to reaction Rₛ.

$${σ}_{p} = {\sum }_{k ϵ {P}_{j}} {N}_{jk}^{+} \frac{{v}_{k}}{{v}_{p}} ;$$

$${σ}_{s}^{- i} = \text{?} {N}_{jl}^{-} \frac{{θ}_{l}}{{θ}_{l}^{- i}} \frac{{v}_{l}^{- i}}{{v}_{s}^{- i}} ;$$

$$\text{?} \text{indicates text missing or illegible when filed}$$

θₗ is the reaction rate constant for reaction Rₗ;
θₗ⁻ⁱ is the reaction rate constant for reaction Rₗ⁻ⁱ;
(iii) Q is a subset of Sⱼ⁻ⁱ such that for every reaction Rₗ∈Sⱼ there is one reaction Rₗ⁻ⁱ∈Q; and
   Sⱼ⁻ⁱ is a set of chemical reactions differing from the set of reactions Sⱼ in that one substrate molecule of Xᵢ is missing in comparison to a reaction R in Sⱼ.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Advances in systems biology studies have been propelled by the availability of high-quality genome-scale metabolic reconstructions for many organisms across all kingdoms of life (Bordbar, A., Monk, J. M., King, Z. A. & Palsson, B. O. Constraint-based models predict metabolic and associated cellular functions. Nature Reviews Genetics 15, 107-120, doi: 10.1038/nrg3643 (2014)). Metabolic network reconstructions contain information about metabolites and reactions through which they are transformed to support different cellular processes (Schuetz, R., Zamboni, N., Zampieri, M., Heinemann, M. & Sauer, U. Multidimensional optimality of microbial metabolism. Science 336, 601-604, doi: 10.1126/science.1216882 (2012); Schellenberger, J. et al. Quantitative prediction of cellular metabolism with constraint-based models: the COBRA Toolbox v2.0. Nature protocols 6, 1290-1307, doi: 10.1038/nprot.2011.308 (2011)). Alongside enzyme concentrations and phenomenological constants, reaction rates and metabolite concentrations—as two faces of the metabolic phenotype—characterize key aspects of the metabolic capability of an organism. Since metabolic concentrations are important determinants of reaction rates (Hackett, S. R. et al. Systems-level analysis of mechanisms regulating yeast metabolic flux. Science 354, doi: 10.1126/science.aaf2786 (2016)), understanding what controls their physiological ranges can point to cellular mechanisms of phenotypic robustness that ensures viability of organisms under changing conditions (Kitano, H. Towards a theory of biological robustness. Molecular systems biology 3, 137. doi: 10.1038/msb4100179 (2007)).

The change in concentration of metabolites, or, more generally, of components, can be described by a system of coupled ordinary differential equations (ODEs),

$$\frac{dx ( t )}{dt} = Nv ( t ) ,$$

where v(t)=(v₁(t), . . . , vₙ(t))^{T} denotes reaction rates and x(t)=(x₁(t), . . . , xₘ(t)) the component concentrations at time t, and N represents the stoichiometric matrix. The rows of the stoichiometric matrix correspond to components, columns stand for reactions, and its entries denote the stoichiometric coefficients with which components participate in reactions as substrates or products. Reaction rates are modeled according to a kinetic law, v(t)=f(x(t),θ), which often leads to nonlinearities and involves multiple parameters, denoted by θ (Heinrich, R. & Schuster, S. The Regulation of Cellular Systems. 1 edn, (Springer US, 1996)). As a result, the coupled nonlinear ODEs are often not analytically tractable and their simulations are challenging. These issues arise since parameters remain poorly specified at a genome scale for the majority of model organisms (Khodayari, A., Zomorrodi, A. R., Liao, J. C. & Maranas, C. D. A kinetic model of Escherichia coli core metabolism satisfying multiple sets of mutant flux data. Metabolic engineering 25, 50-62, doi: 10.1016/j.ymben.2014.05.014 (2014); Davidi, D. et al. Global characterization of in vivo enzyme catalytic rates and their correspondence to in vitro k_{cat} measurements. Proceedings of the National Academy of Sciences of the United States of America 113, 3401-3406, doi: 10.1073/pnas.1514240113 (2016)) and the nonlinear ODEs may lead to numerical issues (Press, W. H. Numerical recipes in C: the art of scientific computing. (Cambridge University Press, 1988)).

Feasible steady-state reaction rates, v, for which Nv=0, can be predicted based solely on the structure of the network with computational approaches from the constraint based modeling framework (Lewis, N. E., Nagarajan, H. & Palsson, B. O. Constraining the metabolic genotype-phenotype relationship using a phylogeny of in silico methods. Nature reviews. Microbiology 10, 291-305, doi: 10.1038/nrmicro2737 (2012)). However, since intracellular reaction rates cannot be measured directly, the validation of these predictions requires laborious labeling experiments and model fitting procedures (Niedenfuhr, S., Wiechert, W. & Noh, K. How to measure metabolic fluxes: a taxonomic guide for (13) C fluxomics. Current opinion in biotechnology 34, 82-90, doi: 10.1016/j.copbio.2014.12.003 (2015)). By neglecting the effect of concentrations on reaction rates, constraint based approaches do not facilitate the usage of network reconstructions to predict concentrations of components or metabolites, which are readily accessible by metabolomics techniques (Johnson, C. H., Ivanisevic, J. & Siuzdak, G. Metabolomics: beyond biomarkers and towards mechanisms. Nature reviews. Molecular cell biology 17, 451-459, doi: 10.1038/nrm.2016.25 (2016)). Therefore, a method to predict component concentration ranges with limited knowledge about the underlying kinetic laws and parameter values would allow direct integration and validation of genome-scale models with experimental data, enabling systems biology applications, from engineering of intervention strategies to design of new drugs (Shaked, I., Oberhardt, M. A., Atias, N., Sharan, R. & Ruppin, E. Metabolic Network Prediction of Drug Side Effects. Cell systems 2, 209-213, doi: 10.1016/j.cels.2016.03.001 (2016); Pharkya, P., Burgard, A. P. & Maranas, C. D. OptStrain: a computational framework for redesign of microbial production systems. Genome research 14, 2367-2376, doi: 10.1101/gr.2872004 (2004); Ranganathan, S., Suthers, P. F. & Maranas, C. D. OptForce: an optimization procedure for identifying all genetic manipulations leading to targeted overproductions. PLoS computational biology 6, e1000744, doi: 10.1371/journal.pcbi.1000744 (2010)).

The search for structural determinants of concentration ranges has prompted the development of a rich mathematical theory to determine network components exhibiting the same steady-state concentration irrespective of the changes in the environment (Karp, R. L., Perez Millan, M., Dasgupta, T., Dickenstein, A. & Gunawardena, J. Complex-linear invariants of biochemical networks. Journal of theoretical biology 311, 130-138, doi: 10.1016/j.jtbi.2012.07.004 (2012); Dexter, J. P. & Gunawardena, J. Dimerization and bifunctionality confer robustness to the isocitrate dehydrogenase regulatory system in Escherichia coli. The Journal of biological chemistry 288, 5770-5778, doi: 10.1074/jbc.M112.339226 (2013); Shinar, G. & Feinberg, M. Structural sources of robustness in biochemical reaction networks. Science 327, 1389-1391, doi: 10.1126/science.1183372 (2010); Feinberg, M. Chemical reaction network structure and the stability of complex isothermal reactors—I. The deficiency zero and deficiency one theorems. Chemical Engineering Science 42, 2229-2268 (1987)). However, the identified structural determinants underlying this qualitative concentration-related property do not hold for large-scale networks, limiting the applicability of the elegant results (Eloundou-Mbebi, J. M. et al. A network property necessary for concentration robustness. Nature communications 7, 13255, doi: 10.1038/ncomms13255 (2016)). In addition, determining the steady-state concentration ranges by characterizing the solutions to the system of non-linear equations Nf(x(t),θ)=0 is intractable for large-scale networks even when the equations have a simplified form often used in metabolic modeling (Cox, D. A., Little, J. & O'Shea, D. Ideals, Varieties, and Algorithms—An Introduction to Computational Algebraic Geometry and Commutative Algebra. 3 edn, (Springer-Verlag New York, 2007)).

In view of the deficiencies of the prior art, the technical problem underlying the present invention can be seen in the provision of means and methods of calculating concentration ranges in complex networks of chemical, including biochemical, reactions.

The technical problem is solved by the subject-matter of the claims.

Accordingly, in a first aspect, the present invention relates to a computer-implemented method of calculating
(a) the ranges of the concentration xᵢ of a component Xᵢ;
(b) the flux(es) and/or flux ratio(s) which determine the concentration ranges of the said component Xᵢ; and/or
(c) the reaction rate constant(s) and/or their ratio(s) which determine the concentration ranges of said component Xᵢ in a network of chemical reactions defined by a stoichiometric matrix N;
said calculating comprising evaluating formula (1):

$$min \{ Q , F , {P}_{j} \} {λ}_{i} \leq {x}_{i} \leq max \{ Q , F , {P}_{j} \} {λ}_{i} \qquad ( 1 )$$

wherein
(i) N is N⁺-N⁻;
   N⁺ is the matrix defining the stoichiometry of products of each reaction in said network;
   N⁻ is the matrix defining the stoichiometry of substrates of each reaction in said network;
   Sⱼ is the set of reactions in said network which have a component Xⱼ as one of their substrates;
   F is a set of steady-state fluxes; and
   Pⱼ is the set of reactions in said network which have component Xⱼ as one of their products;

$$\begin{matrix}( ii ) & \\ {λ}_{i} = \frac{{σ}_{p}}{{σ}_{s}^{- i}} \frac{{v}_{p}}{{v}_{s}^{- i}} ; & \end{matrix}$$

vₚ is the flux of reaction Rₚ∈Pⱼ, Rₚ having Xⱼ as one of its products;
vₛ⁻ⁱ is the flux of reaction Rₛ⁻ⁱ∈Q, Rₛ⁻ⁱ differing from reaction Rₛ in that one substrate molecule of Xᵢ is missing in comparison to reaction Rₛ.

$${σ}_{p} = {\sum }_{k ∈ {P}_{j}} {N}_{jk}^{+} \frac{{v}_{k}}{{v}_{p}} ;$$

$${σ}_{s}^{- i} {\sum }_{l ∈ {S}_{j}} {N}_{ji}^{-} \frac{{θ}_{l}}{{θ}_{l}^{- i}} \frac{{v}_{l}^{- i}}{{v}_{s}^{- i}} ;$$

θₗ is the reaction rate constant for reaction Rₗ;
θₗ⁻ⁱ is the reaction rate constant for reaction Rₗ⁻ⁱ;
(iii) Q is a subset of Sⱼ⁻ⁱ such that for every reaction Rᵢ∈Sⱼ there is one reaction Rₗ⁻ⁱ∈Q; and
   Sₗ⁻ⁱ is a set of chemical reactions differing from the set of reactions Sⱼ in that one substrate molecule of Xᵢ is missing in comparison to a reaction R in Sⱼ.

In accordance with the invention, calculating is effected on a computer. Further aspects of the invention, in particular a computer program and a computer-readable medium illustrate the computer-based nature of the method in accordance with the first aspect.

The process of “calculating” the recited concentrations, fluxes and reaction rate constants, respectively, can also be termed “predicting” these parameters.

On the other hand, “calculating” has to be held distinct from simulating. Deviant from prior art approaches which employ simulation, it is a hallmark of the present invention to provide simulation-free prediction of concentration ranges.

The term “component” means any chemical compound including biomolecules, macromolecules, metabolites and small molecules. Preferred components are defined further below. The components satisfying the equation of formula (1) are said to have structurally constrained concentrations.

As is apparent from formula (1) as given above, lower and upper bounds for the concentration of a given component is determined by the method of the invention. It is understood that in particular in those instances where the difference between the lower and the upper bound is small or even negligible, the method of calculating in accordance with the present invention effectively provides concentrations.

Said lower and upper bounds are obtained as extreme values (minimum and maximum) of the parameter λ, which parameter is defined in part (ii) of the definitions section of formula (1).

Generally speaking, concentrations, fluxes (also referred to as “reaction rates” herein) and rate constants (also referred to as “reaction rate constants”) are interrelated. A generic expression of the underlying kinetic law is given in the background section above. Owing to the kinetic law, knowledge of two of the parameters allows, roughly speaking, calculation of the third. As will become apparent in more detail further below, it surprisingly turns out that a complete knowledge of two parameters in a network is not a prerequisite to calculate the third parameter. As shown in the examples, in a case where only 30% of the relevant rate constants were known, concentrations could be satisfactorily calculated. As explained in more detail below, the rate constants which appear in the expression for σₛ⁻ⁱ and σₚ for any Q⊆Sⱼ⁻ⁱ are referred to as relevant rate constants.

A key feature of the invention is the approach chosen to calculate lower and upper bounds for the concentration of the given component of the network under consideration. In order to determine these extrema, all possible sets Q of chemical reactions in a given network are considered, and furthermore all flux distributions F, and all reactions in the set of reactions designated Pⱼ. As such, both Q and Pⱼ designate a set of reactions, said set of reactions being as defined herein above.

As noted above, reactions Rₛ⁻ⁱ and Rₛ differ from each other in that in Rₛ⁻ⁱ one molecule of Xᵢ is missing. To give an example, if Rₛ is second order with regard to Xᵢ, then Rₛ⁻ⁱ is first order with regard to Xᵢ. If Rₛ is first order with regard to Xᵢ, then Xᵢ is absent in Rₛ⁻ⁱ.

Preferably, the method of the invention is applied to components with structurally constrained concentrations (SCCs) as well as for components revealed to have SCCs upon application of an extended approach (see below).

A derivation of formula (1), which is based on three conditions (i) to (iii) as developed in the course of the present invention is given below. In brief, in accordance with the invention, conditions are derived that establish a direct link between the structure of a (bio) chemical network, ratios of relevant rate constants, and ratios of selected reaction fluxes on one side, and quantitative concentration ranges of particular components on the other. According to the invention, this link is based on the concept of full coupling of reactions which is expanded under the assumption of mass action kinetics to include reactions that share substrates of same stoichiometry.

The method of the invention allows to efficiently determine concentration ranges in large-scale networks such as chemical and biochemical networks endowed with mass action kinetics. The same conditions facilitate the identification of components that exhibit absolute concentration robustness in genome-scale networks, which is not possible with the existing approaches. Therefore, the present invention allows identifying components with structurally constrained concentrations and absolute concentration robustness in large-scale networks across all kingdoms of life.

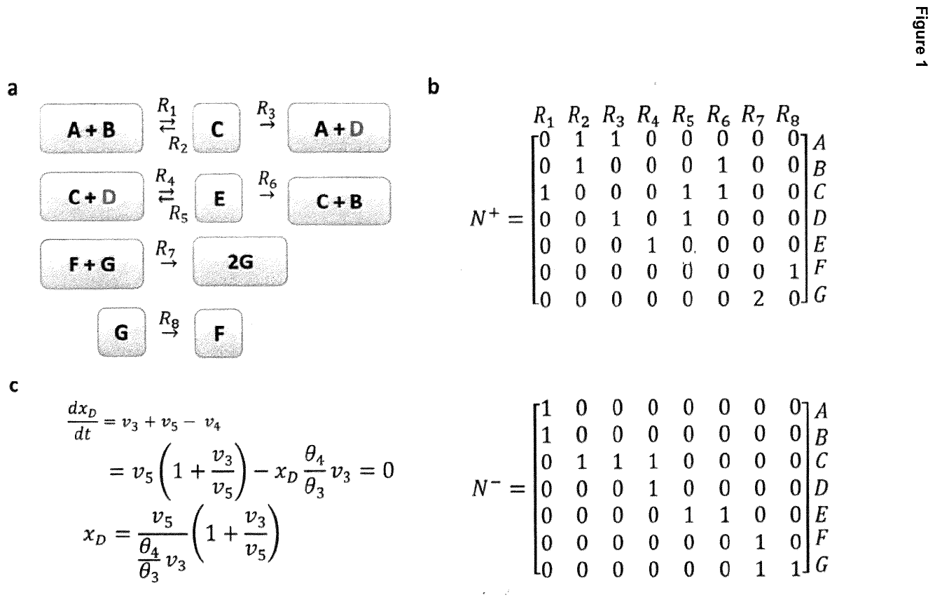

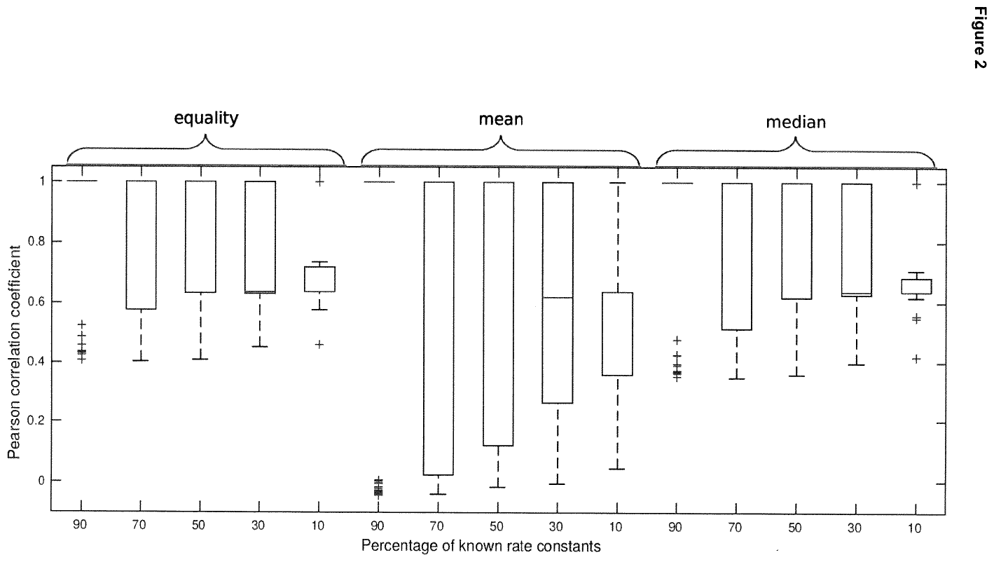

Consider a network composed of m components that participate in n reactions. The (m×n) stoichiometric matrix, N, can be written as a difference of two non-negative matrices, N=N⁺−N⁻, where N⁺ includes the stoichiometry of the products and N⁻ comprises the stoichiometry of the substrates of each reaction. For instance, the stoichiometry of substrates and products given in FIG. 1b describes the network on FIG. 1a. We assume that the rate of reaction Rᵢ is modeled according to mass action kinetic, whereby

$${v}_{i} = {θ}_{i} {Π}_{j} {x}_{j}^{{N}_{ji}^{-}} ,$$

where θᵢ>0 is the reaction constant and the concentration xⱼ of each substrate molecule j appears in vᵢ as a multiplicative factor.

In the following, concepts and terminology used herein are introduced. We will say that a reaction Rₖ lacks one substrate molecule of Xᵢ in comparison to reaction Rₗ, if Nᵢₗ⁻−Nᵢₖ⁻=1 and for every i′≠i, N_{i,l}⁻−N_{i/k}⁻=0. For the network in FIG. 1a, reaction R₃ lacks one substrate molecule of component D in comparison to reaction R₄, and reaction R₈ lacks one substrate molecule of component F in comparison to reaction R₇. Under the assumption of mass action kinetic, if a reaction lacks one substrate molecule in comparison to another, the reactions differ in their orders by one. As a result, the ratio of fluxes for such reactions depends only on the rate constants and the concentration of the substrate in which the reactions differ.

Furthermore, two reactions Rₖ and Rᵢ are fully coupled if there exists λ>0, such that vᵢ=λvₖ for any positive steady-state reaction rate v. i.e., Nv=0 (Burgard, A. P., Nikolaev, E. V., Schilling, C. H. & Maranas, C. D. Flux coupling analysis of genome-scale metabolic network reconstructions. Genome research 14, 301-312, doi: 10.1101/gr.1926504 (2004)). Therefore, fully coupled reactions have an invariant ratio 2 over all positive steady states that the network admits, and full coupling is a transitive relation. For the network in FIG. 1a, reactions R₆ and R₃ are fully coupled, since the intermediate components D and E can only be maintained at a constant concentration if the fluxes of both R₆ and R₃ equal the net flux v₄-v₅. Such reactions, which are fully coupled irrespective of the kinetic law, can be efficiently determined based on the stoichiometry of large-scale networks by linear programming (Burgard, A. P., Nikolaev, E. V., Schilling, C. H. & Maranas, C. D. Flux coupling analysis of genome-scale metabolic network reconstructions. Genome research 14, 301-312, doi: 10.1101/gr.1926504 (2004); Larhlimi, A., David, L., Selbig, J. & Bockmayr, A. F2C2: a fast tool for the computation of flux coupling in genome-scale metabolic networks. BMC bioinformatics 13, 57, doi: 10.1186/1471-2105-13-57 (2012)) (for details see Example 1, section entitled “Flux coupling”).

Under the assumption of mass action kinetic, two reactions that share the same substrates of same stoichiometry are also fully coupled (Neigenfind, J., Grimbs, S. & Nikoloski, Z. On the relation between reactions and complexes of (bio) chemical reaction networks. Journal of theoretical biology 317, 359-365, doi: 10.1016/j.jtbi.2012.10.016 (2013)). In this case, the coupling holds for any, not necessarily steady, state of the system. Therefore, the consideration of mass action kinetic expands the set of fully coupled reactions. For instance, this is the case for reactions R₅ and R₆ that have the same substrate component E in FIG. 1a, whereby

$$\frac{{v}_{5}}{{v}_{6}} = \frac{{θ}_{5}}{{θ}_{6}} .$$

Since R₆ and R₃ are fully coupled and the relation of full coupling is transitive, the reactions R₅ and R₃ are also fully coupled.

Consider now a component Xⱼ with an ordinary differential equation (ODE) given by

$$\frac{{dx}_{j}}{dt} = {\sum }_{k ∈ {P}_{j}} {N}_{jk}^{+} {v}_{k} - {\sum }_{l ∈ {S}_{j}} {N}_{ji}^{-} {v}_{l} ,$$

where Pⱼ is the set of reactions with Xⱼ as one of their products and Sⱼ is the set of reactions which have component Xⱼ as one of their substrates. A component Xᵢ, not necessarily different from Xⱼ, has structurally constrained concentration (SCC), if the following conditions hold:
(i) for each reaction R in Sⱼ, there is at least one reaction in the network which lacks one molecule of Xᵢ in comparison to R, yielding the set of reactions Sⱼ⁻ⁱ;
(ii) all reactions in Sⱼ⁻ⁱ are mutually fully coupled; and
(iii) all reactions in Pⱼ are mutually fully coupled.

A similar derivation can be made if in condition (i) for each reaction in Pⱼ, one can identify a reaction which lacks one molecule of Xᵢ (see Example 2).

In the following, we use the ODE for component Xⱼ to derive the concentration bounds for a component Xᵢ with SCC. Let Q be a subset of Sⱼ⁻ⁱ such that for every reaction Rₜ∈Sⱼ there is one reaction Rₜ⁻ⁱ∈Q. Under mass action, for the flux of every reaction Rₜ∈Sⱼ, it holds that

$${v}_{l} = {x}_{i} \frac{{θ}_{l}}{{θ}_{l}^{- i}} {v}_{l}^{- i}$$

(see Example 2), where θₗ⁻ⁱ is the reaction constant and vₗ⁻ⁱ the flux of reaction

Rₗ⁻ⁱ∈Q. The expression for

$$\frac{{dx}_{j}}{dt}$$

above then becomes

$${\sum }_{k ∈ {P}_{j}} {N}_{jk}^{+} {v}_{k} - {x}_{i} {\sum }_{l ∈ {S}_{j}} {N}_{ji}^{-} \frac{{θ}_{l}}{{θ}_{l}^{- i}} {v}_{l}^{- i} .$$

At any positive steady state, it then holds that

$$\frac{{dx}_{j}}{dt} = {v}_{p} {\sum }_{k ∈ {P}_{j}} {N}_{jk}^{+} \frac{{v}_{k}}{{v}_{p}} - {x}_{i} {v}_{s}^{- i} {\sum }_{l ∈ {S}_{j}} {N}_{jl}^{-} \frac{{θ}_{l}}{{θ}_{l}^{- i}} \frac{{v}_{l}^{- i}}{{v}_{s}^{- i}} = 0 ,$$

for any flux vₚ of reaction Rₚ∈Pⱼ and flux vₛ⁻ⁱ of reaction Rₛ⁻ⁱ∈Q. Due to the conditions (iii), above, the sum

$${σ}_{p} = {\sum }_{k ∈ {P}_{j}} {N}_{jk}^{+} \frac{{v}_{k}}{{v}_{p}}$$

is a constant winch, in the simplest case, when all reactions in Pⱼ are fully coupled irrespective of the kinetic rate law, depends only on the network structure. In addition, due to condition (ii), above, the value of

$${σ}_{s}^{- i} = {\sum }_{l ∈ {S}_{j}} {N}_{ji}^{-} \frac{{θ}_{l}}{{θ}_{l}^{- i}} \frac{{v}_{l}^{- i}}{{v}_{s}^{- i}}$$

is also a constant which depends on both the network structure and a subset of rate constants. The rate constants which appear in the expression for σₛ⁻ⁱ and σₚ for any Q⊆Sⱼ⁻ⁱ will be referred to as relevant rate constants.

Therefore, given a steady-state flux distribution, v, a set Q⊆Sⱼ⁻ⁱ, and two reactions Rₚ∈Pⱼ and Rₛ⁻ⁱ∈Q, we have that

$${x}_{i} = \frac{{σ}_{p}}{{σ}_{s}^{- i}} \frac{{v}_{p}}{{v}_{s}^{- i}} .$$

The concentration bounds for xᵢ over any set, F, of steady-state flux distributions, any subset Q, and reactions in Pⱼ are then given as:

$${min}_{( Q , F , {P}_{j} \}} \frac{{σ}_{p}}{{σ}_{s}^{- i}} \frac{{v}_{p}}{{v}_{s}^{- i}} \leq {x}_{i} \leq {max}_{( Q , F , {P}_{j} \}} \frac{{σ}_{p}}{{σ}_{s}^{- i}} \frac{{v}_{p}}{{v}_{s}^{- i}} \qquad ( 1 a )$$

For instance, due to the full coupling of reactions R₃ and R₅ in FIG. 1a, component D exhibits structurally constrained concentration that depends only on rate constants (FIG. 1c).

Formula (1a) as given above is an alternative representation of formula (1) as recited in relation to the first aspect of the invention above.

The method in accordance with the first aspect of the invention is advantageously characterized in that it is applicable to large networks. Art-established methods, as reviewed in the background section herein above, are limited to networks of considerably smaller size. Furthermore, the method of the present invention does not rely on simulating reactions and fluxes, but instead calculates concentrations or, in the alternative, fluxes, or, in another alternative, rate constants.

In terms of input data, the method of the present invention does not present a requirement for cumbersome experiments involving isotope labeling to determine fluxes. This is a further advantageous distinction from the art-established methods for flux estimation from experiments.

Furthermore, it has to be noted that art-established methods, also known as “constraint-based approaches” can only predict fluxes. They are not capable of predicting bounds on concentrations. The method in accordance with the first aspect, however, does deliver bounds on concentrations.

The method, in one embodiment, relies on the availability of information about relevant rate constants to estimate concentration ranges. Therefore, there is a need to investigate the effect of having only partial information about the relevant rate constants on the predicted concentration ranges. To this end, it is demonstrated that even when only 30% of relevant rate constants are known (similar to the current state for a large-scale network of E. coli), there is a good qualitative agreement between predicted and simulated concentration ranges.

In a preferred embodiment (a) concentrations and/or ranges thereof are determined using fluxes and reaction rate constants as input; (b) fluxes, flux ratios and/or ranges thereof are determined using concentrations and reaction rate constants as input; or (c) reaction rate constants, their ratios and/or ranges thereof are determined using concentrations and fluxes as input.
Item (a) of this preferred embodiment provides concentrations and/or ranges thereof as an output of the calculation. The other two parameters of the kinetic law, fluxes and reaction rate constants, are used as input. As noted above, prior art methods are not capable of calculating concentrations in a network.

Rate constants can be measured and/or obtained from the literature. Fluxes can be obtained, for example, as described in Niedenfuhr et al., loc. cit. Fluxes can also be obtained by constraint-based approaches by imposing bounds on the exchange fluxes, which can be measured, and/or optimizing relevant objectives (e.g., biomass, ATP usage).
Item (b) relates to the calculation of fluxes. For that purpose, concentrations have to be known or estimated.
Item (c) provides for the calculation of rate constants.

In a further preferred embodiment (a) up to 10%, up to 20%, up to 30%, up to 40%, up to 50%, up to 60%, up to 70% or up to 80% of said input is estimated; (b) experimentally determined input does not require experiments involving isotope labelling; and/or (c) concentrations, to the extent they are used as input, are determined by means of mass spectrometry.

Estimated input in accordance with item (a) may be estimated concentrations. Such estimates may be global mean of the concentration for the respective component. “Global mean” refers to the mean value over a set of experiments.

A preferred method of determining amounts or concentrations of components Xᵢ is mass spectrometry.

In a further preferred embodiment said method is applied two or more times, and wherein each time more input is provided, the additional input being derived from one or more previous runs. To explain further, provided with experimental measurements of component concentrations, the concentrations of components with SCC can be fixed to the measured values. By formula (1), this fixes the ratio of the two fluxes appearing in the formula. Conditions (i)-(iii), above, can then be checked for additional components with the constraints on flux ratios.

The method can be further generalized to the following three conditions: (i) if only for a subset G of reaction R in Sⱼ, there is at least one reaction in the network which lacks one molecule of Xᵢ in comparison to R, yielding the set of reactions Sⱼ⁻ⁱ (G), (ii) all reactions in Sⱼ⁻ⁱ (G) are fully coupled and (iii) all reactions in Pj and Sⱼ−G are fully coupled and their contribution is the ODE is positive over the steady-state fluxes in F. If these three conditions hold, then formula (1) also holds. This generalization of the method is herein also referred to as “extended approach”.

In a further preferred embodiment, said method is applied (a) to at least two networks with different architectures, said different architectures being defined in terms of different stoichiometric matrices N; and/or (b) to the same network using at least two different inputs.
Item (a) relates to applications which allow to compare different networks, in particular how the parameters concentration, flux and reaction rate constant differ (or do not differ) across networks. To give examples, different networks may correspond to different cells, different organs and/or different species.
Item (b) provides for analyzing how a given network responds to different input parameters.

In a further preferred embodiment, the method of the invention furthermore comprises identifying for a given network and given inputs those components for which the ratio defined by formula (2)

$$\frac{max \{ Q , F , {P}_{j} \} {x}_{i}}{min \{ Q , F , {P}_{j} \} {x}_{i}} \qquad ( 2 )$$

is less than 2.0, less than 1.5, less than 1.2, less than 1.1, less than 1.05 or equal to 1.0, thereby identifying components exhibiting absolute concentration robustness (ACR).

This preferred embodiment introduces the notion of absolute concentration robustness (ACR). Components of a network which exhibit absolute concentration robustness are those components which are subject to only minor concentration changes in response to different conditions, conditions being specified by Q. F and Pⱼ.

Presence of absence of absolute concentration robustness for a given component is indicative of the fluctuation bandwidth of the concentration xᵢ of a component Xᵢ. Similarly, the closer the value of the ratio of formula (2) is to 1.0, the smaller is the fluctuation bandwidth of said concentration xᵢ.

In relation to fluctuation range, the present invention introduces a novel concept of a marker. As established in the art, a marker allows to distinguish between two different states of a system. Typically, markers which are deemed useful are those which exhibit a statistically significant difference between the mean concentration in a first state and the mean concentration in a second state.

Deviant therefrom or in addition thereto, the present invention introduces the notion of a marker, wherein said marker is characterized in that it has statistically significantly different fluctuation ranges in different states of a system. This notion of a marker does not require (but does not exclude) statistically significant differences between means.

Using the terminology of the present invention, such markers can be used to distinguish both between networks and between two different states of a given network, said different states of a given network arising from different inputs. These principles are laid down in the following preferred embodiment.

In a further preferred embodiment, a fine-tuned method of calculating of the first aspect uses formula (3) which is derived as follows. Let the lower and upper bounds for the concentration of metabolite Xᵢ derived from the ODE of metabolite Xⱼ in Formula (1) be denoted by

$${L}_{i}^{j} = {min}_{( Q , F \}} \frac{{σ}_{p}}{{σ}_{s}^{- i}} \frac{{v}_{p}}{{v}_{s}^{- i}}$$

$$and$$

$${U}_{i}^{j} = {max}_{( Q , F \}} \frac{{σ}_{p}}{{σ}_{s}^{- i}} \frac{{v}_{p}}{{v}_{s}^{- i}} ,$$

respectively. If there are r metabolites X_{d}, 1≤d≤r for which Eq. (1) applies, then the lower and upper bounds for the concentration of Xᵢ are given by the intersection of the ranges derived from the ODEs of X_{d}, i.e.

$${max}_{d} {L}_{i}^{d} \leq {x}_{i} \leq {min}_{d} {U}_{i}^{d} . \qquad ( 3 )$$

Therefore, the lower bound is the minimum of the maxima, while the upper bound is the maximum of the minima derived from the individual ODEs. In case that the SCC of a metabolite can be derived from multiple ODEs, Formula (3) provides more constrained predictions about metabolite concentration ranges than Formula (1) alone.

We note that the terms “equation” and “formula” are used equivalently.

In a further preferred embodiment of the method of the first aspect, (a) to the extent at least two networks with different networks are considered, furthermore comprises identifying those components which exhibit ACR in a first given network, but not in a second given network, thereby identifying a marker which allows to distinguish said first network from said second network; or (b) to the extent at least two different inputs for the same network are considered, further comprises identifying those components which exhibit ACR for a first given input, but not for a second given input, thereby identifying a marker which allows to distinguish a first state of said network from a second state of said network.

Given that the method of the present invention delivers concentrations or concentration ranges, respectively, it is also applicable for the purpose of determining markers in a conventional sense, namely components of a network with statistically significant different means in different states or different networks.

Accordingly, in a further preferred embodiment, the method of the first aspect of the invention further comprises determining concentration range or mean concentration for Xᵢ in each network or for each input, wherein statistically significant differences between the concentration ranges or mean concentrations for a first given network and a second given network are indicative of component Xᵢ being a marker which allows to distinguish a first network from a second network, respectively, and statistically significant differences between the concentration ranges or mean concentrations for a first given input and a second given input are indicative of component X; being a marker which allows to distinguish a first network state from a second network state.

Given that the term “network” comprises chemical networks, but also extends to biochemical networks, e.g. networks as they are found in cells or living organisms, it follows that in a further preferred embodiment said first network or network state is a healthy organism or cell or a healthy state of an organism or cell, respectively, and said second network or network state is a diseased organism or cell or a diseased state of an organism or cell, respectively.

In an agricultural setting, having networks for two environments (e.g., drought as first network or network state and ambient as second network or network state), the method allows the detection of biomarkers that distinguish between the two environments.

Analogously, in a further preferred embodiment said first network or network state is a wild-type organism or cell, and said second network or network state is a mutant organism or cell.

In a further preferred embodiment, said network (a) is cell-wide, organism-wide, cell-free or chemical network; (b) comprises more than 50, more than 100, or more than 200 reactions; and/or (c) comprises second and/or higher order reactions.

As noted in the introductory section herein above, art-established methods are generally limited to small networks (apart from the further deficiency that they are not capable of delivering predictions for concentrations or ranges thereof). A further feature of chemical or biochemical reactions which renders their treatment by art-established methods complicated is the presence of second and/or higher order reactions. Higher order reactions can easily be treated with the method of the first aspect of the invention.

In a further preferred embodiment, said component is selected from proteins, polypeptides, nucleic acids, lipids, carbohydrates, small organic molecules, metabolites and any combination thereof. Exemplary metabolites are described in the examples and the figures.

This preferred embodiment illustrates the applicability of the present invention to biochemical networks as they occur in biological systems. The enumeration is exemplary and refers to the well-known major classes of biomolecules.

In a second aspect, the present invention relates to use of formula (1) as defined in claim 1 for calculating the ranges of the concentration xᵢ of a component Xᵢ, the flux(es) and/or flux ratio(s) which determine the concentration ranges of the said component Xᵢ, and/or the reaction rate constant(s) and/or their ratio(s) which determine the concentration ranges of said component Xᵢ in a network of chemical reactions R defined by a stoichiometric matrix N.

Preferred embodiments of the method of the first aspect, to the extent applicable, define mutatis mutandis preferred embodiments of the use in accordance with the second aspect.

In a third aspect, the present invention provides a computer program comprising instructions to cause a computer to execute the steps of the method in accordance with the first aspect.

In a fourth aspect, the present invention provides a computer-readable medium (a) comprising instructions which, when executed on a computer, cause said computer to execute the steps of the method in accordance with the first aspect; and/or (b) having stored thereon the computer program in accordance with the third aspect.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

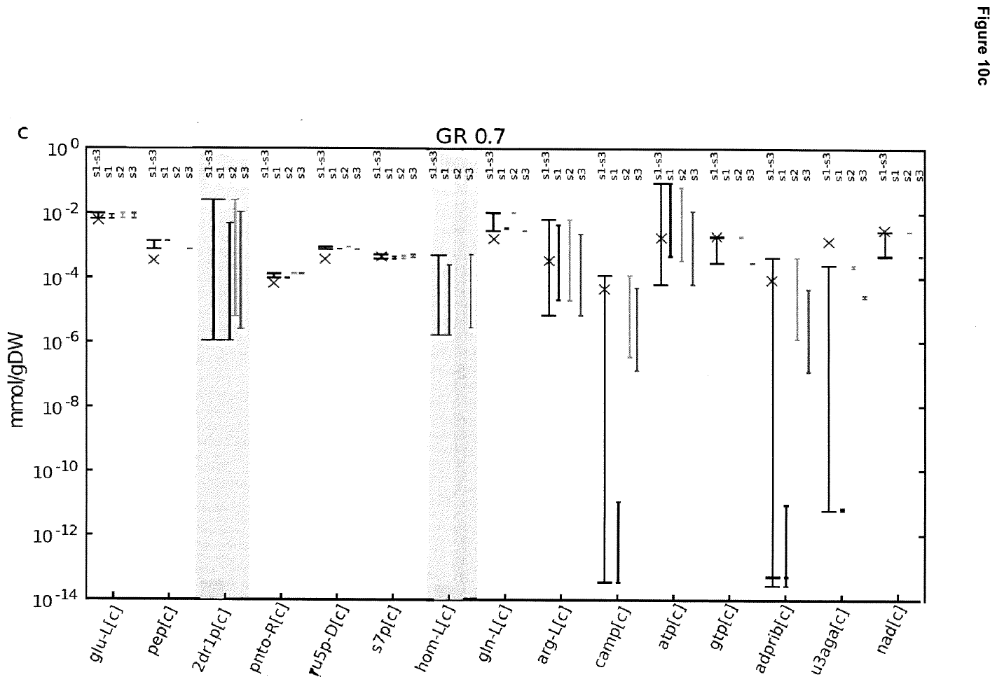

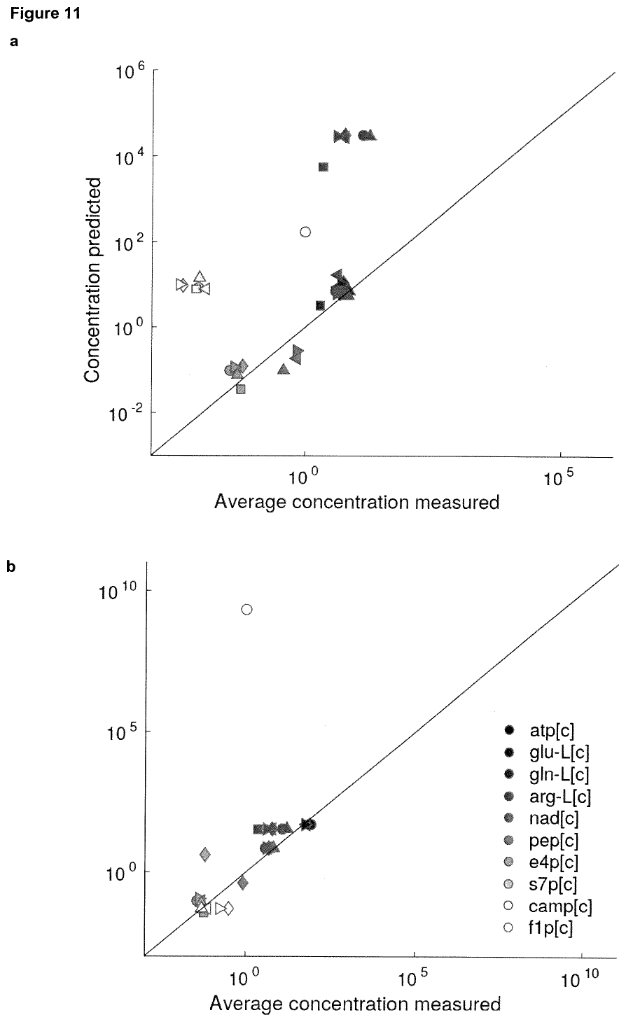

The figures show. [Please provide FIGS. 10 and 11 in a version which can be reproduced in black and white without losing information.]

FIG. 1: Network with components exhibiting structurally constrained concentration. (a) Reaction diagram that includes eight reactions, R₁-R₈, and seven components, A-G. (b) stoichiometric matrices associated with substrates, N⁻, and products, N⁺, for the network in (a). Reaction R₃ lacks one substrate molecule of D in comparison to R₄, since N₄₄⁻−N₄₃⁻=1 and Nᵢ₄⁻−Nᵢ₃⁻=0 for every i≠4. Reactions R₅ and R₆ share the same components as substrates with same stoichiometry, and hence their fluxes are fully coupled under the assumption of mass action kinetic. The reactions R₆ and R₃ are fully coupled at steady state irrespective of the reaction rate law, and, by transitivity, also the reactions R₅ and R₃. (c) Component D exhibits structurally constrained concentration due to the full coupling of reactions R₅ and R₃. Component D also exhibits absolute concentration robustness, since its concentration depends only on fully coupled reactions.

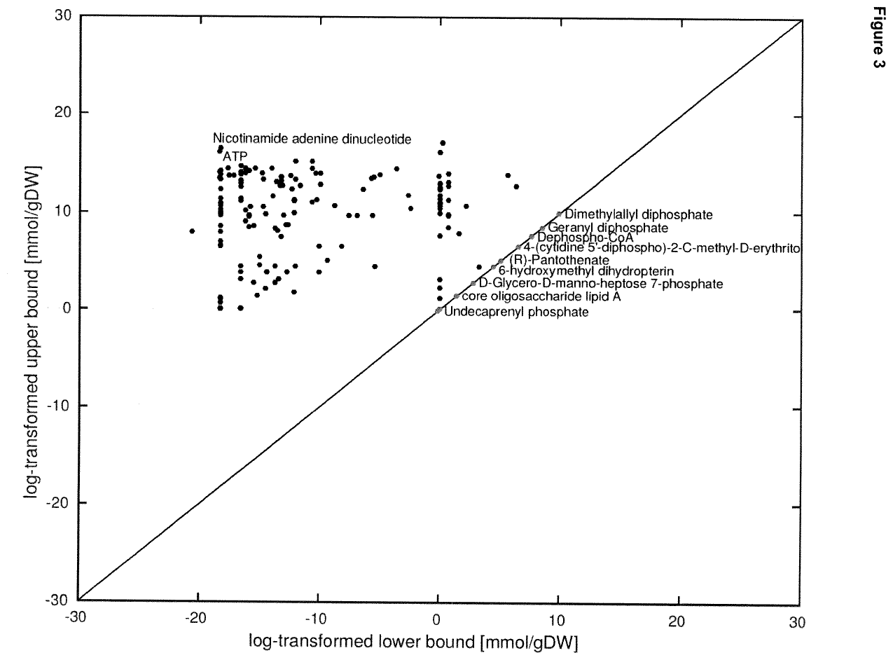

FIG. 2: Effect of missing information about rate constants on the accuracy of concentration range predictions for a large-scale kinetic model of E. coli. We consider 10-90% of the relevant rate constants to be unknown by random selection. We consider three scenarios for the substitution of missing ratios of rate constants: (i) equality (i.e., kinetic rate constants are assumed to be the same), (ii) the mean, or (iii) the median of the ratios of relevant rate constants that are still present in the model. Shown are the boxplots (red lines inside each box denote the corresponding medians) of the resulting Pearson correlation coefficients between the predicted and simulated ranges over the SCC components in the kinetic model of E. coli.

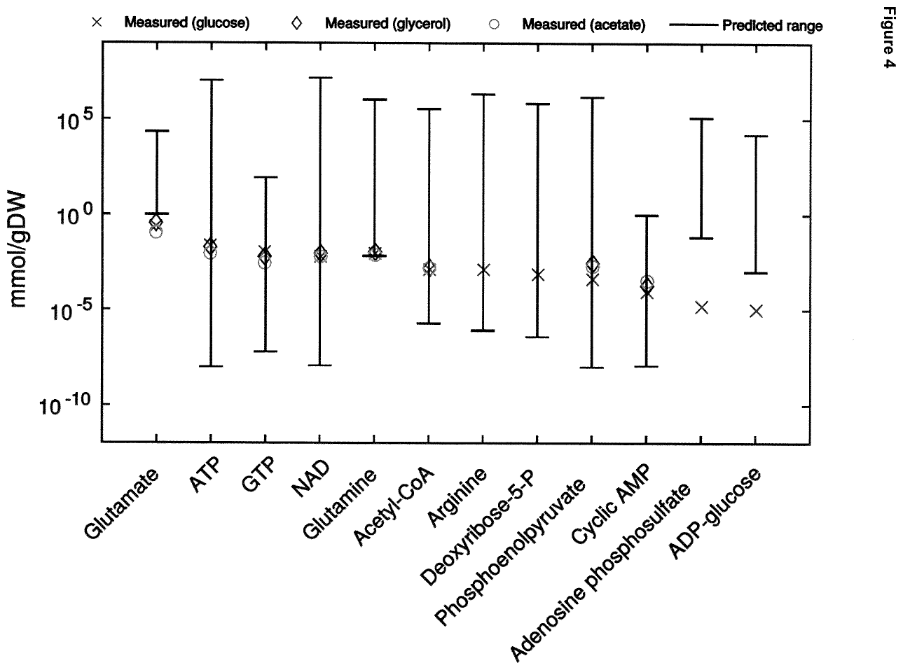

FIG. 3: Concentration bounds for cytosolic metabolites with structurally constrained concentrations in E. coli. The concentration bounds are determined following the proposed approach for 199 cytosolic metabolites by using bounds on flux ratios derived from integration of publicly available fluxomics data. Cytosolic metabolites with ratio of log-transformed upper to lower bound of at least 0.95 are highlighted in green. These metabolites are maintained in narrow ranges across the 17 investigated environmental scenarios. In contrast, NAD and ATP, marked in blue, vary considerably under the same scenarios. Missing ratios of kinetic rate constants are substituted by a ratio of one.

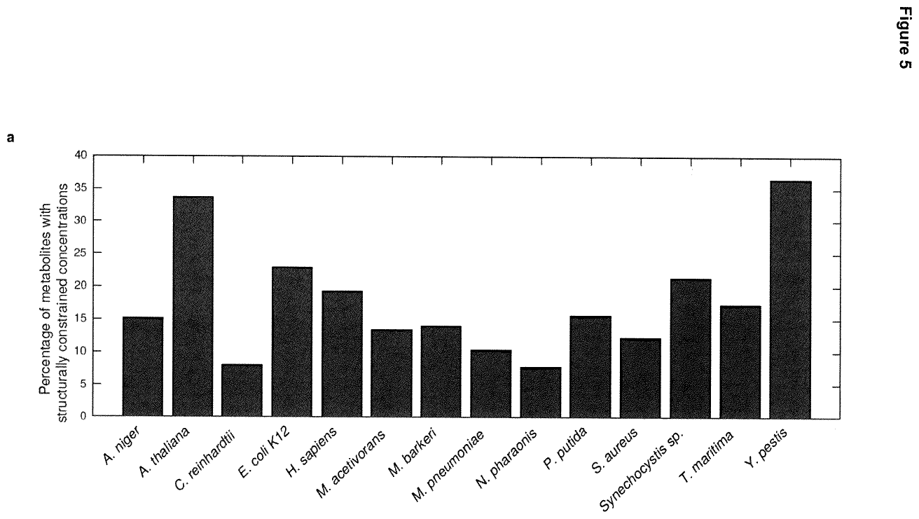

FIG. 4: Comparison of predicted ranges with measured metabolite concentrations. Comparison of the predicted concentration ranges of 12 intracellular metabolites in E. coli with absolute concentrations measured with acetate, glycerol, or glucose as carbon source using mass spectrometry. Altogether, 23 measurements fall within the predicted ranges, while those of Glutamate under the three carbon sources as well as Adenosine phosphosulfate and ADP-glucose with glucose as carbon source were measured below the predicted ranges.

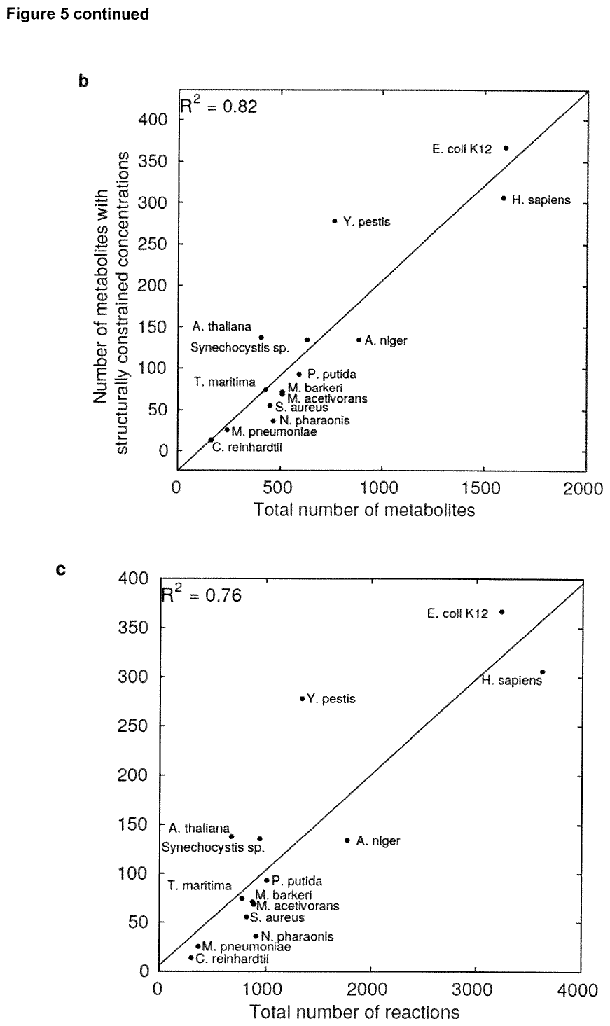

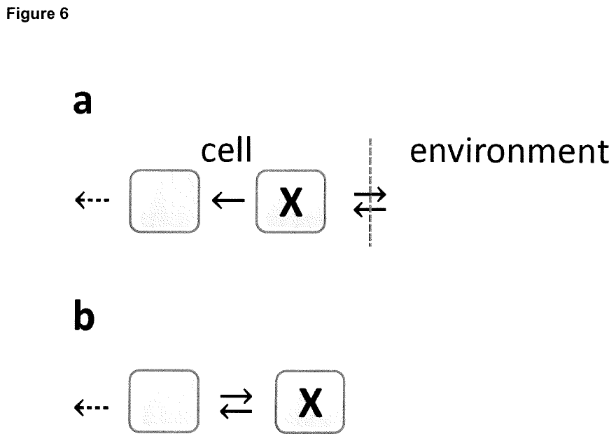

FIG. 5: Metabolites with structurally constrained concentration across species. (a) The fraction of metabolites with structurally constrained concentrations in 14 large-scale metabolic networks from all kingdoms of life. The number of these metabolites scales linearly with (b) the total number of metabolites (R²=0.82) and (c) the total number of reactions (R²=0.76).

FIG. 6: Network motifs for absolute concentration robustness. (a) Typical network motif for a component X that is exchanged with the environment, and (b) for a component X internal to the cell. Absolute concentration robustness is a direct consequence of the full coupling due to the steady-state assumption (in (b)) and to the full coupling due to shared substrate of same stoichiometry (in (a)).

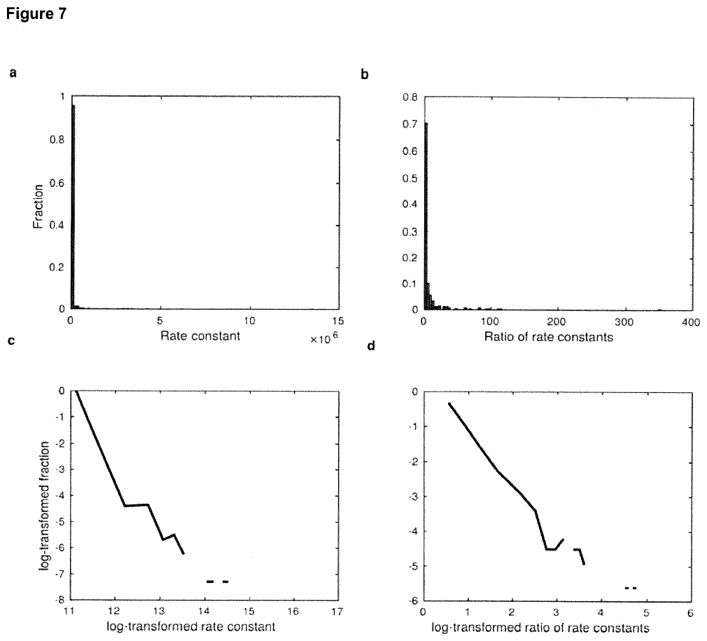

FIG. 7: Distribution of rate constants used in calculation of concentration ranges for SCC metabolites in a genome-sclae metabolic model of E. coli. Distribution of (a) the relevant rate constants and (b) their ratios for reactions coupled due to mass action kinetics; log-log distribution of (c) the relevant rate constants and (d) their ratios for reactions coupled due to mass action kinetics.

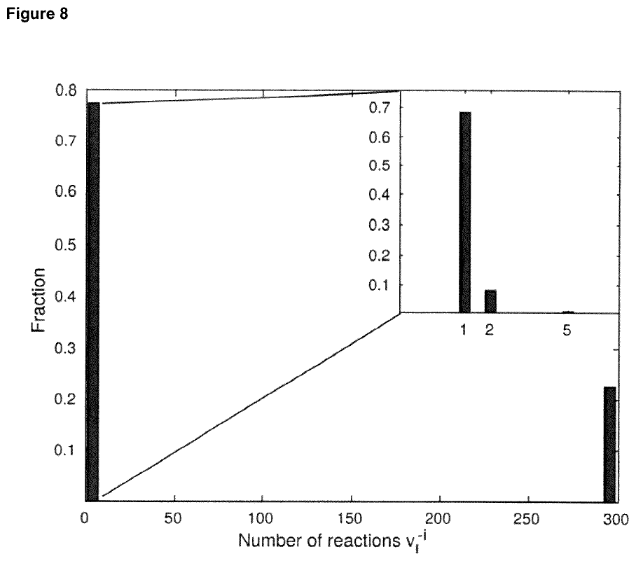

FIG. 8: Number of reactions in the set Rₗ⁻ⁱ lacking one molecule of Xᵢ in comparison to reaction Rₗ. For 68% of the reactions Rᵢ with relevant rate constants the set Q is unique. For about 25% of the reactions Rᵢ with relevant rate constants the set Rₗ⁻ⁱ.

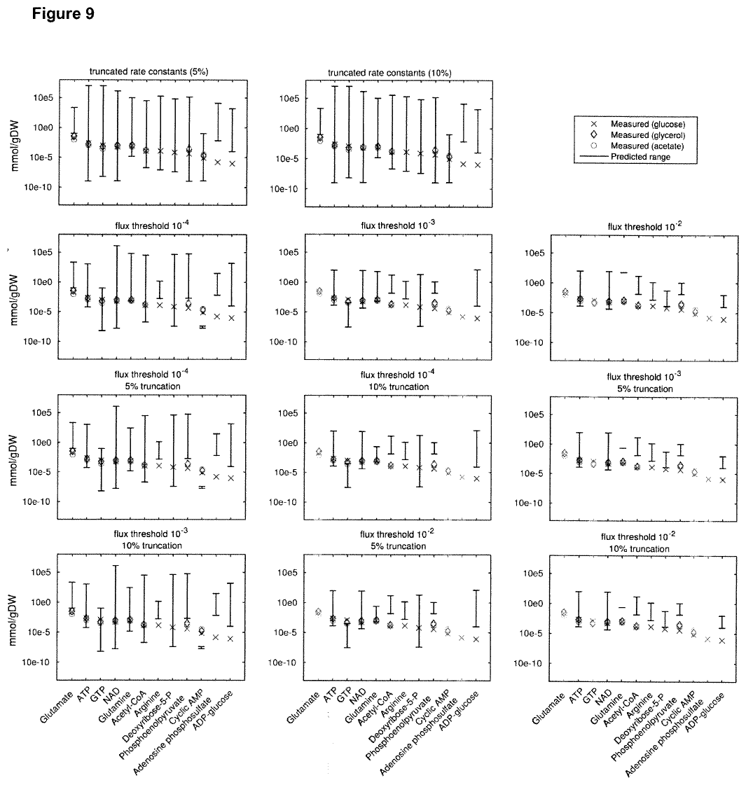

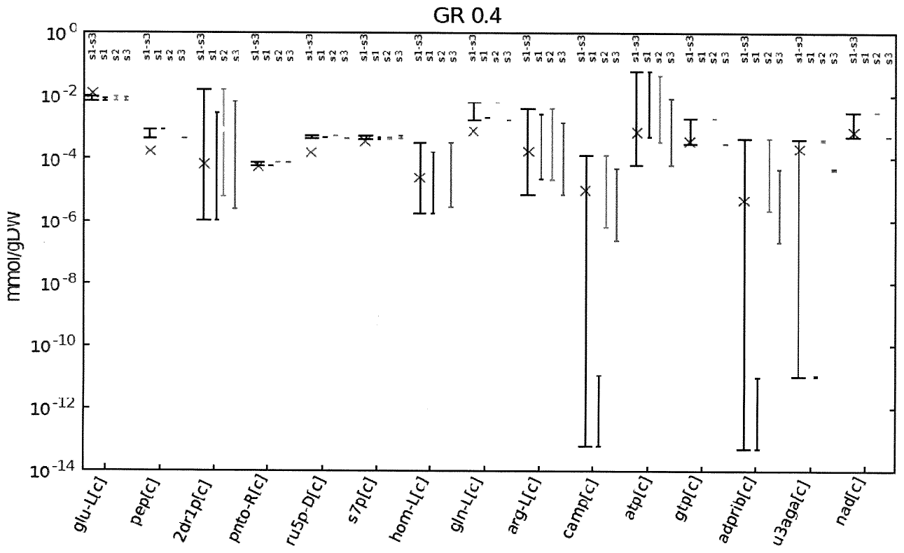

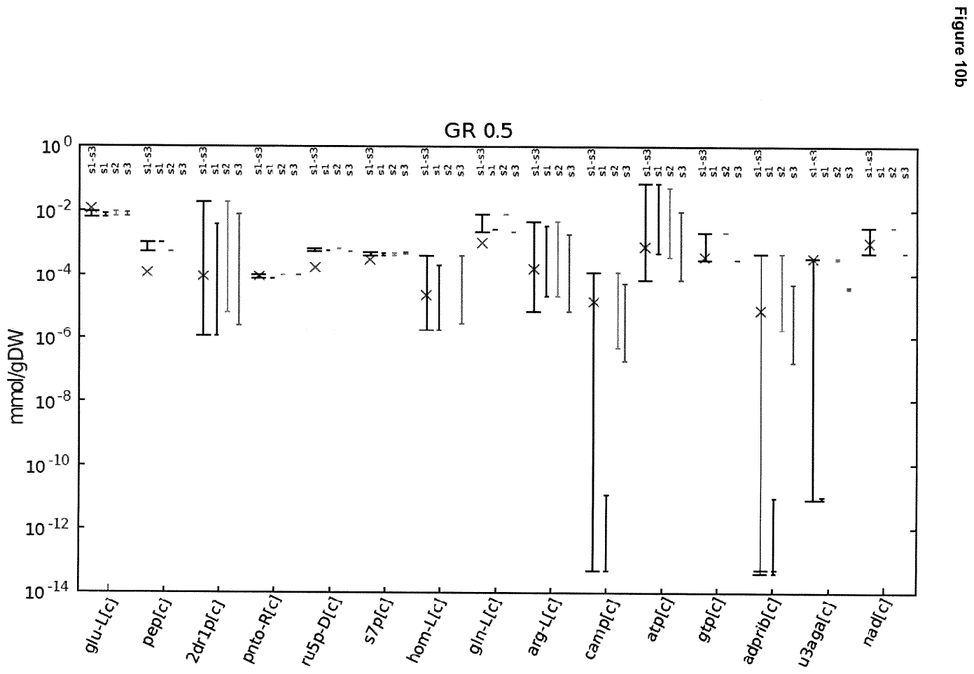

FIG. 9: Effect of relevant rate constants and reaction activity on the predicted ranges of 12 metabolites with experimentally determined concentrations. Concentration ranges were predicted using a truncated set of relevant constants, whereby the top/bottom 5 and 10% of the rate constants were considered unknown. In addition, only reactions with a flux value over a given threshold were used to calculate bounds on concentrations. Moreover, we inspect the combination of the previous additional constraints. For metabolites marked with a grey bar no concentration could be predicted under the additional constraints since all relevant rations come from excluded reactions.

FIG. 10: Comparison of predicted ranges with measured metabolite concentrations under the objective of optimizing ATP synthesis and sum of total flux. Comparison of the predicted concentration ranges for 15 intracellular metabolites in E. coli with absolute concentrations measured at growth rates (GR) of (a) 0.4, (b) 0.5 and (c) 0.7 h⁻¹. For metabolites with grey background, there is no access to measurements. The colored bars denote the predicted ranges from each of the three different replicates also indicated by s1, s2 and s3 at top of the figure, while the black bar represents the prediction over all replicates (s1-s3). The red cross denotes the measured value at the respective GR. For some metabolites there is no overlap between the colored bars, indicating poor reproducibility over the replicates in the reference scenario.

FIG. 11: Average measured and predicted concentration of SCC metabolites under different carbon sources. Each data point represents a SCC metabolite (different colors, see legend) under one carbon source (• fructose, ▪ galactose, ♦ glucose, [symbol] glycerol, [symbol] gluconate, ▴ pyruvate). The plotted predicted concentration value is the (max (c)−min (c))/2, where max (c) is the maximum predicted and min (c) the minimum predicted concentration. Note that due to numerical instabilities a concentration could not be calculated for all (SCC metabolite, carbon source) combinations; (a) concentration prediction using optimization of ATP synthesis and total flux (Spearman correlation 0.33) (b) concentration prediction using optimization of ATP synthesis (Spearman correlation 0.63).

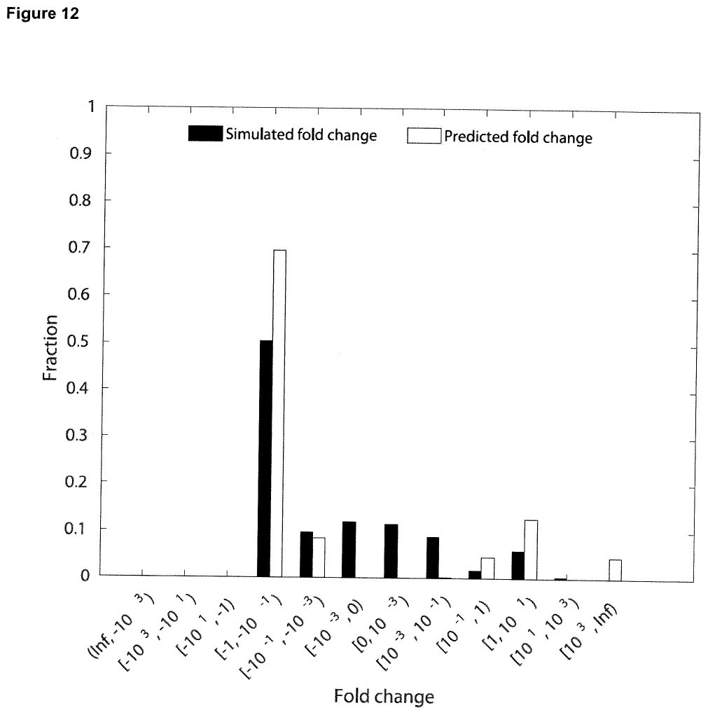

FIG. 12: Fold change in concentration of SCC metabolites upon reaction knock-out. The distribution of predicted and simulated fold change in concentration of 23 SCC metabolites over 929 single knock-out mutants for which a steady-state flux distribution could be simulated.

The examples illustrate the invention.

### Example 1: Methods

### Flux Coupling

Let (N)={v∈ [symbol] ⁿ|Nv=0, v≥0} be the steady-state flux cone for a given stoichiometric matrix N with n reactions, under the assumption that every reaction is irreversible. Here, we restrict our analysis to the subspace F⊂(N) by bounding the fluxes: F={v∈ [symbol] ⁿ|Nv=0, 0≤lb≤v≤ub}, where lb and ub are lower and upper flux bounds. We will refer to v∈F as the feasible flux distributions. A reaction Rᵢ is called blocked if for every v∈F, vᵢ=0. A pair of reactions Rᵢ and Rⱼ is called fully coupled, if for every v∈F, there exists λ>0, vᵢ=λvⱼ.

The minimum and maximum value for the ratio

$$\frac{{v}_{i}}{{v}_{j}}$$

over the flux distributions in F can be determined by the linear-fractional programming:

$$\begin{matrix}opt \frac{{v}_{i}}{{v}_{j}} \\ Nv = 0 \\ l b \leq v \leq ub ,\end{matrix}$$

which can be rewritten following the Charnes-Cooper transformation (Charnes, A. & Cooper, W. W. Programming with linear fractional functionals. Naval Research Logistics Quarterly 9, 181-186 (1962)) to the following linear program: opt vᵢ

$$\begin{matrix}Nv = 0 \\ {v}_{j} = 1 \\ t \cdot l b \leq v \leq t \cdot ub \\ t \geq 0.\end{matrix}$$

If the minimum and maximum values for the linear program are the same, then the reactions Rᵢ and Rⱼ are fully coupled. Such reactions can be efficiently computed for large-scale networks (Hackett, S. R. et al. Systems-level analysis of mechanisms regulating yeast metabolic flux. Science 354, doi: 10.1126/science.aaf2786 (2016); Burgard, A. P., Nikolaev, E. V., Schilling, C. H. & Maranas, C. D. Flux coupling analysis of genome-scale metabolic network reconstructions. Genome research 14, 301-312, doi: 10.1101/gr. 1926504 (2004)).

In addition, under the mass action kinetics, two reactions are fully coupled in any state of the system if they share the same substrates with the same stoichiometry. This leads to additional full couplings due to the transitivity of the relations, as demonstrated in the main text.

Components with Structurally Constrained Concentrations in Mass Action Networks

In the following, we present an algorithm determining SCC components under the assumption of mass action kinetics:

```
Input: network without blocked reactions and all reactions irreversible; list of fully coupled
    reactions (due to structure and mass action kinetic)
Output: components with structurally constrained concentration
for each component Xᵢ in network do
  Pᵢ ← all reactions with Xᵢ as a substrate
  Tᵢ ← all components consumed or produced by reactions in Pᵢ
  for each component Xⱼ in Tᵢ do
      Sⱼ ← set of all reactions in which Xⱼ appears as a substrate; Sⱼ⁻ⁱ ← ∅
        for each reaction Rₛ ϵ Sj
          if there is a reaction Rₛ⁻ⁱ that lacks one substrate molecule of Xᵢ in
          comparison to Rₛ
            add Rₛ⁻ⁱ to Sⱼ⁻ⁱ
          else
            Xᵢ has no SCC
          end if
        end for
        Pⱼ ← set of all reactions in which Xⱼ appears as a product;
        Pⱼ⁻ⁱ ← ∅
        for each reaction Rₚ ϵ Pⱼ
          if there is a reaction Rₚ⁻ⁱ that lacks one substrate molecule of Xᵢ in
          comparison to Rₚ
            add Rₚ⁻ⁱ to Pⱼ⁻ⁱ
          else
            Xᵢ has no SCC
          end if
        end for
        if all reactions in Pⱼ are mutually fully coupled and
          all reactions in Sⱼ⁻ⁱ are mutually fully coupled then
            Xᵢ has SCC
          end if
        else
          if all reactions in Sᵢ are mutually fully coupled and
            all reactions in Pⱼ⁻ⁱ are mutually fully coupled then
              Xᵢ has SCC
            end if
          end if
        end if
    end for
end for
```

### Effect of Missing Information on Rate Constants

To assess the effect of missing information about rate constants on the accuracy of the predicted concentration range, we simulated missing knowledge about parameters by randomly removing 10, 30, 50, 70 or 90% of the relevant kinetic rate constants. Such preferential removal is used to avoid bias due to removal of information in parts of the network that have no effect on the predictions of the concentration ranges. We compare the Pearson correlation coefficient between predicted and simulated concentration ranges for each percentage obtained over 100 random removals of rate constants.

Flux Fitting for the Model of E. coli

Let D={D₁, . . . , D_{d}} be a collection of flux profiles for a set of reactions Rⱼ^{α} obtained from fitting labeled metabolomics data to a model (Zamboni, N., Fendt, S. M., Ruhl, M. & Sauer, U. (13) C-based metabolic flux analysis. Nature protocols 4, 878-892, doi: 10.1038/nprot.2009.58 (2009)) and let N be a stoichiometric matrix of a network composed of m components that participate in n reactions. To obtain the minimum and maximum value for the flux ratio

$${r}_{p s} = \frac{{v}_{p}}{{v}_{s}}$$

used in the derivation above, we use the following linear programs:

### LP1:

```
min ε^{m} + ε^{p}
s.t. Nv = 0
  ∀Rⱼ ∉ Rⱼ^{α}
  ϵ ≤ lb ≤ vⱼ ≤ ub
  ∀Rⱼ ϵ Rⱼ^{α}
  vⱼ − εⱼ^{m} + εⱼ^{p} = Dⱼ
  ϵ ≤ lbⱼ^{α} ≤ vⱼ ≤ ubⱼ^{α}
  ε^{m}, ε^{p} ≥ 0,
```

where lb and ub are generic lower and upper bounds for the fluxes, ∈=10⁻⁵ is a parameter ensuring positivity of flux distributions, lb^{a} and ub^{a} are the lower and upper bounds for the measured fluxes, available from the respective confidence intervals, and ε^{m}, ε^{p} capturing the difference to the measurements (obtained by fitting a different, smaller model (Zamboni, N., Fendt, S. M., Ruhl, M. & Sauer, U. (13)C-based metabolic flux analysis. Nature protocols 4, 878-892, doi: 10.1038/nprot.2009.58 (2009)).

### LP2:

For every pair of reactions (k, l) which are coupled due to mass action kinetic, (i.e., share substrates of same stoichiometry), let the ratio rₖᵢ be available (see below). LP2 extends LP1 by including the additional constraint:

rₖᵢvᵢ=vₖ.

Ratios rₚₛ are then obtained over the flux distributions obtained by: (i) solving LP1 with each Dᵢ, from where the ratios rₖᵢ are determined for all pairs of reactions (k, l) that share substrate complexes, (ii) solving LP2 for each flux data set in D\Dᵢ with the ratios fixed according to the results of (i). In such a way, we determine the minimum and maximum values for the ratio rₚₛ while ensuring that fluxes that are coupled due to the kinetics are of fixed ratio in the considered fitting procedure.

Altogether we use 17 flux data sets from the E. coli strain MG1655 grown under glucose-limiting conditions with dilution rates varying between 0.04 and 0.41 h⁻¹ (Nanchen, A., Schicker, A. & Sauer, U. Nonlinear dependency of intracellular fluxes on growth rate in miniaturized continuous cultures of Escherichia coli. Applied and environmental microbiology 72, 1164-1172, doi: 10.1128/AEM.72.2.1164-1172.2006 (2006)).

### Cancer Models

To guarantee a positive steady state and to avoid presence of components participating in blocked reactions only, we unblocked the system by the introduction of import and export reactions around components being produced or consumed solely by blocked reactions. Such reactions are not included if they are already present in the original model.

### Example 2

Components with Structurally Constrained Concentrations

A network can be represented by the stoichiometric matrix, N=N⁺−N⁻, where N⁺ includes the stoichiometry of the products and N⁻ comprises the stoichiometry of the substrates of each reaction. In the following, we derive the conditions for structurally constrained robustness of component Xᵢ based on the ordinary differential equation (ODE) for the component Xⱼ (not necessarily different from Xᵢ) under the assumption that the reaction rates, (t), satisfy mass action kinetics, whereby

$${v}_{i} ( t ) = {θ}_{i} {\prod }_{j} {x}_{j}^{{N}_{ji}^{-}} ( t ) .$$

Let the ODE be specified by

$$\frac{{dx}_{j} ( t )}{dt} = {\sum }_{k ∈ {P}_{j}} {N}_{jk}^{+} {v}_{k} ( t ) - {\sum }_{l ∈ {S}_{j}} {N}_{jl}^{-} {v}_{l} ( t ) ,$$

where Pⱼ is the set of reactions with Xⱼ as one of their products and Sj is the set of reactions which have component Xⱼ as one of their substrates.

We consider the following two cases: (i) the concentration of Xᵢ appears in every vₖ(t) for which Nⱼₖ_{⁺}≠0 and for every vₖ(t) there exist a set of reactions Rₖ⁻ⁱ∈Pⱼ⁻ⁱ such that

$${v}_{k} ( t ) = {x}_{i} ( t ) \frac{{θ}_{k}}{{θ}_{k}^{- i}} {v}_{k}^{- i} ( t )$$

and (ii) the concentration of Xᵢ appears in every vᵢ(t) for which Nⱼₜ⁻≠0 and for every vₗ(t) there exist a set of reactions Rₗ⁻ⁱ∈Sⱼ⁻ⁱ such that

$${v}_{l} ( t ) = {x}_{i} ( t ) \frac{{θ}_{l}}{{θ}_{l}^{- i}} {v}_{l}^{- i} ( t ) .$$

### Case I:

The rates of a reaction Rk and a reaction from the set Rₖ⁻ⁱ are given by

$${v}_{k} ( t ) = {θ}_{k} {\prod }_{j} {x}_{j}^{{N}_{jk}^{-}} ( t ) = {θ}_{k} {\prod }_{j \neq i} {x}_{j}^{{N}_{jk}^{-}} ( t ) {x}_{i}^{{N}_{ik}^{-}} ( t ) = {θ}_{k} {x}_{i} ( t ) {\prod }_{j \neq i} {x}_{j}^{{N}_{jk}^{-}} ( t ) {x}_{i}^{{N}_{ik}^{-} - 1} ( t )$$

$$and$$

$${v}_{k}^{- i} ( t ) = {θ}_{k}^{- i} {\prod }_{j} {x}_{j}^{{N}_{{j}_{k}^{- 1}}^{-}} ( t ) = {θ}_{k}^{- i} {\prod }_{j \neq i} {x}_{j}^{{N}_{{j}_{k}^{- i}}^{-}} ( t ) {x}_{i}^{{N}_{{j}_{k}^{- i}}^{-}} ( t ) .$$

From rewriting the equation of vₖ⁻ⁱ(t) above we have that

$${\prod }_{j \neq i} {x}_{j}^{{N}_{{j}_{k}^{- i}}^{-}} ( t ) = \frac{{v}_{k}^{- i} ( t )}{{θ}_{k}^{- i} {x}_{i}^{{N}_{{j}_{k}^{- i}}^{-}} ( t )} .$$

### Since

$${N}_{jk}^{-} - {N}_{{j}_{k}^{- i}}^{-} = 0 = 0$$

for every j≠i and

$${N}_{ik}^{-} - {N}_{{j}_{k}^{- i}}^{-} = 1$$

we can rewrite the equation of (t) such that

$${v}_{k} ( t ) = \frac{{θ}_{k}}{{θ}_{k}^{- i}} {x}_{i} ( t ) {v}_{k}^{- i} ( t ) {{x}_{i}}^{{N}_{ik}^{-} - {N}_{{j}_{k}^{- i}}^{-} - 1} ( t ) = \frac{{θ}_{k}}{{θ}_{k}^{- i}} {x}_{i} ( t ) {v}_{k}^{- i} ( t ) .$$

The ODE for component Xⱼ revealing structurally constrained robustness of component Xᵢ is then given by:

$$\frac{{dx}_{j} ( t )}{dt} = {\sum }_{k ∈ {P}_{j}} {N}_{jk}^{+} {v}_{k} ( t ) - {\sum }_{l ∈ {S}_{j}} {N}_{jl}^{-} {v}_{l} ( t ) = {x}_{i} ( t ) {\sum }_{k ∈ {P}_{j}} {N}_{jk}^{+} \frac{{θ}_{k}}{{θ}_{k}^{- i}} {v}_{k}^{- i} ( t ) - {\sum }_{l ∈ {S}_{j}} {N}_{jl}^{-} {v}_{l} ( t ) .$$

Let p and s bet two reaction indices such that Nⱼₚ⁺≠0 and Nⱼₛ⁻≠0. In any positive state (t), we have that

$$\frac{{dx}_{j} ( t )}{dt} = {v}_{p}^{- i} ( t ) {x}_{i} ( t ) {\sum }_{k ∈ {P}_{j}} {N}_{jk}^{+} \frac{{θ}_{k}}{{θ}_{k}^{- i}} \frac{{v}_{k}^{- i} ( t )}{{v}_{p}^{- i} ( t )} - {v}_{s} ( t ) {\sum }_{l ∈ {S}_{j}} {N}_{jl}^{-} \frac{{v}_{l} ( t )}{{v}_{s} ( t )} .$$

In a steady state then

$${v}_{p}^{- i} {x}_{i} {\sum }_{k ∈ {P}_{j}} {N}_{jk}^{+} \frac{{θ}_{k}}{{θ}_{k}^{- i}} \frac{{v}_{k}^{- i}}{{v}_{p}^{- i}} - {v}_{s} {\sum }_{l ∈ {S}_{j}} {N}_{jl}^{-} \frac{{v}_{l}}{{v}_{s}} = 0.$$

It for every

$${N}_{{jp}^{+}} \neq 0 , \frac{{v}_{k}^{- i}}{{v}_{p}^{- i}}$$

is constant because either reactions Rₖ⁻ⁱ and Rₚ⁻ⁱ are fully coupled or share the same substrates, then

$${\sum }_{k ∈ {P}_{j}} {N}_{jk}^{+} \frac{{θ}_{k}}{{θ}_{k}^{- i}} \frac{{v}_{k}^{- i}}{{v}_{p}^{- i}} = {σ}_{p}^{- i}$$

is a constant that only depends on a subset of rate constants and the network structure. Moreover, if for every

$${N}_{{jl}^{-}} \neq 0 , \frac{{v}_{l}}{{v}_{s}}$$

is constant because either reactions Rₗ and Rₛ are fully coupled or share the same substrates, then

$${\sum }_{l ∈ {S}_{j}} {N}_{{jl}^{-}} \frac{{v}_{l}}{{v}_{s}} = {σ}_{s}$$

is a constant, too, which in the simplest case when all reactions in Sⱼ are fully coupled irrespective of the kinetic rate law, only depends on the network structure.

Therefore,

$${v}_{p}^{{-}_{i}} {x}_{i} {σ}_{p}^{{-}_{i}} - {v}_{s} {σ}_{s} = 0 , and {x}_{i} = \frac{{σ}_{s}}{{σ}_{p}^{- i}} \frac{{v}_{s}}{{v}_{p}^{- i}} .$$

Let Q be a subset of Pⱼ⁻ⁱ such that for every reaction Rₖ∈Pⱼ there is one reaction Rₖ⁻ⁱ∈Q. Since the reaction indices p and s are arbitrarily chosen, the concentration range of component Xᵢ for a given subset Q over a given set of flux distributions, F, is given as

$${min}_{\{ Q , F , {S}_{j} \}} \frac{{v}_{s}}{{v}_{p}^{- i}} \frac{{σ}_{s}}{{σ}_{p}^{- i}} \leq {x}_{i} \leq {max}_{\{ Q , F , {S}_{j} \}} \frac{{v}_{s}}{{v}_{p}^{- i}} \frac{{σ}_{s}}{{σ}_{p}^{- i}} .$$

### Case II:

The rates of a reaction Rₗ and a reaction from the set Rᵢ⁻ⁱ are given by

$${v}_{l} ( t ) = {θ}_{l} {\prod }_{j} {x}_{j}^{{N}_{jl}^{-}} ( t ) = {θ}_{i} {\prod }_{j \neq i} {x}_{j}^{{N}_{jl}^{-}} ( t ) {x}_{i}^{{N}_{il}^{-}} ( t ) = {θ}_{l} {x}_{i} ( t ) {\prod }_{j \neq i} {x}_{j}^{{N}_{jl}^{-}} ( t ) {x}_{i}^{{N}_{il}^{-} - 1} ( t ) \\ and \\ {v}_{l}^{- i} ( t ) = {θ}_{l}^{- i} {\prod }_{j} {x}_{j}^{{N}_{{j}_{i}^{- i}}^{-}} ( t ) = {θ}_{l}^{- i} {\prod }_{j \neq i} {x}_{j}^{{N}_{{j}_{l}^{- i}}^{-}} ( t ) {x}_{i}^{{N}_{{j}_{l}^{- i}}^{-}} ( t ) .$$

From rewriting the equation of vₗ⁻ⁱ(t) above we have that

$${\prod }_{j \neq i} {x}_{j}^{{N}_{{j}_{l}^{- i}}^{-}} ( t ) = \frac{{v}_{l}^{- i} ( t )}{{θ}_{l}^{- i} {x}_{i}^{{N}_{{j}_{l}^{- i}}^{-}} ( t )} .$$

### Since

$${N}_{jl}^{-} - {N}_{{j}_{l}^{- i}}^{-} = 0$$

for every j≠i and

$${N}_{il}^{-} - {N}_{{j}_{l}^{- i}}^{-} = 1$$

we call rewrite the equation of (t) such that

$${v}_{l} ( t ) = \frac{{θ}_{l}}{{θ}_{l}^{- i}} {x}_{i} ( t ) {v}_{l}^{- i} ( t ) {x}_{i}^{{N}_{il}^{-} - {N}_{{j}_{l}^{- i}}^{-} - 1} ( t ) = \frac{{θ}_{l}}{{θ}_{l}^{- i}} {x}_{i} ( t ) {v}_{l}^{- i} ( t ) .$$

The ODE for component Xⱼ revealing structurally constrained robustness of component Xᵢ is then given by:

$$\frac{{dx}_{j} ( t )}{dt} = {\sum }_{k ∈ {P}_{j}} {N}_{jk}^{+} {v}_{k} ( t ) - {\sum }_{l ∈ {S}_{j}} {N}_{jl}^{-} {v}_{l} ( t ) = {\sum }_{k ∈ {P}_{j}} {N}_{jk}^{+} {v}_{k} ( t ) - {x}_{i} ( t ) {\sum }_{l ∈ {S}_{j}} {N}_{jl}^{-} \frac{{θ}_{l}}{{θ}_{l}^{- i}} {v}_{l}^{- i} ( t ) .$$

Let p and s bet two reaction indices such that Nⱼₚ⁺≠0 and Nⱼₛ⁻≠0. In any positive state (t), we have that

$$\frac{{dx}_{j} ( t )}{dt} = {v}_{p} ( t ) {\sum }_{k ∈ {P}_{j}} {N}_{jk}^{+} \frac{{v}_{k} ( t )}{{v}_{p} ( t )} - {v}_{s}^{- i} ( t ) {x}_{i} ( t ) {\sum }_{l ∈ {S}_{j}} {N}_{jl}^{-} \frac{{θ}_{l}}{{θ}_{l}^{- i}} \frac{{v}_{l}^{- i} ( t )}{{v}_{s}^{- i} ( t )} .$$

In a steady state then

$${v}_{p} {\sum }_{k ∈ {P}_{j}} {N}_{jk}^{+} \frac{{v}_{k}}{{v}_{p}} - {v}_{s}^{- i} {x}_{i} {\sum }_{i ∈ {S}_{j}} {N}_{jl}^{-} \frac{{θ}_{l}}{{θ}_{l}^{- i}} \frac{{v}_{l}^{- i}}{{v}_{s}^{- i}} = 0.$$

If for every

$${N}_{jp}^{+} \neq 0 , \frac{{v}_{k}}{{v}_{p}}$$

is constant because either reactions Rₖ and Rₚ are fully coupled or share the same substrates, then

$${\sum }_{k ∈ {p}_{j}} {N}_{jk}^{+} \frac{{v}_{k}}{{v}_{p}} = {σ}_{p}$$

is a constant that, in the simplest case when all reactions in Pⱼ are fully coupled irrespective of the kinetic rate law, depends only on the network structure. Moreover, if for every

$${N}_{jl}^{-} \neq 0 , \frac{{v}_{l}^{- i}}{{v}_{s}^{- i}}$$

is constant because either reactions Rₗ⁻ⁱ and Rₛ⁻ⁱ are fully coupled or share the same substrates, then

$${\sum }_{l ∈ {S}_{j}} {N}_{jl}^{-} \frac{{θ}_{l}}{{θ}_{l}^{- i}} \frac{{v}_{l}^{- i}}{{v}_{s}^{- i}} = {σ}_{s}^{- i} .$$

The constant σₛ⁻ⁱ then only depends on a subset of rate constants and the network structure.

Therefore,

$${v}_{p} {σ}_{p} - {v}_{s}^{- i} {x}_{i} {σ}_{s}^{- i} = 0 , and$$

$${x}_{i} = \frac{{σ}_{p}}{{σ}_{s}^{- i}} \frac{{v}_{p}}{{v}_{s}^{- i}} .$$

Let Q be a subset of Sⱼ⁻ⁱ such that for every reaction Rₗ∈Si there is one reaction Rₗ⁻ⁱ∈Q. Since the reaction indices p and s are arbitrarily chosen, the concentration range of component Xᵢ for a given subset Q over a given set of flux distributions, F, is given as

$${min}_{\{ Q , F , {P}_{j} \}} \frac{{σ}_{p}}{{σ}_{s}^{- i}} \frac{{v}_{p}}{{v}_{s}^{- i}} \leq {x}_{i} \leq {max}_{\{ Q , F , {P}_{j} \}} \frac{{σ}_{p}}{{σ}_{s}^{- i}} \frac{{v}_{p}}{{v}_{s}^{- i}} .$$

As a result, the ranges for steady-state concentration x; can be expressed as a function of a set of given flux distributions, ratios of specific fluxes and constants that depend only on the structure of the network and values for a subset of rate constants. Since fluxes are the integrated outcome of transcription, translation, and post-translational modifications and their interplay with the environment and nutrient availability, our derivation provides a direct relation between concentration ranges, flux ratios, and rate constants.

### Example 3

Validation of the Approach with a Large-Scale Kinetic Model of E. coli

The proposed approach can be used to determine metabolite concentration ranges by using information about full coupling of reactions, selected ratios of reaction fluxes, and a limited set of reaction rate constants (i.e., the relevant reaction rate constants). To validate the predictions, we employ a detailed kinetic model of elementary metabolic reactions of E. coli (Khodayari, A., Zomorrodi, A. R., Liao, J. C. & Maranas, C. D. A kinetic model of Escherichia coli core metabolism satisfying multiple sets of mutant flux data. Metabolic engineering 25, 50-62, doi: 10.1016/j.ymben.2014.05.014 (2014)) from which these inputs are readily available. Of the 830 metabolites interconverted by 1,474 elementary reactions in the model, our approach determines that 23 of the 830 metabolites exhibit SCC. We use the kinetic model to simulate 100 steady states from different initial conditions.

We employ the Pearson correlation to assess if the predicted and simulated bounds agree across the metabolites with SCC. We determine that there is a perfect match between the predicted and simulated lower (1, p-value <10⁻⁶) and upper bounds (0.96, p-value <10⁻⁶) of the SCC metabolites, thus, demonstrating the validity of the theoretical derivation, given above (Table 1).

TABLE 1
Correlation between predicted concentration range and shadow price for 23
structurally constrained metabolites to the corresponding metabolic concentrations
obtained from 100 simulations of a kinetic model of E. coli core metabolism:

| Data | Correlation | p-value | Method |
|---|---|---|---|
| Predicted to simulated lower bound | 1 | <10⁻⁶ | Pearson |
| Predicted to simulated upper bound | 0.96 | <10⁻⁶ | Pearson |
| Range predicted to simulated | 0.96 | <10⁻⁶ | Pearson |
| Range predicted to simulated | 0.93 | <10⁻⁶ | Spearman |
| Log-transformed range predicted to simulated | 0.98 | <10⁻⁶ | Pearson |
| Shadow price to simulated range | −0.2 | 0.37 | Pearson |
| Shadow price to simulated range | −0.06 | 0.78 | Spearman |
| Shadow price to simulated log-transformed range | −0.06 | 0.77 | Pearson |
| Shadow price to CV over simulated concentrations | −0.2 | 0.35 | Pearson |
| Shadow price to log-transformed CV over simulated | −0.18 | 0.42 | Pearson |
| concentrations | | | |

It has been recently proposed that the shadow prices of metabolites can be used to quantify the ranges of metabolite concentrations, under the assumption that the cellular system optimizes an objective (Reznik, E., Mehta, P. & Segre, D. Flux imbalance analysis and the sensitivity of cellular growth to changes in metabolite pools. PLOS computational biology 9, e1003195, doi: 10.1371/journal.pcbi. 1003195 (2013)). To compare the performance of shadow prices as a measure of metabolite concentration ranges, we employ the stoichiometric matrix of the analyzed kinetic model by using the maximization of metabolic exchange fluxes as cellular objective, shown to outperform yield as a predictor of growth rate (Zarecki, R. et al. Maximal sum of metabolic exchange fluxes outperforms biomass yield as a predictor of growth rate of microorganisms. PloS one 9, e98372, doi: 10.1371/journal.pone.0098372 (2014)). We observe that for the analyzed model and the physiologically relevant objective, the calculated shadow prices cannot be used as indicators of concentration variability due to the weak negative correlation with the concentration ranges as well as with the coefficients of variation of the SCC metabolites (Table 1). These findings point out that our approach, in absence of a cellular objective but with knowledge about a few rate constants, outperforms the existing contender for quantifying concentration ranges in large-scale metabolic networks.

### Example 4

Effects of Missing Information about Rate Constants

While the full reaction couplings considered by our approach can be readily obtained given the structure of the network and flux ratios are increasingly available from labeling approaches (Horl, M., Schnidder, J., Sauer, U. & Zamboni, N. Non-stationary (13)C-metabolic flux ratio analysis. Biotechnology and bioengineering 110, 3164-3176, doi: 10.1002/bit.25004 (2013)), the resulting predictions can be affected by missing information about rate constants. To assess the effect of missing information on the accuracy of predictions, we consider the cases that 10-90% of rate constants used in the derivation of the ranges for the metabolites with SCC are known (see Example 1). We consider three scenarios whereby the missing ratios of rate constants, appearing in Eq. (1), are substituted by: (i) a value of one, (ii) the mean, or (iii) the median of the ratios of relevant rate constants that are present (i.e., known) in the model equation from which the conditions for SCC are established.

We find that the substitutions for the missing ratios of rate constants according to the three scenarios, as expected, decrease the Pearson correlation between predicted and simulated ranges over 100 instances of models in which relevant rate constants were removed at random (FIG. 2). Nevertheless, even when only 30% of the relevant rate constants are known for the cases (i) and (iii), we obtain a median Pearson correlation coefficient between the predicted and simulated ranges of at least 0.6 (FIG. 2). Substituting the missing ratio of rate constants with the mean of the ratios shows the largest variability over the 100 instances of models with partial knowledge of rate constants. The reason for this finding is that the distribution of rate constants and their ratios are highly left-skewed (FIG. 7). Therefore, we conclude that even in the absence of information about rate constants that matches the current state-of-the-art in E. coli, our approach provides qualitatively reliable estimates of concentration ranges in large-scale models.

### Example 5

Concentration Ranges in a Genome-Scale Metabolic Model of E. coli

Since rate constants are available for some of the enzymes in a genome-scale model of E. coli, we next investigate the effect of the flux ratios obtained from flux profiling experiments on the concentration ranges for metabolites with SCC. To this end, we next determine steady-state flux distributions closest to fluxes estimated from measurements ((Nanchen, A., Schicker, A. & Sauer, U. Nonlinear dependency of intracellular fluxes on growth rate in miniaturized continuous cultures of Escherichia coli. Applied and environmental microbiology 72, 1164-1172, doi: 10.1128/AEM.72.2.1164-1172.2006 (2006)), Example 1). We then employ Eq. (1) to predict the concentration ranges of 199 cytosolic metabolites (see Table 2), whereby missing ratios of rate constants are substituted either by a value of one or the median of the known ratios (cases (i) and (iii) above), which provide good qualitative agreement between predicted and simulated ranges. The subset of reactions Q appearing in Eq. (1) is unique for 68% of the metabolites with SCC (FIG. 8). For the remaining 32% of metabolites with SCC, we select one arbitrary Q and use it in the calculations.

TABLE 2
Concentration range for 199 cytosolic SCC metabolites in the E. coli model (see the
caption of FIG. 3 for details).

| | missing ratios set to median ratio | | |
|---|---|---|---|
| | lower | upper | |
| | bound | bound | range |
| Part 1: | [mmol/ | [mmol/ | [mmol/ |
| reaction name | gDW] | gDW] | gDW] |
| ‘1,4-dihydroxy-2-napthoyl-CoA’ | 1.60E−06 | 1.36E+02 | 1.36E+02 |
| ‘1,4-alpha-D-glucan’ | 1.96E−07 | 2.60E+05 | 2.60E+05 |
| ‘1,5-Diaminopentane’ | 9.13E−05 | 3.56E+06 | 3.56E+06 |
| ‘2,3-diaminopropionate’ | 3.59E−08 | 3.05E+00 | 3.05E+00 |
| ‘2-Dehydro-3-deoxy-D-arabino-heptonate 7-phosphate’ | 1.48E−03 | 4.87E+04 | 4.87E+04 |
| ‘2-dodecanoyl-sn-glycerol 3-phosphate’ | 3.59E−08 | 3.05E+00 | 3.05E+00 |
| ‘2-Dehydro-3-deoxy-D-galactonate’ | 3.05E+00 | 1.38E+06 | 1.38E+06 |
| ‘2-Deoxy-D-ribose 1-phosphate’ | 1.96E−07 | 3.99E+06 | 3.99E+06 |
| ‘2-Deoxy-D-ribose 5-phosphate’ | 1.17E−06 | 2.00E+06 | 2.00E+06 |
| ‘2-hexadec-9-enoyl-sn-glycerol 3-phosphate’ | 3.59E−08 | 5.52E+04 | 5.52E+04 |
| ‘2-hexadecanoyl-sn-glycerol 3-phosphate’ | 3.59E−08 | 3.05E+00 | 3.05E+00 |
| ‘2-Methylcitrate’ | 1.10E−06 | 3.64E+06 | 3.64E+06 |
| ‘2-octadec-11-enoyl-sn-glycerol 3-phosphate’ | 3.59E−08 | 8.79E+04 | 8.79E+04 |
| ‘2-octadecanoyl-sn-glycerol 3-phosphate’ | 3.59E−08 | 3.05E+00 | 3.05E+00 |
| ‘2-phospho-4-(cytidine 5″-diphospho)-2-C-methyl-D-erythritol’ | 1.30E−04 | 2.02E+03 | 2.02E+03 |
| ‘2-succinyl-5-enolpyruvyl-6-hydroxy-3-cyclohexene-1-carboxylate’ | 8.72E−06 | 1.36E+02 | 1.36E+02 |
| ‘2-Succinyl-6-hydroxy-2,4-cyclohexadiene-1-carboxylate’ | 8.72E−06 | 1.36E+02 | 1.36E+02 |
| ‘2-tetradec-7-enoyl-sn-glycerol 3-phosphate’ | 3.59E−08 | 3.05E+00 | 3.05E+00 |
| ‘2-tetradecanoyl-sn-glycerol 3-phosphate’ | 3.59E−08 | 3.05E+00 | 3.05E+00 |
| ‘3″,5″-Cyclic GMP’ | 3.59E−08 | 3.05E+00 | 3.05E+00 |
| ‘(R)-3-hydroxy-cis-dodec-5-enoyl-[acyl-carrier protein]’ | 3.91E−07 | 4.94E+04 | 4.94E+04 |
| ‘(R)-3-hydroxy-cis-myristol-7-eoyl-[acyl-carrier protein]’ | 3.91E−07 | 4.94E+04 | 4.94E+04 |
| ‘(R)-3-hydroxy-cis-palm-9-eoyl-[acyl-carrier protein]’ | 3.91E−07 | 4.36E+04 | 4.36E+04 |
| ‘(R)-3-hydroxy-cis-vacc-11-enoyl-[acyl-carrier protein]’ | 3.91E−07 | 1.48E+04 | 1.48E+04 |
| ‘3-Phosphohydroxypyruvate’ | 1.24E−05 | 2.87E+06 | 2.87E+06 |
| ‘4-Aminobutanal’ | 1.25E+00 | 2.60E+07 | 2.60E+07 |
| ‘4-amino-4-deoxychorismate’ | 1.74E−05 | 1.13E+07 | 1.13E+07 |
| ‘4-(cytidine 5″-diphospho)-2-C-methyl-D-erythritol’ | 2.02E+03 | 2.02E+03 | 1.15E−07 |
| ‘p-Cresol’ | 1.96E−07 | 6.49E+01 | 6.49E+01 |
| ‘4-Hydroxy-2-oxopentanoate’ | 5.87E−07 | 5.68E+06 | 5.68E+06 |
| ‘4-Methyl-2-oxopentanoate’ | 1.07E−09 | 3.09E+03 | 3.09E+03 |
| ‘5-Amino-4-oxopentanoate’ | 8.27E+02 | 2.95E+06 | 2.95E+06 |
| ‘5-Amino-6-(5″-phosphoribitylamino)uracil’ | 1.60E−06 | 1.36E+02 | 1.36E+02 |
| ‘5″-deoxyribose’ | 1.17E−06 | 1.10E+05 | 1.10E+05 |
| ‘5-Methylthioadenosine’ | 3.59E−08 | 2.05E+03 | 2.05E+03 |
| ‘6-hydroxymethyl dihydropterin’ | 2.72E+02 | 2.72E+02 | 3.45E−08 |
| ‘Aminoacetaldehyde’ | 1.96E−07 | 7.50E+06 | 7.50E+06 |
| ‘Acetyl-CoA’ | 1.94E−06 | 3.34E+05 | 3.34E+05 |
| ‘N-Acetyl-L-glutamate’ | 8.73E−02 | 3.56E+04 | 3.56E+04 |
| ‘N-Acetyl-D-mannosamine’ | 3.05E+00 | 8.72E+05 | 8.72E+05 |
| ‘N-Acetylneuraminate’ | 1.96E−07 | 8.72E+05 | 8.72E+05 |
| ‘ADPglucose’ | 3.02E−03 | 4.70E+04 | 4.70E+04 |
| ‘ADP-D-glycero-D-manno-heptose’ | 3.13E−06 | 4.87E+01 | 4.87E+01 |
| ‘ADPribose’ | 3.59E−08 | 6.09E+00 | 6.09E+00 |
| ‘S-Adenosyl-L-homocysteine’ | 4.88E−06 | 1.06E+06 | 1.06E+06 |
| ‘2-Amino-4-hydroxy-6-(erythro-1,2,3-trihydroxypropyl)dihydropteridine | 3.20E−06 | 1.34E+04 | 1.34E+04 |
| triphosphate’ | | | |
| ‘D-Allose’ | 3.05E+00 | 8.64E+05 | 8.64E+05 |
| ‘Allantoin’ | 7.18E−08 | 2.75E+06 | 2.75E+06 |
| ‘N-Acetyl-D-glucosamine(anhydrous)N-Acetylmuramyl-tetrapeptide’ | 3.59E−08 | 8.12E+00 | 8.12E+00 |
| ‘1,6-anhydrous-N-Acetylmuramyl-tetrapeptide’ | 3.59E−08 | 9.14E+00 | 9.14E+00 |
| ‘P1,P4-Bis(5″-adenosyl) tetraphosphate’ | 3.59E−08 | 3.05E+00 | 3.05E+00 |
| ‘Adenosine 5″-phosphosulfate’ | 6.68E−02 | 1.23E+05 | 1.23E+05 |
| ‘D-Arabinose 5-phosphate’ | 1.00E−04 | 2.56E+05 | 2.56E+05 |
| ‘aerobactin minus Fe3’ | 1.62E−05 | 2.08E+05 | 2.08E+05 |
| ‘L-Arginine’ | 2.55E−06 | 5.99E+06 | 5.99E+06 |
| ‘L-ascorbate-6-phosphate’ | 3.59E−08 | 1.63E+04 | 1.63E+04 |
| ‘ATP’ | 3.05E−08 | 3.11E+07 | 3.11E+07 |
| ‘Butanal’ | 6.80E−05 | 2.00E+05 | 2.00E+05 |
| ‘cAMP’ | 3.59E−08 | 3.05E+00 | 3.05E+00 |
| ‘cis-dodec-5-enoyl-[acyl-carrier protein] (n-C12:1)’ | 6.09E+00 | 4.94E+04 | 4.94E+04 |
| ‘cis-dec-3-enoyl-[acyl-carrier protein] (n-C10:1)’ | 6.09E+00 | 4.94E+04 | 4.94E+04 |
| ‘CDP-1,2-didodecanoylglycerol’ | 5.50E−06 | 5.80E+03 | 5.80E+03 |
| ‘CDP-1,2-dihexadec-9-enoylglycerol’ | 9.45E−03 | 2.22E+06 | 2.22E+06 |
| ‘CDP-1,2-dihexadecanoylglycerol’ | 1.21E−02 | 2.50E+06 | 2.50E+06 |
| ‘CDP-1,2-dioctadec-11-enoylglycerol’ | 4.88E−03 | 7.66E+05 | 7.66E+05 |
| ‘CDP-1,2-dioctadecanoylglycerol’ | 5.50E−06 | 1.80E+06 | 1.80E+06 |
| ‘CDP-1,2-ditetradec-7-enoylglycerol’ | 5.50E−06 | 2.39E+06 | 2.39E+06 |
| ‘CDP-1,2-ditetradecanoylglycerol’ | 5.50E−06 | 2.59E+06 | 2.59E+06 |
| ‘Cys-Gly’ | 3.59E−08 | 1.74E+05 | 1.74E+05 |
| ‘chorismate’ | 4.36E−06 | 6.79E+01 | 6.79E+01 |
| ‘diacetylchitobiose-6-phosphate’ | 3.59E−08 | 8.44E+03 | 8.44E+03 |
| ‘core oligosaccharide lipid A’ | 1.22E+01 | 1.22E+01 | 0.00E+00 |
| ‘coprogen unloaded (no Fe(III))’ | 1.62E−05 | 6.50E+04 | 6.50E+04 |
| ‘D-Cysteine’ | 3.59E−08 | 1.34E+05 | 1.34E+05 |
| ‘L-Cysteine’ | 1.42E−04 | 1.23E+06 | 1.23E+06 |
| ‘L-Cystathionine’ | 3.59E−08 | 4.76E+04 | 4.76E+04 |
| ‘5″-Deoxyadenosine’ | 9.45E−07 | 2.84E+02 | 2.84E+02 |
| ‘2,3-dihydroxicinnamic acid’ | 3.13E−08 | 2.08E+06 | 2.08E+06 |
| ‘Dihydroneopterin monophosphate’ | 3.20E−06 | 2.72E+02 | 2.72E+02 |
| ‘3-(2,3-Dihydroxyphenyl)propanoate’ | 6.25E−08 | 5.68E+06 | 5.68E+06 |
| ‘Dihydropteroate’ | 1.26E−02 | 2.72E+02 | 2.72E+02 |
| ‘4,5-dihydroxy-2,3-pentanedione’ | 2.66E−05 | 1.06E+06 | 1.06E+06 |
| ‘dIMP’ | 3.59E−08 | 9.32E+00 | 9.32E+00 |
| ‘dITP’ | 3.59E−08 | 9.32E+00 | 9.32E+00 |
| ‘Dimethylallyl diphosphate’ | 1.99E+04 | 1.99E+04 | 1.21E−05 |
| ‘Dephospho-CoA’ | 2.00E+03 | 2.00E+03 | 1.92E−07 |
| ‘dTDP-4-dehydro-6-deoxy-D-glucose’ | 3.42E−07 | 1.22E+01 | 1.22E+01 |
| ‘dTDPglucose’ | 1.56E−06 | 2.44E+01 | 2.44E+01 |
| ‘D-Erythrose 4-phosphate’ | 1.06E−02 | 4.87E+04 | 4.87E+04 |
| ‘D-erythro-1-(Imidazol-4-yl)glycerol 3-phosphate’ | 1.96E−07 | 3.15E+06 | 3.15E+06 |
| ‘Enterochelin’ | 2.71E−06 | 3.63E+05 | 3.63E+05 |
| ‘D-Fructose 1-phosphate’ | 3.05E+00 | 3.94E+05 | 3.94E+05 |
| ‘ferric 2,3-dihydroxybenzoylserine’ | 1.96E−07 | 1.46E+02 | 1.46E+02 |
| ‘Fe(III)hydoxamate, unloaded’ | 1.62E−05 | 2.45E+05 | 2.45E+05 |
| ‘Ferrichrome minus Fe(III)’ | 1.62E−05 | 1.83E+01 | 1.83E+01 |
| ‘ferroxamine minus Fe(3)’ | 1.62E−05 | 2.21E+05 | 2.21E+05 |
| ‘2-(Formamido)-N1-(5-phospho-D-ribosyl)acetamidine’ | 6.09E+00 | 1.10E+06 | 1.10E+06 |
| ‘Glycerophosphoserine’ | 3.59E−08 | 3.17E+06 | 3.17E+06 |
| ‘galactosyl-glucosyl-inner core oligosaccharide lipid A’ | 1.22E+01 | 1.22E+01 | 0.00E+00 |
| ‘D-Galactonate’ | 1.96E−07 | 1.38E+06 | 1.38E+06 |
| ‘GDP-D-mannose’ | 3.59E−08 | 3.05E+00 | 3.05E+00 |
| ‘Guanosine 3″-diphosphate 5″-triphosphate’ | 3.59E−08 | 3.05E+00 | 3.05E+00 |
| ‘glucosyl-galactosyl-glucosyl-inner core oligosaccharide lipid A’ | 1.22E+01 | 1.22E+01 | 0.00E+00 |
| ‘glucosyl-inner core oligosaccharide lipid A’ | 1.22E+01 | 1.22E+01 | 0.00E+00 |
| ‘L-Glutamine’ | 2.03E−02 | 3.15E+06 | 3.15E+06 |
| ‘L-Glutamate’ | 3.05E+00 | 6.54E+04 | 6.54E+04 |
| ‘Glycerol 2-phosphate’ | 3.59E−08 | 2.72E+05 | 2.72E+05 |
| ‘Glutaryl-[acyl-carrier protein] methyl ester’ | 3.05E+00 | 3.05E+00 | 1.02E−14 |
| ‘D-Glycero-D-manno-heptose 1,7-bisphosphate’ | 5.75E−07 | 4.87E+01 | 4.87E+01 |
| ‘D-Glycero-D-manno-heptose 7-phosphate’ | 4.87E+01 | 4.87E+01 | 0.00E+00 |
| ‘Geranyl diphosphate’ | 4.91E+03 | 4.91 E+03 | 2.98E−06 |
| ‘GTP’ | 1.96E−07 | 2.72E+02 | 2.72E+02 |
| ‘Glutathionylspermidine’ | 3.59E−08 | 3.05E+00 | 3.05E+00 |
| ‘H+ | 1.74E−05 | 1.92E+06 | 1.92E+06 |
| ‘H2O’ | 7.67E+01 | 2.72E+02 | 1.95E+02 |
| ‘cis-hexadec-9-enoyl-[acyl-carrier protein] (n-C16:1)’ | 6.09E+00 | 1.48E+04 | 1.48E+04 |
| ‘3-Hydroxyglutaryl-[acyl-carrier protein] methyl ester’ | 1.96E−07 | 3.05E+00 | 3.05E+00 |
| ‘heptosyl-heptosyl-kdo2-lipidA’ | 1.22E+01 | 1.22E+01 | 0.00E+00 |
| ‘L-Histidinol phosphate’ | 2.73E−07 | 2.80E+04 | 2.80E+04 |
| ‘2-Hydroxy-6-oxonona-2,4-diene-1,9-dioate’ | 7.18E−08 | 2.75E+06 | 2.75E+06 |
| ‘2-hydroxy-6-ketononatrienedioate’ | 3.59E−08 | 7.30E+05 | 7.30E+05 |
| ‘Hydroxymethylbilane’ | 1.33E−05 | 7.38E+05 | 7.38E+05 |
| ‘L-Homoserine’ | 4.82E−07 | 1.78E+04 | 1.78E+04 |
| ‘heptosyl-phospho-heptosyl-heptosyl-kdo2-lipidA’ | 1.22E+01 | 1.22E+01 | 0.00E+00 |
| ‘3-Hydroxypimeloyl-[acyl-carrier protein] methyl ester’ | 1.96E−07 | 3.05E+00 | 3.05E+00 |
| ‘Iminoaspartate’ | 8.88E−07 | 6.85E+02 | 6.85E+02 |
| ‘Isochorismate’ | 1.31E−04 | 1.36E+02 | 1.36E+02 |
| ‘inner core oligosaccharide lipid A (E. coli)’ | 1.22E+01 | 1.22E+01 | 0.00E+00 |
| ‘3-(Imidazol-4-yl)-2-oxopropyl phosphate’ | 3.60E−06 | 1.58E+06 | 1.58E+06 |
| ‘Isopentenyl diphosphate’ | 4.86E+00 | 2.76E+03 | 2.75E+03 |
| ‘KDO(2)-lipid IV(A)’ | 3.05E+00 | 1.28E+05 | 1.28E+05 |
| ‘3-Deoxy-D-manno-octulosonate 8-phosphate’ | 1.01E−05 | 1.97E+04 | 1.97E+04 |
| ‘(R)-S-Lactoylglutathione’ | 2.73E−07 | 5.58E+06 | 5.58E+06 |
| ‘cold adapted KDO(2)-lipid (A)’ | 3.05E+00 | 1.00E+05 | 1.00E+05 |
| ‘Lipid A Disaccharide’ | 2.74E+01 | 1.28E+05 | 1.28E+05 |
| ‘lipoyl-AMP’ | 1.96E−07 | 3.05E+00 | 3.05E+00 |
| ‘Lipoate’ | 3.05E+00 | 3.05E+00 | 0.00E+00 |
| ‘L-Lyxose’ | 1.96E−07 | 2.13E+05 | 2.13E+05 |
| ‘malonyl-CoA methyl ester’ | 3.05E+00 | 3.05E+00 | 1.02E−14 |
| ‘2(alpha-D-Mannosyl-6-phosphate)-D-glycerate’ | 3.59E−08 | 3.19E+03 | 3.19E+03 |
| (2R,4S)-2-methyl-2,4-dihydroxydihydrofuran-3-one’ | 4.88E−06 | 1.06E+06 | 1.06E+06 |
| ‘5,10-Methenyltetrahydrofolate’ | 2.73E−07 | 7.87E+04 | 7.87E+04 |
| ‘(2R,4S)-2-methyl-2,3,3,4-tetrahydroxytetrahydrofuran’ | 2.66E−05 | 1.06E+06 | 1.06E+06 |
| ‘Nicotinamide adenine dinucleotide’ | 3.59E−08 | 3.13E+07 | 3.13E+07 |
| ‘2-Oxopent-4-enoate’ | 1.08E−07 | 2.75E+06 | 2.75E+06 |
| ‘Oxamate’ | 2.81E−07 | 5.04E+06 | 5.04E+06 |
| ‘phosphatidylethanolamine (didodecanoyl, n-C12:0)’ | 6.09E+00 | 2.53E+05 | 2.53E+05 |
| ‘phosphatidylethanolamine (ditetradecanoyl, n-C14:0)’ | 6.09E+00 | 3.62E+06 | 3.62E+06 |
| ‘phosphatidylethanolamine (ditetradec-7-enoyl, n-C14:1)’ | 6.09E+00 | 1.59E+06 | 1.59E+06 |
| ‘phosphatidylethanolamine (dioctadecanoyl, n-C18:0)’ | 6.09E+00 | 3.60E+06 | 3.60E+06 |
| ‘Phosphoenolpyruvate’ | 3.05E−08 | 4.23E+06 | 4.23E+06 |
| ‘Phosphatidylglycerophosphate (didodecanoyl, n-C12:0)’ | 3.05E+00 | 1.89E+05 | 1.89E+05 |
| ‘Phosphatidylglycerophosphate (ditetradecanoyl, n-C14:0)’ | 3.05E+00 | 9.14E+00 | 6.09E+00 |
| ‘Phosphatidylglycerophosphate (ditetradec-7-enoyl, n-C14:1)’ | 3.05E+00 | 3.05E+01 | 2.74E+01 |
| ‘Phosphatidylglycerophosphate (dihexadecanoyl, n-C16:0)’ | 3.05E+00 | 5.90E+03 | 5.89E+03 |
| ‘Phosphatidylglycerophosphate (dihexadec-9-enoyl, n-C16:1)’ | 3.05E+00 | 1.33E+06 | 1.33E+06 |
| ‘Phosphatidylglycerophosphate (dioctadecanoyl, n-C18:0)’ | 3.05E+00 | 2.76E+05 | 2.76E+05 |
| ‘Phosphatidylglycerophosphate (dioctadec-11-enoyl, n-C18:1)’ | 3.05E+00 | 1.77E+05 | 1.77E+05 |
| ‘Protoheme’ | 3.05E+00 | 7.38E+05 | 7.38E+05 |
| ‘O-Phospho-L-homoserine’ | 3.59E−08 | 7.51E+04 | 7.51E+04 |
| ‘Pimeloyl-[acyl-carrier protein]’ | 2.66E−04 | 6.80E+02 | 6.80E+02 |
| ‘Pimeloyl-[acyl-carrier protein] methyl ester’ | 3.59E−08 | 3.05E+00 | 3.05E+00 |
| ‘(R)-Pantothenate’ | 1.59E+02 | 1.59E+02 | 1.53E−08 |
| ‘N-(5-Phospho-D-ribosyl)anthranilate’ | 5.48E−07 | 1.68E+04 | 1.68E+04 |
| ‘1-(5-Phosphoribosyl)-AMP’ | 2.73E−07 | 2.80E+04 | 2.80E+04 |
| ‘1-(5-Phosphoribosyl)-ATP’ | 2.73E−07 | 2.80E+04 | 2.80E+04 |
| ‘L-Prolinylglycine’ | 3.59E−08 | 6.54E+04 | 6.54E+04 |
| ‘5-Phospho-alpha-D-ribose 1-diphosphate’ | 9.28E−01 | 9.61E+05 | 9.61E+05 |
| ‘O-Phospho-L-serine’ | 3.82E−07 | 4.54E+06 | 4.54E+06 |
| ‘Putrescine’ | 1.42E−04 | 3.56E+06 | 3.56E+06 |
| ‘D-Ribose 1,5-bisphosphate’ | 3.05E+00 | 3.11E+07 | 3.11E+07 |
| ‘alpha-D-Ribose 1-phosphate’ | 3.05E+00 | 3.11E+07 | 3.11E+07 |
| ‘S-Ribosyl-L-homocysteine’ | 2.66E−05 | 1.06E+06 | 1.06E+06 |
| ‘D-Ribulose 5-phosphate’ | 1.74E−05 | 2.72E+02 | 2.72E+02 |
| ‘Sedoheptulose 7-phosphate’ | 3.13E−06 | 4.87E+01 | 4.87E+01 |
| ‘Sucrose 6-phosphate’ | 3.59E−08 | 2.46E+03 | 2.46E+03 |
| ‘Succinyl-CoA’ | 2.74E−07 | 3.64E+06 | 3.64E+06 |
| ‘N2-Succinyl-L-glutamate’ | 3.59E−08 | 4.47E+06 | 4.47E+06 |
| ‘N2-Succinyl-L-glutamate 5-semialdehyde’ | 6.80E−05 | 5.99E+06 | 5.99E+06 |
| ‘O-Succinyl-L-homoserine’ | 6.59E−06 | 4.76E+04 | 4.76E+04 |
| ‘D-tartrate’ | 1.96E−07 | 1.77E+06 | 1.77E+06 |
| ‘cis-tetradec-7-enoyl-[acyl-carrier protein] (n-C14:1)’ | 6.09E+00 | 5.73E+04 | 5.73E+04 |
| ‘Thiamin’ | 3.05E+00 | 6.49E+01 | 6.18E+01 |
| ‘Trehalose’ | 3.59E−08 | 1.64E+05 | 1.64E+05 |
| ‘alpha,alpha″-Trehalose 6-phosphate’ | 3.59E−08 | 1.64E+05 | 1.64E+05 |
| ‘tRNA (Glu)’ | 7.70E−02 | 5.90E+06 | 5.90E+06 |
| ‘UDP-2,3-bis(3-hydroxytetradecanoyl)glucosamine’ | 4.13E−06 | 9.86E+03 | 9.86E+03 |
| ‘UDP-3-O-(3-hydroxytetradecanoyl)-N-acetylglucosamine’ | 8.26E−06 | 1.97E+04 | 1.97E+04 |
| ‘UDP-3-O-(3-hydroxytetradecanoyl)-D-glucosamine’ | 4.49E−04 | 1.28E+05 | 1.28E+05 |
| ‘undecaprenyl phosphate-4-amino-4-formyl-L-arabinose’ | 3.59E−08 | 3.05E+00 | 3.05E+00 |
| ‘undecaprenyl phosphate-4-amino-4-deoxy-L-arabinose’ | 1.96E−07 | 3.05E+00 | 3.05E+00 |
| ‘Undecaprenyl-diphospho-N-acetylmuramoyl-(N-acetylglucosamine)-L- | 2.57E−04 | 4.68E+02 | 4.68E+02 |
| ‘ala-D-glu-meso-2,6-diaminopimeloyl-D-ala-D-ala’ | | | |
| ‘Undecaprenyl-diphospho-N-acetylmuramoyl-L-alanyl-D-glutamyl-meso- | 1.69E+03 | 1.07E+06 | 1.07E+06 |
| 2,6-diaminopimeloyl-D-alanyl-D-alanine’ | | | |
| ‘Undecaprenyl phosphate’ | 2.44E+00 | 2.44E+00 | 1.02E−14 |
| ‘UDP-N-acetylmuramoyl-L-alanyl-D-gamma-glutamyl-meso-2,6- | 3.93E−07 | 1.10E+05 | 1.10E+05 |
| diaminopimelate-D-alanine | | | |
| ‘Undecaprenyl-diphospho N-acetylglucosamine-N- | 7.82E−07 | 1.22E+01 | 1.22E+01 |
| acetylmannosaminuronate-N-acetamido-4,6-dideoxy-D-galactose’ | | | |
| ‘Ureidoacrylate peracid’ | 3.59E−08 | 1.81E+06 | 1.81E+06 |
| ‘Xanthine’ | 6.80E−05 | 1.22E+07 | 1.22E+07 |
| ‘XTP’ | 3.59E−08 | 6.09E+00 | 6.09E+00 |
| | missing ratios set to ratio of 1 | | |
| | lower | upper | |
| | bound | bound | range |
| Part 2: | [mmol/ | [mmol/ | [mmol/ |
| reaction name | gDW] | gDW] | gDW] |
| ‘1,4-dihydroxy-2-napthoyl-CoA’ | 5.26E−07 | 4.46E+01 | 4.46E+001 |
| ‘1,4-alpha-D-glucan’ | 6.42E−08 | 8.52E+04 | 8.52E+004 |
| ‘1,5-Diaminopentane’ | 3.00E−05 | 1.17E+06 | 1.17E+006 |
| ‘2,3-diaminopropionate’ | 1.18E−08 | 1.00E+00 | 1.00E+000 |
| ‘2-Dehydro-3-deoxy-D-arabino-heptonate 7-phosphate’ | 4.85E−04 | 1.60E+04 | 1.60E+004 |
| ‘2-dodecanoyl-sn-glycerol 3-phosphate’ | 1.18E−08 | 1.00E+00 | 1.00E+000 |
| ‘2-Dehydro-3-deoxy-D-galactonate’ | 1.00E+00 | 4.53E+05 | 4.53E+005 |
| ‘2-Deoxy-D-ribose 1-phosphate’ | 6.42E−08 | 1.31E+06 | 1.31E+006 |
| ‘2-Deoxy-D-ribose 5-phosphate’ | 3.85E−07 | 6.55E+05 | 6.55E+005 |
| ‘2-hexadec-9-enoyl-sn-glycerol 3-phosphate’ | 1.18E−08 | 1.81E+04 | 1.81E+004 |
| ‘2-hexadecanoyl-sn-glycerol 3-phosphate’ | 1.18E−08 | 1.00E+00 | 1.00E+000 |
| ‘2-Methylcitrate’ | 3.60E−07 | 1.19E+06 | 1.19E+006 |
| ‘2-octadec-11-enoyl-sn-glycerol 3-phosphate’ | 1.18E−08 | 2.89E+04 | 2.89E+004 |
| ‘2-octadecanoyl-sn-glycerol 3-phosphate’ | 1.18E−08 | 1.00E+00 | 1.00E+000 |
| ‘2-phospho-4-(cytidine 5″-diphospho)-2-C-methyl-D-erythritol’ | 4.26E−05 | 6.63E+02 | 6.63E+002 |
| ‘2-succinyl-5-enolpyruvyl-6-hydroxy-3-cyclohexene-1-carboxylate’ | 2.87E−06 | 4.46E+01 | 4.46E+001 |
| ‘2-Succinyl-6-hydroxy-2,4-cyclohexadiene-1-carboxylate’ | 2.87E−06 | 4.46E+01 | 4.46E+001 |
| ‘2-tetradec-7-enoyl-sn-glycerol 3-phosphate’ | 1.18E−08 | 1.00E+00 | 1.00E+000 |
| ‘2-tetradecanoyl-sn-glycerol 3-phosphate’ | 1.18E−08 | 1.00E+00 | 1.00E+000 |
| ‘3″,5″-Cyclic GMP’ | 1.18E−08 | 1.00E+00 | 1.00E+000 |
| ‘(R)-3-hydroxy-cis-dodec-5-enoyl-[acyl-carrier protein]’ | 1.28E−07 | 1.62E+04 | 1.62E+004 |
| ‘(R)-3-hydroxy-cis-myristol-7-eoyl-[acyl-carrier protein]’ | 1.28E−07 | 1.62E+04 | 1.62E+004 |
| ‘(R)-3-hydroxy-cis-palm-9-eoyl-[acyl-carrier protein]’ | 1.28E−07 | 1.43E+04 | 1.43E+004 |
| ‘(R)-3-hydroxy-cis-vacc-11-enoyl-[acyl-carrier protein]’ | 1.28E−07 | 4.87E+03 | 4.87E+003 |
| ‘3-Phosphohydroxypyruvate’ | 4.07E−06 | 9.44E+05 | 9.44E+005 |
| ‘4-Aminobutanal’ | 1.25E+00 | 2.60E+07 | 2.60E+007 |
| ‘4-amino-4-deoxychorismate’ | 5.73E−06 | 3.70E+06 | 3.70E+006 |
| ‘4-(cytidine 5″-diphospho)-2-C-methyl-D-erythritol’ | 6.63E+02 | 6.63E+02 | 3.77E−008 |
| ‘p-Cresol’ | 6.42E−08 | 2.13E+01 | 2.13E+001 |
| ‘4-Hydroxy-2-oxopentanoate’ | 1.93E−07 | 1.86E+06 | 1.86E+006 |
| ‘4-Methyl-2-oxopentanoate’ | 1.07E−09 | 3.09E+03 | 3.09E+003 |
| ‘5-Amino-4-oxopentanoate’ | 2.72E+02 | 9.69E+05 | 9.69E+005 |
| ‘5-Amino-6-(5″-phosphoribitylamino)uracil’ | 5.26E−07 | 4.46E+01 | 4.46E+001 |
| ‘5″-deoxyribose’ | 3.85E−07 | 3.61E+04 | 3.61E+004 |
| ‘5-Methylthioadenosine’ | 1.18E−08 | 6.74E+02 | 6.74E+002 |
| ‘6-hydroxymethyl dihydropterin’ | 8.92E+01 | 8.92E+01 | 1.13E−008 |
| ‘Aminoacetaldehyde’ | 6.42E−08 | 2.46E+06 | 2.46E+006 |
| ‘Acetyl-CoA’ | 1.94E−06 | 3.34E+05 | 3.34E+005 |
| ‘N-Acetyl-L-glutamate’ | 8.73E−02 | 3.56E+04 | 3.56E+004 |
| ‘N-Acetyl-D-mannosamine’ | 1.00E+00 | 2.86E+05 | 2.86E+005 |
| ‘N-Acetylneuraminate’ | 6.42E−08 | 2.86E+05 | 2.86E+005 |
| ‘ADPglucose’ | 9.91E−04 | 1.54E+04 | 1.54E+004 |
| ‘ADP-D-glycero-D-manno-heptose’ | 1.03E−06 | 1.60E+01 | 1.60E+001 |
| ‘ADPribose’ | 1.18E−08 | 2.00E+00 | 2.00E+000 |
| ‘S-Adenosyl-L-homocysteine’ | 1.60E−06 | 3.50E+05 | 3.50E+005 |
| ‘2-Amino-4-hydroxy-6-(erythro-1,2,3- | 1.05E−06 | 4.40E+03 | 4.40E+003 |
| trihydroxypropyl)dihydropteridine triphosphate’ | | | |
| ‘D-Allose’ | 1.00E+00 | 2.84E+05 | 2.84E+005 |
| ‘Allantoin’ | 2.36E−08 | 9.04E+05 | 9.04E+005 |
| ‘N-Acetyl-D-glucosamine(anhydrous)N-Acetylmuramyl-tetrapeptide’ | 1.18E−08 | 2.67E+00 | 2.67E+000 |
| ‘1,6-anhydrous-N-Acetylmuramyl-tetrapeptide’ | 1.18E−08 | 3.00E+00 | 3.00E+000 |
| ‘P1,P4-Bis(5″-adenosyl) tetraphosphate’ | 1.18E−08 | 1.00E+00 | 1.00E+000 |
| ‘Adenosine 5″-phosphosulfate’ | 6.68E−02 | 1.23E+05 | 1.23E+005 |
| ‘D-Arabinose 5-phosphate’ | 3.29E−05 | 8.40E+04 | 8.40E+004 |
| ‘aerobactin minus Fe3’ | 5.33E−06 | 6.83E+04 | 6.83E+004 |
| ‘L-Arginine’ | 8.37E−07 | 1.97E+06 | 1.97E+006 |
| ‘L-ascorbate-6-phosphate’ | 1.18E−08 | 5.35E+03 | 5.35E+003 |
| ‘ATP’ | 1.00E−08 | 1.02E+07 | 1.02E+007 |
| ‘Butanal’ | 2.23E−05 | 6.58E+04 | 6.58E+004 |
| ‘cAMP’ | 1.18E−08 | 1.00E+00 | 1.00E+000 |
| ‘cis-dodec-5-enoyl-[acyl-carrier protein] (n-C12:1)’ | 2.00E+00 | 1.62E+04 | 1.62E+004 |
| ‘cis-dec-3-enoyl-[acyl-carrier protein] (n-C10:1)’ | 2.00E+00 | 1.62E+04 | 1.62E+004 |
| ‘CDP-1,2-didodecanoylglycerol’ | 1.81E−06 | 1.91E+03 | 1.91E+003 |
| ‘CDP-1,2-dihexadec-9-enoylglycerol’ | 3.10E−03 | 7.29E+05 | 7.29E+005 |
| ‘CDP-1,2-dihexadecanoylglycerol’ | 3.99E−03 | 8.21E+05 | 8.21E+005 |
| ‘CDP-1,2-dioctadec-11-enoylglycerol’ | 1.60E−03 | 2.52E+05 | 2.52E+005 |
| ‘CDP-1,2-dioctadecanoylglycerol’ | 1.81E−06 | 5.91E+05 | 5.91E+005 |
| ‘CDP-1,2-ditetradec-7-enoylglycerol’ | 1.81E−06 | 7.85E+05 | 7.85E+005 |
| ‘CDP-1,2-ditetradecanoylglycerol’ | 1.81E−06 | 8.51E+05 | 8.51E+005 |
| ‘Cys-Gly’ | 1.18E−08 | 5.70E+04 | 5.70E+004 |
| ‘chorismate’ | 1.43E−06 | 2.23E+01 | 2.23E+001 |
| ‘diacetylchitobiose-6-phosphate’ | 1.18E−08 | 2.77E+03 | 2.77E+003 |
| ‘core oligosaccharide lipid A’ | 4.00E+00 | 4.00E+00 | 0.00E+000 |
| ‘coprogen unloaded (no Fe(III))’ | 5.33E−06 | 2.14E+04 | 2.14E+004 |
| ‘D-Cysteine’ | 1.18E−08 | 4.41E+04 | 4.41E+004 |
| ‘L-Cysteine’ | 4.66E−05 | 4.04E+05 | 4.04E+005 |
| ‘L-Cystathionine’ | 1.18E−08 | 1.56E+04 | 1.56E+004 |
| ‘5″-Deoxyadenosine’ | 3.10E−07 | 9.32E+01 | 9.32E+001 |
| ‘2,3-dihydroxicinnamic acid’ | 1.03E−08 | 6.85E+05 | 6.85E+005 |
| ‘Dihydroneopterin monophosphate’ | 1.05E−06 | 8.92E+01 | 8.92E+001 |
| ‘3-(2,3-Dihydroxyphenyl)propanoate’ | 2.05E−08 | 1.86E+06 | 1.86E+006 |
| ‘Dihydropteroate’ | 4.15E−03 | 8.92E+01 | 8.92E+001 |
| ‘4,5-dihydroxy-2,3-pentanedione’ | 8.72E−06 | 3.50E+05 | 3.50E+005 |
| ‘dIMP’ | 1.18E−08 | 3.06E+00 | 3.06E+000 |
| ‘dITP’ | 1.18E−08 | 3.06E+00 | 3.06E+000 |
| ‘Dimethylallyl diphosphate’ | 1.99E+04 | 1.99E+04 | 1.21E−005 |
| ‘Dephospho-CoA’ | 2.00E+03 | 2.00E+03 | 1.92E−007 |
| ‘dTDP-4-dehydro-6-deoxy-D-glucose’ | 2.57E−07 | 4.00E+00 | 4.00E+000 |
| ‘dTDPglucose’ | 5.14E−07 | 8.00E+00 | 8.00E+000 |
| ‘D-Erythrose 4-phosphate’ | 3.48E−03 | 1.60E+04 | 1.60E+004 |
| ‘D-erythro-1-(Imidazol-4-yl)glycerol 3-phosphate’ | 6.42E−08 | 1.04E+06 | 1.04E+006 |
| ‘Enterochelin’ | 8.89E−07 | 1.19E+05 | 1.19E+005 |
| ‘D-Fructose 1-phosphate’ | 1.00E+00 | 1.29E+05 | 1.29E+005 |
| ‘ferric 2,3-dihydroxybenzoylserine’ | 6.42E−08 | 4.79E+01 | 4.79E+001 |
| ‘Fe(III)hydoxamate, unloaded’ | 5.33E−06 | 8.05E+04 | 8.05E+004 |
| ‘Ferrichrome minus Fe(III)’ | 5.33E−06 | 6.00E+00 | 6.00E+000 |
| ‘ferroxamine minus Fe(3)’ | 5.33E−06 | 7.27E+04 | 7.27E+004 |
| ‘2-(Formamido)-N1-(5-phospho-D-ribosyl)acetamidine’ | 2.00E+00 | 3.62E+05 | 3.62E+005 |
| ‘Glycerophosphoserine’ | 1.18E−08 | 1.04E+06 | 1.04E+006 |
| ‘galactosyl-glucosyl-inner core oligosaccharide lipid A’ | 4.00E+00 | 4.00E+00 | 0.00E+000 |
| ‘D-Galactonate’ | 6.42E−08 | 4.53E+05 | 4.53E+005 |
| ‘GDP-D-mannose’ | 1.18E−08 | 1.00E+00 | 1.00E+000 |
| ‘Guanosine 3″-diphosphate 5″-triphosphate’ | 1.18E−08 | 1.00E+00 | 1.00E+000 |
| ‘glucosyl-galactosyl-glucosyl-inner core oligosaccharide lipid A’ | 4.00E+00 | 4.00E+00 | 0.00E+000 |
| ‘glucosyl-inner core oligosaccharide lipid A’ | 4.00E+00 | 4.00E+00 | 0.00E+000 |
| ‘L-Glutamine’ | 6.66E−03 | 1.04E+06 | 1.04E+006 |
| ‘L-Glutamate’ | 1.00E+00 | 2.15E+04 | 2.15E+004 |
| ‘Glycerol 2-phosphate’ | 1.18E−08 | 8.93E+04 | 8.93E+004 |
| ‘Glutaryl-[acyl-carrier protein] methyl ester’ | 1.00E+00 | 1.00E+00 | 0.00E+000 |
| ‘D-Glycero-D-manno-heptose 1,7-bisphosphate’ | 1.89E−07 | 1.60E+01 | 1.60E+001 |
| ‘D-Glycero-D-manno-heptose 7-phosphate’ | 1.60E+01 | 1.60E+01 | 0.00E+000 |
| ‘Geranyl diphosphate’ | 4.91E+03 | 4.91E+03 | 2.98E−006 |
| ‘GTP’ | 6.42E−08 | 8.92E+01 | 8.92E+001 |
| ‘Glutathionylspermidine’ | 1.18E−08 | 1.00E+00 | 1.00E+000 |
| ‘H+’ | 5.71E−06 | 6.31E+05 | 6.31E+005 |
| ‘H2O’ | 2.52E+01 | 8.92E+01 | 6.40E+001 |
| ‘cis-hexadec-9-enoyl-[acyl-carrier protein] (n-C16:1)’ | 2.00E+00 | 4.87E+03 | 4.87E+003 |
| ‘3-Hydroxyglutaryl-[acyl-carrier protein] methyl ester’ | 6.42E−08 | 1.00E+00 | 1.00E+000 |
| ‘heptosyl-heptosyl-kdo2-lipidA’ | 4.00E+00 | 4.00E+00 | 0.00E+000 |
| ‘L-Histidinol phosphate’ | 8.96E−08 | 9.21E+03 | 9.21E+003 |
| ‘2-Hydroxy-6-oxonona-2,4-diene-1,9-dioate’ | 2.36E−08 | 9.03E+05 | 9.03E+005 |
| ‘2-hydroxy-6-ketononatrienedioate’ | 1.18E−08 | 2.40E+05 | 2.40E+005 |
| ‘Hydroxymethylbilane’ | 4.36E−06 | 2.42E+05 | 2.42E+005 |
| ‘L-Homoserine’ | 4.82E−07 | 1.78E+04 | 1.78E+004 |
| ‘heptosyl-phospho-heptosyl-heptosyl-kdo2-lipidA’ | 4.00E+00 | 4.00E+00 | 0.00E+000 |
| ‘3-Hydroxypimeloyl-[acyl-carrier protein] methyl ester’ | 6.42E−08 | 1.00E+00 | 1.00E+000 |
| ‘Iminoaspartate’ | 2.92E−07 | 2.25E+02 | 2.25E+002 |
| ‘Isochorismate’ | 4.30E−05 | 4.46E+01 | 4.46E+001 |
| ‘inner core oligosaccharide lipid A (E. coli)’ | 4.00E+00 | 4.00E+00 | 0.00E+000 |
| ‘3-(Imidazol-4-yl)-2-oxopropyl phosphate’ | 1.18E−06 | 5.18E+05 | 5.18E+005 |
| ‘Isopentenyl diphosphate’ | 4.86E+00 | 2.76E+03 | 2.75E+003 |
| ‘KDO(2)-lipid IV(A)’ | 1.00E +00 | 4.20E+04 | 4.20E+004 |
| ‘3-Deoxy-D-manno-octulosonate 8-phosphate’ | 3.32E−06 | 6.48E+03 | 6.48E+003 |
| ‘(R)-S-Lactoylglutathione’ | 8.96E−08 | 1.83E+06 | 1.83E+006 |
| ‘cold adapted KDO(2)-lipid (A)’ | 1.00E+00 | 3.29E+04 | 3.29E+004 |
| ‘Lipid A Disaccharide’ | 9.00E+00 | 4.20E+04 | 4.20E+004 |
| ‘lipoyl-AMP’ | 6.42E−08 | 1.00E+00 | 1.00E+000 |
| ‘Lipoate’ | 1.00E+00 | 1.00E+00 | 0.00E+000 |
| ‘L-Lyxose’ | 6.42E−08 | 7.00E+04 | 7.00E+004 |
| ‘malonyl-CoA methyl ester’ | 1.00E+00 | 1.00E+00 | 0.00E+000 |
| ‘2(alpha-D-Mannosyl-6-phosphate)-D-glycerate’ | 1.18E−08 | 1.05E+03 | 1.05E+003 |
| ‘(2R,4S)-2-methyl-2,4-dihydroxydihydrofuran-3-one’ | 1.60E−06 | 3.50E+05 | 3.50E+005 |
| ‘5,10-Methenyltetrahydrofolate’ | 8.96E−08 | 2.59E+04 | 2.59E+004 |
| ‘(2R,4S)-2-methyl-2,3,3,4-tetrahydroxytetrahydrofuran’ | 8.72E−06 | 3.50E+05 | 3.50E+005 |
| ‘Nicotinamide adenine dinucleotide’ | 1.18E−08 | 1.43E+07 | 1.43E+007 |
| ‘2-Oxopent-4-enoate’ | 3.54E−08 | 9.03E+05 | 9.03E+005 |
| ‘Oxamate’ | 9.24E−08 | 1.65E+06 | 1.65E+006 |
| ‘phosphatidylethanolamine (didodecanoyl, n-C12:0)’ | 2.00E+00 | 8.30E+04 | 8.30E+004 |
| ‘phosphatidylethanolamine (ditetradecanoyl, n-C14:0)’ | 2.00E+00 | 1.19E+06 | 1.19E+006 |
| ‘phosphatidylethanolamine (ditetradec-7-enoyl, n-C14:1)’ | 2.00E+00 | 5.23E+05 | 5.23E+005 |
| ‘phosphatidylethanolamine (dioctadecanoyl, n-C18:0)’ | 2.00E+00 | 1.18E+06 | 1.18E+006 |
| ‘Phosphoenolpyruvate’ | 1.00E−08 | 1.39E+06 | 1.39E+006 |
| ‘Phosphatidylglycerophosphate (didodecanoyl, n-C12:0)’ | 1.00E+00 | 6.21E+04 | 6.21E+004 |
| ‘Phosphatidylglycerophosphate (ditetradecanoyl, n-C14:0)’ | 1.00E+00 | 3.00E+00 | 2.00E+000 |
| ‘Phosphatidylglycerophosphate (ditetradec-7-enoyl, n-C14:1)’ | 1.00E+00 | 1.00E+01 | 9.00E+000 |
| ‘Phosphatidylglycerophosphate (dihexadecanoyl, n-C16:0)’ | 1.00E+00 | 1.94E+03 | 1.94E+003 |
| ‘Phosphatidylglycerophosphate (dihexadec-9-enoyl, n-C16:1)’ | 1.00E+00 | 4.37E+05 | 4.37E+005 |
| ‘Phosphatidylglycerophosphate (dioctadecanoyl, n-C18:0)’ | 1.00E+00 | 9.06E+04 | 9.06E+004 |
| ‘Phosphatidylglycerophosphate (dioctadec-11-enoyl, n-C18:1)’ | 1.00E+00 | 5.82E+04 | 5.82E+004 |
| ‘Protoheme’ | 1.00E+00 | 2.42E+05 | 2.42E+005 |
| ‘O-Phospho-L-homoserine’ | 1.18E−08 | 2.47E+04 | 2.47E+004 |
| ‘Pimeloyl-[acyl-carrier protein]’ | 2.66E−04 | 6.80E+02 | 6.80E+002 |
| ‘Pimeloyl-[acyl-carrier protein] methyl ester’ | 1.18E−08 | 1.00E+00 | 1.00E+000 |
| ‘(R)-Pantothenate’ | 1.59E+02 | 1.59E+02 | 1.53E−008 |
| ‘N-(5-Phospho-D-ribosyl)anthranilate’ | 1.80E−07 | 5.52E+03 | 5.52E+003 |
| ‘1-(5-Phosphoribosyl)-AMP’ | 8.96E−08 | 9.21E+03 | 9.21E+003 |
| ‘1-(5-Phosphoribosyl)-ATP’ | 8.96E−08 | 9.21E+03 | 9.21E+003 |
| ‘L-Prolinylglycine’ | 1.18E−08 | 2.15E+04 | 2.15E+004 |
| ‘5-Phospho-alpha-D-ribose 1-diphosphate’ | 9.28E−01 | 9.61E+05 | 9.61E+005 |
| ‘O-Phospho-L-serine’ | 1.25E−07 | 1.49E+06 | 1.49E+006 |
| ‘Putrescine’ | 4.66E−05 | 1.17E+06 | 1.17E+006 |
| ‘D-Ribose 1,5-bisphosphate’ | 1.00E+00 | 1.02E+07 | 1.02E+007 |
| ‘alpha-D-Ribose 1-phosphate’ | 1.00E+00 | 1.02E+07 | 1.02E+007 |
| ‘S-Ribosyl-L-homocysteine’ | 8.72E−06 | 3.50E+05 | 3.50E+005 |
| ‘D-Ribulose 5-phosphate’ | 5.73E−06 | 8.92E+01 | 8.92E+001 |
| ‘Sedoheptulose 7-phosphate’ | 1.03E−06 | 1.60E+01 | 1.60E+001 |
| ‘Sucrose 6-phosphate’ | 1.18E−08 | 8.07E+02 | 8.07E+002 |
| ‘Succinyl-CoA’ | 9.01E−08 | 1.19E+06 | 1.19E+006 |
| ‘N2-Succinyl-L-glutamate’ | 1.18E−08 | 1.47E+06 | 1.47E+006 |
| ‘N2-Succinyl-L-glutamate 5-semialdehyde’ | 2.23E−05 | 1.97E+06 | 1.97E+006 |
| ‘O-Succinyl-L-homoserine’ | 2.16E−06 | 1.56E+04 | 1.56E+004 |
| ‘D-tartrate’ | 6.42E−08 | 5.82E+05 | 5.82E+005 |
| ‘cis-tetradec-7-enoyl-[acyl-carrier protein] (n-C14:1)’ | 2.00E+00 | 1.88E+04 | 1.88E+004 |
| ‘Thiamin’ | 1.00E+00 | 2.13E+01 | 2.03E+001 |
| ‘Trehalose’ | 1.18E−08 | 5.40E+04 | 5.40E+004 |
| ‘alpha,alpha″-Trehalose 6-phosphate’ | 1.18E−08 | 5.40E+04 | 5.40E+004 |
| ‘tRNA (Glu)’ | 2.53E−02 | 1.94E+06 | 1.94E+006 |
| ‘UDP-2,3-bis(3-hydroxytetradecanoyl)glucosamine’ | 1.36E−06 | 3.24E+03 | 3.24E+003 |
| ‘UDP-3-O-(3-hydroxytetradecanoyl)-N-acetylglucosamine’ | 2.71E−06 | 6.48E+03 | 6.48E+003 |
| ‘UDP-3-O-(3-hydroxytetradecanoyl)-D-glucosamine’ | 1.47E−04 | 4.20E+04 | 4.20E+004 |
| ‘undecaprenyl phosphate-4-amino-4-formyl-L-arabinose’ | 1.18E−08 | 1.00E+00 | 1.00E+000 |
| ‘undecaprenyl phosphate-4-amino-4-deoxy-L-arabinose’ | 6.42E−08 | 1.00E+00 | 1.00E+000 |
| ‘Undecaprenyl-diphospho-N-acetylmuramoyl-(N-acetylglucosamine)- | 8.44E−05 | 1.54E+02 | 1.54E+002 |
| L-ala-D-glu-meso-2,6-diaminopimeloyl-D-ala-D-ala’ | | | |
| ‘Undecaprenyl-diphospho-N-acetylmuramoyl-L-alanyl-D-glutamyl- | 5.53E+02 | 3.51E+05 | 3.50E+005 |
| meso-2,6-diaminopimeloyl-D-alanyl-D-alanine’ | | | |
| ‘Undecaprenyl phosphate’ | 8.00E−01 | 8.00E−01 | 0.00E+000 |
| ‘UDP-N-acetylmuramoyl-L-alanyl-D-gamma-glutamyl-meso-2,6- | 1.29E−07 | 3.61E+04 | 3.61E+004 |
| diaminopimelate-D-alanine’ | | | |
| ‘Undecaprenyl-diphospho N-acetylglucosamine-N- | 2.57E−07 | 4.00E+00 | 4.00E+000 |
| acetylmannosaminuronate-N-acetamido-4,6-dideoxy-D-galactose’ | | | |
| ‘Ureidoacrylate peracid’ | 1.18E−08 | 5.93E+05 | 5.93E+005 |
| ‘Xanthine’ | 2.23E−05 | 3.99E+06 | 3.99E+006 |
| ‘XTP’ | 1.18E−08 | 2.00E+00 | 2.00E+000 |

The predicted ranges vary by several orders of magnitude for the metabolites with SCC (FIG. 3). We find that eighteen cytosolic metabolites are stringently constrained with a ratio between the upper and lower bounds of at least 0.95 (see FIG. 3, Table 2). These metabolites include: 6-hydroxymethyl dihydropterin, involved in the essential folate biosynthesis of E. coli, since this organism lacks uptake systems for folate cofactors (Bermingham, A. & Derrick, J. P. The folic acid biosynthesis pathway in bacteria: evaluation of potential for antibacterial drug discovery. BioEssays: news and reviews in molecular, cellular and developmental biology 24, 637-648, doi: 10.1002/bies.10114 (2002)) and 4-(cytidine 5′-diphospho)-2-C-methyl-D-erythritol, an isoprenoid precursor essential in eubacteria (Odom, A. R. Five questions about non-mevalonate isoprenoid biosynthesis. PLOS pathogens 7, e1002323, doi: 10.1371/journal.ppat.1002323 (2011)). Furthermore, the concentration of dephospho-CoA, a precursor of the essential coenzyme A required in the formation of key intermediates in energy metabolism (Sibon, O. C. & Strauss, E. Coenzyme A: to make it or uptake it? Nature reviews. Molecular cell biology 17, 605-606, doi: 10.1038/nrm.2016.110 (2016)) is kept in a narrow range under the analysed conditions. For energy-related metabolites we observe that ATP and NAD, even though they do exhibit SCC, have large ranges over the studied 17 conditions.

We compare the predicted ranges with a large experimental data set of absolute metabolite concentrations in E. coli under three different carbon sources (Bennett, B. D. et al. Absolute metabolite concentrations and implied enzyme active site occupancy in Escherichia coli. Nature chemical biology 5, 593-599, doi: 10.1038/nchembio. 186 (2009)). Of the 199 metabolites with SCC, eight were measured with glucose, glycerol, or acetate as only carbon source, respectively, and additional four were measured only with glucose as carbon source, yielding 28 measured concentrations for twelve metabolites (FIG. 4). The predicted large ranges for these metabolites result from the consideration of flux distributions fitted to 17 different experimental conditions and the ratios of relevant rate constants (see Example 1).

Out of these measured concentrations, 23 fall within the predicted ranges, while five (i.e., Glutamate with any of the three carbon sources, Adenosine phosphate and ADP-glucose with glucose as carbon source) were measured below the predicted ranges (FIG. 4). The reasons for the discrepancy include the combination of several factors: the differences in culture conditions used for the flux estimations (Nanchen, A., Schicker, A. & Sauer, U. Nonlinear dependency of intracellular fluxes on growth rate in miniaturized continuous cultures of Escherichia coli. Applied and environmental microbiology 72, 1164-1172, doi: 10.1128/AEM.72.2.1164-1172.2006 (2006)) and metabolite measurements (Bennett, B. D. et al. Absolute metabolite concentrations and implied enzyme active site occupancy in Escherichia coli. Nature chemical biology 5, 593-599, doi: 10.1038/nchembio.186 (2009)), such as dramatically different culture vessels (i.e., chemostat vs. filter culture), glucose concentrations (i.e., 4 g/L vs. 1 g/L) and growth rates (up to 0.4/hour vs. ˜0.6/hour). The differences may also be attributed to the inability to distinguish the concentrations of free metabolites from those bound to macromolecules experimentally (Bennett, B. D. et al. Absolute metabolite concentrations and implied enzyme active site occupancy in Escherichia coli. Nature chemical biology 5, 593-599, doi: 10.1038/nchembio. 186 (2009)), lack of crucial data on rate constants for reactions around these SCC metabolites, or model inaccuracies.

In the following, we inspect Eq. (1) to determine factors which may be responsible for the large predicted ranges. The reason for large predicted ranges can be the ratio of fluxes, vₚ/vₛ⁻ⁱ, the ratio of relevant rate constants which enter into the sums σp and σₛ⁻ⁱ, or the combination thereof. Note that the used values for the relevant rate constants are obtained from literature and span over 13 orders of magnitude (see Example 1). To investigate the effect of possible extremes for the relevant rate constants, we remove values which fall above or below different selected thresholds. In addition, several reactions were assigned a flux close to zero during the flux fitting procedure, which leads to very large flux ratios. Since such low fluxes are unlikely to be physiologically meaningful, we consider only those fluxes above a given threshold value. The support for this strategy comes from the fact that some reactions are usually inactive under specific environments, in which case their predicted low fluxes can be neglected (Robaina Estevez, S. & Nikoloski, Z. Generalized framework for context-specific metabolic model extraction methods. Frontiers in plant science 5, 491, doi: 10.3389/fpls.2014.00491 (2014)). By using these strategies, we find that for 11 of the 12 measured metabolites with SCC, the predicted ranges were reduced by up to 10 orders of magnitude (FIG. 9, Table 3). We find that the rate constants have smaller effect on the predicted concentration ranges in comparison to the flux ratios.

TABLE 3
Concentration range for 12 cytosolic metabolites predicted from a genome-scale metabolic
model of E. coli under different constraints.
The concentration range is given in mmol/gDW. Under some constraints no concentration
could be predicted since the prediction only relied on
reactions not considered under the additional constraint. See also FIG. 4 and FIG. 9.

| | | | max fraction | flux | flux | flux | max |
|---|---|---|---|---|---|---|---|
| | | | of range | threshold | threshold | threshold | fraction of |
| predicted | predicted | predicted | reduction by | 10\hat{ }-4 | 10\hat{ }-3 | 10\hat{ }-2 | range reduction |
| range no | range 5% | range 10% | truncation of | predicted | predicted | predicted | by threshold on |
| constraints | truncation | truncation | rate constants | range | range | range | reaction flux |
| 2.15E+004 | 2.15E+004 | 2.15E+004 | 1.00E+000 | 2.15E+004 | NaN | NaN | 1.00E+000 |
| 1.00E+008 | 1.00E+008 | 1.00E+008 | 1.00E+000 | 1.07E+004 | 9.85E+002 | 9.85E+002 | 1.02E+005 |
| 1.00E+008 | 1.00E+008 | 1.00E+008 | 1.00E+000 | 1.00E+000 | 3.42E−003 | NaN | 2.92E+010 |
| 1.43E+007 | 1.43E+007 | 1.43E+007 | 1.00E+000 | 1.22E+007 | 8.56E+002 | 8.56E+002 | 1.67E+004 |
| 1.04E+006 | 1.04E+006 | 1.04E+006 | 1.00E+000 | 6.60E+005 | 5.83E+002 | 2.91E−008 | 3.56E+013 |
| 3.34E+005 | 3.34E+005 | 3.51E+006 | 1.00E+000 | 3.34E+005 | 1.51E+002 | 1.51E+002 | 2.22E+003 |
| 1.97E+006 | 1.97E+006 | 1.97E+006 | 1.00E+000 | 1.43E+001 | 1.43E+001 | 1.43E+001 | 1.38E+005 |
| 6.55E+005 | 6.55E+005 | 6.55E+005 | 1.00E+000 | 4.29E+005 | 1.92E+002 | 5.84E−001 | 1.12E+006 |
| 1.39E+006 | 1.39E+006 | 1.39E+006 | 1.00E+000 | 5.82E+005 | 1.14E+001 | 1.14E+001 | 1.22E+005 |
| 1.00E+000 | 1.00E+000 | 1.00E+000 | 1.00E+000 | 2.13E−007 | NaN | NaN | 4.70E+006 |
| 1.23E+005 | 1.23E+005 | 1.23E+005 | 1.00E+000 | 3.04E+002 | NaN | NaN | 4.03E+002 |
| 1.54E+004 | 1.54E+004 | 1.54E+004 | 1.00E+000 | 1.54E+004 | 1.25E+003 | 1.03E−001 | 1.49E+005 |

| | | | | | | max fraction |
|---|---|---|---|---|---|---|
| | | | | | | of range on |
| | | | | | | reaction flux |
| | | | | | | and max fraction |
| predicted range | predicted range | predicted range | predicted range | predicted range | predicted range | of range reduction |
| flux threshold | flux threshold | flux threshold | flux threshold | flux threshold | flux threshold | by threshold |
| 10\hat{ }-4 and 5% | 10\hat{ }-4 and 10% | 10\hat{ }-3 and 5% | 10\hat{ }-3 and 10% | 10\hat{ }-2 and 5% | 10\hat{ }-2 and 10% | rate constant |
| truncation | truncation | truncation | truncation | truncation | truncation | truncation |
| 2.15E+004 | NaN | NaN | 2.15E+004 | NaN | NaN | 1.00E+000 |
| 1.07E+004 | 9.85E+002 | 9.85E+002 | 1.07E+004 | 9.85E+002 | 9.85E+002 | 1.02E+005 |
| 1.00E+000 | 3.42E−003 | NaN | 1.00E+000 | 3.42E−003 | NaN | 2.92E+010 |
| 1.22E+007 | 8.56E+002 | 8.56E+002 | 1.22E+007 | 8.56E+002 | 8.56E+002 | 1.67E+004 |
| 2.76E+003 | 2.43E+000 | 1.22E−010 | 2.76E+003 | 2.43E+000 | 1.22E−010 | 8.50E+015 |
| 3.34E+005 | 1.51E+002 | 1.51E+002 | 3.34E+005 | 1.51E+002 | 1.51E+002 | 2.22E+003 |
| 1.43E+001 | 1.43E+001 | 1.43E+001 | 1.43E+001 | 1.43E+001 | 1.43E+001 | 1.38E+005 |
| 4.29E+005 | 1.92E+002 | 5.84E−001 | 4.29E+005 | 1.92E+002 | 5.84E−001 | 1.12E+006 |
| 5.82E+005 | 1.14E+001 | 1.14E+001 | 5.82E+005 | 1.14E+001 | 1.14E+001 | 1.22E+005 |
| 2.13E−007 | NaN | NaN | 2.13E−007 | NaN | NaN | 4.70E+006 |
| 3.04E+002 | NaN | NaN | 3.04E+002 | NaN | NaN | 4.03E+002 |
| 1.54E+004 | 1.25E+003 | 1.03E−001 | 1.54E+004 | 1.25E+003 | 1.03E−001 | 1.49E+005 |

Due to the derivation of Eq. (1), our findings imply that the concentrations of metabolites with SCC can be readily controlled by manipulating only selected fluxes. The observation that the structurally constrained metabolites are involved in vital cellular processes in E. coli suggests that the particular network structure plays an essential role in enabling the molecular dynamics of viable cells and further highlights the tight interrelation between metabolite concentrations and reaction fluxes (Hackett, S. R. et al. Systems-level analysis of mechanisms regulating yeast metabolic flux. Science 354, doi: 10.1126/science.aaf2786 (2016); Davidi, D. et al. Global characterization of in vivo enzyme catalytic rates and their correspondence to in vitro kcat measurements. Proceedings of the National Academy of Sciences of the United States of America 113, 3401-3406, doi: 10.1073/pnas.1514240113 (2016)).

### Example 6

Components with SCC Across Species

We next apply Eq. (1) to 14 large-scale metabolic networks which differ in complexity due to the number of considered metabolites and reactions as well as their organization in subcellular compartments (Table 4). The investigated metabolic networks are mass- and charge-balanced and support positive steady-state reaction rates (see Example 1). Since reliable kinetic information is currently missing across diverse species, we report only the number of the metabolites with SCC across the analyzed large-scale networks.

TABLE 4
Number of metabolites with structurally constrained concentrations and absolute
concentration robustness for each of the metabolic networks analyzed. The numbers of
reactions and metabolites correspond to the number after reaction splitting into irreversible
reactions and removal of blocked reactions. The latter is needed to satisfy the prerequisite for
a positive steady state. All 14 networks are of structural deficiency greater than 1; therefore,
established methods for concentration robustness cannot be applied.

| | | | | | | | Number of |
|---|---|---|---|---|---|---|---|
| | | | | | | | ACR |
| | | | | | | | metabolites |
| | | | | | | | involved in |
| | | | | | | | network |
| | | Number of | Number of | | Number | % | motif of |
| Species | Model name | metabolites | reactions | deficiency | of ACR | of ACR | two reactions |
| A. niger | iAM871 | 887 | 1773 | 326 | 36 | 4.06 | 14.00 |
| A. thaliana | AraCORE | 407 | 675 | 117 | 0 | 0.00 | — |
| C. reinhardtii | iRC1080 | 162 | 301 | 53 | 0 | 0.00 | — |
| E. coli K12 | iJO1366 | 1602 | 3234 | 759 | 14 | 0.87 | 0.00 |
| H. sapiens | recon1 | 1587 | 3618 | 889 | 12 | 0.76 | 0.00 |
| M. acetivorans | iMB745 | 507 | 891 | 138 | 2 | 0.39 | 0.00 |
| M. barkeri | iMG746 | 509 | 879 | 131 | 1 | 0.20 | 0.00 |
| M. pneumoniae | iJW145 | 241 | 374 | 55 | 7 | 2.90 | 6.00 |
| N. pharaonis | iOG654 | 465 | 916 | 126 | 0 | 0.00 | — |
| P. putida | iJP962 | 595 | 1015 | 167 | 0 | 0.00 | — |
| S. aureus | iSB619 | 449 | 821 | 128 | 4 | 0.89 | 0.00 |
| Synechocystis sp. | iJN678 | 633 | 948 | 111 | 0 | 0.00 | — |
| T. maritima | iTZ479 | 429 | 784 | 122 | 2 | 0.4 | 0.00 |
| Y. pestis | iPC815 | 761 | 1348 | 320 | 0 | 0.00 | — |
| H. sapiens | liver | 4294 | 12488 | 3713 | 32 | 0.75 | 2.00 |
| H. sapiens | iLiverCancer1715 | 3723 | 9750 | 2429 | 36 | 0.97 | 4.00 |
| H. sapiens | lung | 4285 | 12441 | 3680 | 31 | 0.72 | 3.00 |
| H. sapiens | iLungCancer1472 | 3371 | 8721 | 2156 | 40 | 1.19 | 4.00 |
| H. sapiens | kidney | 4332 | 12875 | 3903 | 34 | 0.78 | 4.00 |
| H. sapiens | iRenalCancer1410 | 3395 | 8838 | 2166 | 40 | 1.18 | 1.00 |
| H. sapiens | urinary | 4268 | 12197 | 3565 | 33 | 0.77 | 3.00 |
| H. sapiens | iUrothelialCancer1647 | 3570 | 9520 | 2404 | 42 | 1.18 | 5.00 |

| | | Number of | % of ACR | | |
|---|---|---|---|---|---|
| | | ACR metabolites | metabolites of | Number of | % of |
| | | involved in network | metabolites | structurally | structurally |
| | | motif of three | exchanged with | constrained | constrained |
| Species | Model name | reactions | the environment | metabolites | metabolites |
| A. niger | iAM871 | 22.00 | 23.29 | 134 | 15.11 |
| A. thaliana | AraCORE | — | 0.00 | 137 | 33.66 |
| C. reinhardtil | iRC1080 | — | 0.00 | 13 | 8.02 |
| E. coli K12 | iJO1366 | 14.00 | 4.61 | 367 | 22.91 |
| H. sapiens | recon1 | 11.00 | 3.52 | 306 | 19.28 |
| M. acetivorans | iMB745 | 2.00 | 2.08 | 68 | 13.41 |
| M. barkeri | iMG746 | 1.00 | 1.82 | 71 | 13.95 |
| M. pneumoniae | iJW145 | 1.00 | 13.46 | 25 | 10.37 |
| N. pharaonis | iOG654 | — | 0.00 | 36 | 7.74 |
| P. putida | iJP962 | — | 0.00 | 93 | 15.63 |
| S. aureus | iSB619 | 3.00 | 4.29 | 55 | 12.25 |
| Synechocystis sp. | iJN678 | — | 0.00 | 135 | 21.33 |
| T. maritima | iTZ479 | 2.00 | 1.27 | 74 | 17.25 |
| Y. pestis | iPC815 | — | 0.00 | 278 | 36.53 |
| H. sapiens | liver | 28.00 | 1.19 | 860 | 20.03 |
| H. sapiens | iLiverCancer1715 | 16.00 | 1.43 | 866 | 23.26 |
| H. sapiens | lung | 25.00 | 1.12 | 787 | 18.37 |
| H. sapiens | iLungCancer1472 | 32.00 | 1.74 | 717 | 21.27 |
| H. sapiens | kidney | 27.00 | 1.13 | 683 | 15.77 |
| H. sapiens | iRenalCancer1410 | 35.00 | 1.88 | 726 | 21.38 |
| H. sapiens | urinary | 27.00 | 1.21 | 793 | 18.58 |
| H. sapiens | iUrothelialCancer1647 | 33.00 | 1.67 | 746 | 20.90 |
| | | | % structurally | % structurally | |
| | | % structurally | constrained | constrained | % structurally |
| | | constrained | metabolites with | metabolites with | constrained |
| | | metabolites with | degree >2 | degree >5 | metabolites with |
| Species | Model name | degree <= 2 | and <= 5 | and <= 10 | degree >10 |
| A. niger | iAM871 | 48.51 | 37.31 | 4.48 | 9.70 |
| A. thaliana | AraCORE | 49.64 | 31.39 | 14.60 | 4.38 |
| C. reinhardtil | iRC1080 | 38.46 | 30.77 | 23.08 | 7.69 |
| E. coli K12 | iJO1366 | 56.13 | 33.79 | 5.99 | 4.09 |
| H. sapiens | recon1 | 58.17 | 23.53 | 9.48 | 8.82 |
| M. acetivorans | iMB745 | 51.47 | 23.53 | 7.35 | 17.65 |
| M. barkeri | iMG746 | 56.34 | 22.54 | 7.04 | 14.08 |
| M. pneumoniae | iJW145 | 68.00 | 20.00 | 8.00 | 4.00 |
| N. pharaonis | iOG654 | 61.11 | 16.67 | 8.33 | 13.89 |
| P. putida | iJP962 | 62.37 | 23.66 | 6.45 | 7.53 |
| S. aureus | iSB619 | 50.91 | 30.91 | 5.45 | 12.73 |
| Synechocystis sp. | iJN678 | 71.11 | 14.07 | 5.19 | 9.63 |
| T. maritima | iTZ479 | 43.24 | 41.89 | 5.41 | 9.46 |
| Y. pestis | iPC815 | 37.05 | 50.00 | 10.43 | 2.52 |
| H. sapiens | liver | 63.59 | 22.08 | 3.46 | 10.87 |
| H. sapiens | iLiverCancer1715 | 64.58 | 13.34 | 11.53 | 10.54 |
| H. sapiens | lung | 65.27 | 17.48 | 4.42 | 12.83 |
| H. sapiens | iLungCancer1472 | 68.14 | 15.49 | 3.98 | 12.39 |
| H. sapiens | kidney | 64.86 | 17.46 | 4.37 | 13.31 |
| H. sapiens | iRenalCancer1410 | 66.53 | 16.84 | 3.95 | 12.68 |
| H. sapiens | urinary | 65.61 | 17.20 | 4.25 | 12.95 |
| H. sapiens | iUrothelialCancer1647 | 66.67 | 16.35 | 3.82 | 13.16 |

We find that the percentage of metabolites with SCC ranges from 7.74% and 8.02% in the models of N. pharaonis and C. reinhardtii to 33.66% and 36.53% in the models of A. thaliana and Y. pestis (FIG. 5a). Interestingly, the number of metabolites with SCC scales linearly with the total number of metabolites (FIG. 5b, R²=0.82) and the number of reactions in the examined networks (FIG. 5c, R²=0.76). This finding indicates that the proposed approach is not limited to networks of a particular size.

Different reasons can be used to explain the observation that larger networks contain more metabolites with SCC. For instance, larger networks may include more linear pathways, whereby the number of reactions which are fully coupled due to structure is expected to increase. Yet, in denser networks, which include more reactions on the same set of metabolites, it is more likely to identify reactions which share substrates of same stoichiometry, which then leads to full coupling due to mass action kinetics, as considered in our approach. To investigate the reasons for the scaling of the number of metabolites with SCC, we determine the number of: (i) metabolites which are synthesized and used by one reaction, respectively (in support of the linear pathway explanation), (ii) fully coupled reactions only due to structure, (iii) coupled reactions due to mass action (in support of the network density explanation), (iv) the combination of (ii) and (iii), to assess the couplings due to both structure and kinetics (Table 5). We calculate the Pearson correlation coefficient between each of these properties and the number of reactions over the analyzed networks, as a measure of network size (Table 5). Larger networks indeed contain a bigger number of metabolites synthesized and used by a single reaction, respectively, and more reactions which are fully coupled due to both structure and kinetics. Therefore, both the linear pathway and the network density explanations contribute to the observed scaling in the analyzed networks.

TABLE 5
Fraction of fully coupled reactions and reactions coupled due to mass action kinetics
in 14 analyzed genome-scale metabolic networks.

| | | | | | Number of | |
|---|---|---|---|---|---|---|
| | | | | | metabolites which | % of metabolites |
| | | | | | are synthesized | are synthesized |
| | | | Number of | Number of | and used by | used by a single |
| Kingdom | Species | Model name | metabolites | reactions | a single reaction | reaction |
| plantae | C. reinhardtii | iRC1080 | 162 | 301 | 13 | 8.02 |
| eubacteria | M. pneumoniae | iJW145 | 241 | 374 | 44 | 18.26 |
| plantae | A. thaliana | AraCORE | 407 | 675 | 141 | 34.64 |
| eubacteria | T. maritima | iTZ479 | 429 | 784 | 98 | 22.84 |
| eubacteria | S. aureus | iSB619 | 449 | 821 | 99 | 22.05 |
| euryarchaeota | M. barkeri | iMG746 | 509 | 879 | 142 | 27.90 |
| euryarchaeota | M. acetivorans | iMB745 | 507 | 891 | 141 | 27.81 |
| eucaryota | N. pharaonis | iOG654 | 465 | 916 | 74 | 15.91 |
| bacteria | Synechocystis sp. | iJN678 | 633 | 948 | 207 | 32.70 |
| eubacteria | P. putida | iJP962 | 595 | 1015 | 201 | 33.78 |
| eubacteria | Y. pestis | iPC815 | 761 | 1348 | 203 | 26.68 |
| fungi | A. niger | iAM871 | 887 | 1773 | 145 | 16.35 |
| eubacteria | E. coli K12 | iJO1366 | 1602 | 3234 | 336 | 20.97 |
| animalia | H. sapiens | recon1 | 1587 | 3618 | 290 | 18.27 |
| | | | | | Number of | % of fully |
| | | | | | fully coupled | coupled |
| | | | | | reaction | reaction pairs |
| | | | Number of | % of fully | pairs (coupling | (coupling to |
| | | | fully coupled | coupled | to itself | itself |
| Kingdom | Species | Model name | reactions | reactions | excluded) | excluded) |
| plantae | C. reinhardtii | iRC1080 | 14 | 4.65 | 19 | 0.04 |
| eubacteria | M. pneumoniae | iJW145 | 47 | 12.57 | 231 | 0.33 |
| plantae | A. thaliana | AraCORE | 144 | 21.33 | 505 | 0.22 |
| eubacteria | T. maritima | iTZ479 | 126 | 16.07 | 816 | 0.27 |
| eubacteria | S. aureus | iSB619 | 125 | 15.23 | 715 | 0.21 |
| euryarchaeota | M. barkeri | iMG746 | 179 | 20.36 | 1935 | 0.50 |
| euryarchaeota | M. acetivorans | iMB745 | 176 | 19.75 | 2159 | 0.54 |
| eucaryota | N. pharaonis | iOG654 | 94 | 10.26 | 392 | 0.09 |
| bacteria | Synechocystis sp. | iJN678 | 285 | 30.06 | 2437 | 0.54 |
| eubacteria | P. putida | iJP962 | 213 | 20.99 | 2049 | 0.40 |
| eubacteria | Y. pestis | iPC815 | 242 | 17.95 | 571 | 0.06 |
| fungi | A. niger | iAM871 | 174 | 9.81 | 1925 | 0.12 |
| eubacteria | E. coli K12 | iJO1366 | 382 | 11.81 | 1124 | 0.02 |
| animalia | H. sapiens | recon1 | 320 | 8.84 | 582 | 0.01 |
| | | | Number of | % of of | Number of | % of reaction |
| | | | reactions | reactions | reaction pairs | pairs coupled |
| | | | coupled | coupled due | coupled due to | due to |
| | | | due to mass | to mass | mass action | mass action |
| Kingdom | Species | Model name | action kinetics | action kinetics | kinetics | kinetics |
| plantae | C. reinhardtii | iRC1080 | 38 | 12.62 | 38 | 0.08 |
| eubacteria | M. pneumoniae | iJW145 | 11 | 2.94 | 11 | 0.02 |
| plantae | A. thaliana | AraCORE | 72 | 10.67 | 72 | 0.03 |
| eubacteria | T. maritima | iTZ479 | 27 | 3.44 | 27 | 0.01 |
| eubacteria | S. aureus | iSB619 | 27 | 3.29 | 27 | 0.01 |
| euryarchaeota | M. barkeri | iMG746 | 52 | 5.92 | 52 | 0.01 |
| euryarchaeota | M. acetivorans | iMB745 | 45 | 5.05 | 45 | 0.01 |
| eucaryota | N. pharaonis | iOG654 | 32 | 3.49 | 32 | 0.01 |
| bacteria | Synechocystis sp. | iJN678 | 66 | 6.96 | 66 | 0.01 |
| eubacteria | P. putida | iJP962 | 27 | 2.66 | 27 | 0.01 |
| eubacteria | Y. pestis | iPC815 | 126 | 9.35 | 126 | 0.01 |
| fungi | A. niger | iAM871 | 172 | 9.70 | 172 | 0.01 |
| eubacteria | E. coli K12 | iJO1366 | 395 | 12.21 | 395 | 0.01 |
| animalia | H. sapiens | recon1 | 339 | 9.37 | 339 | 0.01 |
| | | | Number of fully | | % of fully coupled | |
| | | | coupled reactions | | reaction pairs and | |
| | | | and reactions | | reaction pairs | |
| | | | coupled due to | | coupled due to | |
| | | | mass action | | mass action | |
| Kingdom | Species | Model name | kinetics | | kinetics | |
| plantae | C. reinhardtii | iRC1080 | 57 | | 0.13 | |
| eubacteria | M. pneumoniae | iJW145 | 242 | | 0.35 | |
| plantae | A. thaliana | AraCORE | 577 | | 0.25 | |
| eubacteria | T. maritima | iTZ479 | 841 | | 0.27 | |
| eubacteria | S. aureus | iSB619 | 742 | | 0.22 | |
| euryarchaeota | M. barkeri | iMG746 | 1987 | | 0.51 | |
| euryarchaeota | M. acetivorans | iMB745 | 2204 | | 0.56 | |
| eucaryota | N. pharaonis | iOG654 | 424 | | 0.10 | |
| bacteria | Synechocystis sp. | iJN678 | 2503 | | 0.56 | |
| eubacteria | P. putida | iJP962 | 2076 | | 0.40 | |
| eubacteria | Y. pestis | iPC815 | 697 | | 0.08 | |
| fungi | A. niger | iAM871 | 2097 | | 0.13 | |
| eubacteria | E. coli K12 | iJO1366 | 1519 | | 0.03 | |
| animalia | H. sapiens | recon1 | 921 | | 0.01 | |
| | Number of | | | | | Number of fully |
| | metabolites | | | | | coupled reactions |
| | which are | | Number of fully | | | and reactions |
| Number | synthesized and | | coupled reaction | Number of reactions | Number of reaction | coupled due to |
| of | used by a single | Number of fully | pairs (coupling to | coupled due to mass | pairs coupled due to | mass action |
| reactions | reaction | coupled reactions | itself excluded) | action kinetics | mass action kinetics | kinetics |
| | 0.85012 | 0.81151 | 0.051782 | 0.96295 | 0.96295 | 0.19215 |
| | (0.00011754) | (0.00042562) | (0.86045) | (3.4464e−08) | (3.4464e−08) | (0.51047) |

Due to the derivation of Eq. (1), it may be expected that the approach is difficult to apply to components which participate in a large number of reactions, since they may be less likely to be fully coupled. Nevertheless, our findings show that between 28.89% and 62.95% of the metabolites with structurally constrained concentrations in the analyzed networks are involved in more than two reactions (see Table 4). One reason is that a SCC metabolite may also be determined by applying Eq. (1) to the ODE of another metabolite (see Algorithm in Examples 1 and 2).

For essential metabolic processes to be carried out efficiently, metabolites that serve as coenzymes and energy currency of biological systems, namely, the oxidized and reduced version of NAD and NADP as well as the adenosine phosphates (i.e. AMP, ADP, ATP), are maintained within certain concentration ranges that can be readily controlled, as is the case for SCC metabolites. Despite the many biochemical reactions in which these ubiquitous metabolites participate (Table 6), all of which must satisfy our conditions in order to invoke Eq. (1), we find that the (sub) cellular concentrations of ATP and NAD are indeed structurally constrained in twelve and ten of the analyzed networks, respectively. This implies that the network structure, alongside a limited set of rate constants and a single flux ratio, imposes boundaries on and facilitates simple control over their concentrations. In addition, we find that NADP shows SCC in four of the investigated networks, including A. thaliana and C. reinhardtii (Table 6 and Table 7). In these photosynthetic organisms, NADPH is produced by ferredoxin-NADP+ reductase in the last step of the electron transport chain which constitute the light reactions of photosynthesis (Berg, J. M., Tymoczko, J. L. & Stryer, L. Biochemistry. 6 edn, (W. H. Freeman, 2007)). The produced NADPH provides reducing power for the biosynthetic reactions in the Calvin cycle to fix carbon dioxide as well as in the reduction of nitrate into ammonia for plant assimilation in the nitrogen cycle. Therefore, precise and simple control of NADPH will provide uninterrupted functionality of these key metabolic pathways and maintenance of carbon and nitrogen balance (Stitt, M. et al. Steps towards an integrated view of nitrogen metabolism. Journal of experimental botany 53, 959-970 (2002)). In addition, for ten models, we find that H+ has structurally constrained concentration ensuring maintenance of the specific functions of individual organelles (Casey, J. R., Grinstein, S. & Orlowski, J. Sensors and regulators of intracellular pH. Nature reviews. Molecular cell biology 11, 50-61, doi: 10.1038/nrm2820 (2010)).

TABLE 6
Structurally constrained metabolites across the 14 analyzed metabolic networks. In
addition, the in- and out-degree for these metabolites are provided. Metabolites marked in red
correspond to energy metabolism (see Table 1 in the main text) and metabolites marked in
green exhibit absolute concentration robustness. Metabolite names and their abbreviations are
used as provided in the original models.

| A. niger | in | out |
|---|---|---|
| iAM871 | degree | degree |
| ‘NAG--N-Acetyl-D-glucosamine’ | 2 | 1 |
| ‘3CMUCO--3-Carboxymuconate’ | 2 | 2 |
| ‘GPP--Geranyl diphosphate’ | 1 | 1 |
| HSER--L-Homoserine’ | 2 | 2 |
| ‘TDPE1m--(thiamine diphosphate)-alpha-ketoglutarate dehydrogenase’ | 1 | 1 |
| ‘MANe--D-Mannose’ | 1 | 1 |
| ‘3PSME--5-O-(1-Carboxyvinyl)-3-phosphoshikimate’ | 1 | 1 |
| ‘PCPS--Phosphatidylserine used for phosphatidylcholine’ | 1 | 2 |
| ‘PEPS--Phosphatidylserine used for phosphatidylethanolamine’ | 1 | 2 |
| ‘AKGm--2-Oxoglutarate’ | 11 | 11 |
| ‘LACTe--Lactose’ | 1 | 2 |
| ‘THRe--L-Threonine’ | 1 | 2 |
| ‘DMIPC--Di-mannosyl-inositol-P-ceramide’ | 1 | 1 |
| ‘SEMAG--Monoacylglycerol(for Sterolesters)’ | 1 | 1 |
| ‘IGP--Indoleglycerol phosphate’ | 1 | 1 |
| ‘2HPAC--2-hydroxyphenylacetic acid’ | 1 | 1 |
| ‘RLe--D-Ribulose’ | 1 | 1 |
| ‘S23E--(S)-2,3-Epoxysqualene’ | 1 | 1 |
| ‘EU--L-Erythrulose’ | 2 | 1 |
| ‘3DDAH7P--2-Dehydro-3-deoxy-D-arabino-heptonate 7-phosphate’ | 1 | 1 |
| ‘Ee--D-Erythrose’ | 1 | 1 |
| ‘CERB2A--Cerebrin 2(comp. A)’ | 1 | 2 |
| ‘CERB2B--Cerebrin 2(comp. B)’ | 1 | 2 |
| ‘PEP--Phosphoenolpyruvate’ | 2 | 4 |
| ‘OIVALm--(R)-2-Oxoisovalerate’ | 2 | 2 |
| ‘CYTS--Cytosine’ | 1 | 1 |
| ‘THRm--L-Threonine’ | 1 | 2 |
| ‘CDA--Cholesta-8,24-dien-3-ol-4-carboxylate’ | 1 | 1 |
| ‘CYSe--L-Cysteine’ | 1 | 2 |
| ‘MELle--Melibiose’ | 1 | 1 |
| ‘LARABe--L-Arabinose’ | 1 | 1 |
| ‘CLPIGP--3(3-sn-phosphatidyl)-sn-glycerol 1-phosphate used for cardiolipin | 1 | 1 |
| biosynthesis’ | | |
| ‘3PSER--3-Phosphoserine’ | 1 | 1 |
| ‘TRE6P--alpha, alpha-Trehalose 6-phosphate’ | 1 | 1 |
| ‘METe--L-Methionine’ | 1 | 2 |
| ‘HISe--L-Histidine’ | 1 | 2 |
| ‘NADP--NADP(+)’ | 112 | 70 |
| ‘C171COA--Heptadecenoyl-CoA’ | 2 | 2 |
| ‘PRFP--5-(5-Phospho-D-ribosylaminoformimino)-1-(5-phosphoribosyl)- | 1 | 1 |
| imidazole-4-carboxamide’ | | |
| ‘TRPe--L-Tryptophan’ | 1 | 2 |
| ‘PRBAMP--N1-(5-Phospho-D-ribosyl)-AMP’ | 1 | 1 |
| ‘PRCP--5-Phosphoribosyl monophosphate’ | 1 | 1 |
| ‘EUe--L-Erythrulose’ | 1 | 1 |
| ‘NADPH--NADPH’ | 68 | 112 |
| ‘SERe--L-Serine’ | 1 | 2 |
| ‘CMP--CMP’ | 8 | 7 |
| ‘F6P--beta-D-Fructose 6-phosphate’ | 9 | 9 |
| ‘MVL--(R)-Mevalonate’ | 1 | 2 |
| ‘SLF--Sulfate’ | 1 | 2 |
| ‘C18SPH--Sphinganine(C18)’ | 1 | 3 |
| ‘VALe--L-Valine’ | 1 | 2 |
| ‘ASUC--N6-(1,2-Dicarboxyethyl)-AMP’ | 1 | 1 |
| ‘CPAD5P--1-(2-Carboxyphenylamino)-1-deoxy-D-ribulose 5-phosphate’ | 1 | 1 |
| ‘IPPP--Isopentenyl diphosphate’ | 2 | 3 |
| ‘SME--Shikimate’ | 3 | 2 |
| ‘LXULe--L-Xylulose’ | 1 | 1 |
| ‘PROPALe--Propionaldehyde’ | 1 | 2 |
| ‘4CMUCL--4-Carboxymuconolactone’ | 1 | 2 |
| ‘LYSe--L-Lysine’ | 1 | 2 |
| ‘PRLP--N-(5″-Phospho-D-1″-ribulosylformimino)-5-amino-1-(5″-phospho-D- | 1 | 1 |
| ribosyl)-4-imidazolecarboxamide’ | | |
| ‘PHSER--O-Phospho-L-homoserine’ | 1 | 1 |
| ‘OTA--Ochratoxin A’ | 1 | 1 |
| ‘ASPe--L-Aspartate’ | 1 | 2 |
| ‘SQL--Squalene’ | 1 | 1 |
| ‘345THBe--Gallic acid’ | 2 | 1 |
| ‘NAD--NAD(+)’ | 41 | 54 |
| ‘PMVL--(R)-5-Phosphomevalonate’ | 1 | 1 |
| ‘ASNe--L-Asparagine’ | 1 | 2 |
| ‘D6PGL--d-Glucono-1,5-lactone 6-phosphate’ | 1 | 1 |
| ‘GTP--GTP’ | 2 | 6 |
| ‘MAN--D-Mannose’ | 1 | 1 |
| ‘THR--L-Threonine’ | 4 | 3 |
| ‘MI1P--1L-myo-Inositol 1-phosphate’ | 1 | 2 |
| ‘GDMIPC--Galactosyl-Dimannosyl-inositol-P-ceramide’ | 1 | 1 |
| ‘MLTe--Maltose’ | 1 | 2 |
| ‘ALAe--L-Alanine’ | 1 | 2 |
| ‘ARGe--L-Arginine’ | 1 | 2 |
| ‘ATP--ATP’ | 27 | 99 |
| ‘GLYe--Glycine’ | 1 | 2 |
| ‘ARABe--D-Arabinose’ | 1 | 1 |
| ‘cAMP--3″,5″-Cyclic AMP’ | 1 | 1 |
| ‘H2SO3e--Sulfite’ | 1 | 1 |
| ‘NPRAN--N-(5-Phospho-D-ribosyl)anthranilate″ | 1 | 1 |
| ‘PROe--L-Proline’ | 1 | 2 |
| ‘PRPP--5-Phospho-alpha-D-ribose 1-diphosphate’ | 2 | 13 |
| ‘GALUNTe--D-Galacturonate’ | 1 | 1 |
| ‘APS--Adenylylsulfate’ | 1 | 1 |
| ‘GLUe--L-Glutamate’ | 1 | 2 |
| ‘C180COA--Octadecanoyl-CoA’ | 3 | 8 |
| ‘GABAm--4-Aminobutanoate’ | 2 | 2 |
| ‘RIBe--D-Ribose’ | 1 | 1 |
| ‘ACCOA--Acetyl-CoA’ | 9 | 10 |
| ‘ASP--L-Aspartate’ | 6 | 12 |
| ‘LACT--Lactose’ | 1 | 1 |
| ‘DCDA--4-methylcholesta-8,24-diene-3-ol-4-carboxylate’ | 1 | 1 |
| ‘PPMVL--(R)-5-Diphosphomevalonate’ | 1 | 1 |
| ‘DIMGP--D-erythro-1-(Imidazol-4-yl)glycerol 3-phosphate’ | 1 | 1 |
| ‘HOMOGEN--Homogentisate’ | 3 | 2 |
| ‘DHVALm--(R)-2,3-dihydroxy-3-methylbutanoate’ | 1 | 1 |
| ‘NAORNm--N2-Acetyl-L-ornithine’ | 1 | 1 |
| ‘PRBATP--N1-(5-Phospho-D-ribosyl)-ATP’ | 1 | 1 |
| ‘FGAM--2-(Formamido)-N1-(5″-phosphoribosyl)acetamidine’ | 1 | 1 |
| ‘CEREB1--Cerebroside 1’ | 1 | 1 |
| ‘CEREB2--Cerebroside 2’ | 1 | 1 |
| ‘HISOLP--L-Histidinol phosphate’ | 1 | 1 |
| ‘GLNe--L-Glutamine’ | 1 | 2 |
| ‘TYRe--L-Tyrosine’ | 1 | 2 |
| ‘XYLe--D-Xylose’ | 1 | 1 |
| ‘MLT--Maltose’ | 1 | 1 |
| ‘SEDAGP--1,2-diacyl-sn-glycerol 3-phosphate (for Sterolesters)’ | 1 | 1 |
| ‘DMPP--Dimethylallyl diphosphate’ | 1 | 2 |
| ‘LEUe--L-Leucine’ | 1 | 2 |
| ‘FKYN--L-Formylkynurenine’ | 1 | 1 |
| ‘DAGLYP--Diacylglycerol-3-Phosphate’ | 1 | 1 |
| ‘NAGLUSm--N-Acetyl-L-glutamate 5-semialdehyde’ | 1 | 1 |
| ‘GAR--5″-Phosphoribosylglycinamide’ | 1 | 2 |
| ‘EPST--Episterol’ | 1 | 2 |
| ‘E4P--D-Erythrose 4-phosphate’ | 3 | 4 |
| ‘DGDMIPC--Digalactosyl-dimannosyl-inositol-P-ceramide’ | 1 | 1 |
| ‘PHAC--Phenylacetate’ | 2 | 4 |
| ‘UDPG--UDP-glucose’ | 4 | 12 |
| ‘PHEe--L-Phenylalanine’ | 1 | 2 |
| ‘ILEe--L-Isoleucine’ | 1 | 2 |
| ‘C160COA--Hexadecanoyl-CoA’ | 3 | 3 |
| ‘DHMVAm--(R)-2,3-dihydroxy-3-methylbutanoate’ | 1 | 1 |
| ‘CHIT--Chitin’ | 1 | 3 |
| ‘F26P--D-Fructose 2,6-bisphosphate’ | 1 | 1 |
| ‘GLCN--D-Glucosamine’ | 1 | 1 |
| ‘EU1P--L-Erythrulose 1-phosphate’ | 1 | 1 |
| ‘ERGOD--Ergosta-5,7-dienol’ | 1 | 1 |
| ‘XULe--D-Xylulose’ | 1 | 1 |
| ‘G6P--alpha-D-Glucose 6-phosphate’ | 5 | 6 |
| ‘C20SPH--Sphinganine(C20)’ | 1 | 3 |
| ‘TRP--L-Tryptophan’ | 2 | 2 |
| A. thaliana | in | out |
| AraCORE | degree | degree |
| ‘O2[h]--O2, oxygen’ | 3 | 3 |
| ‘NADP[h]--Nicotinamide adenine dinucleotide phosphate’ | 13 | 7 |
| ‘ATP[h]--Adenosine triphosphate’ | 5 | 30 |
| ‘RuBP[h]--Ribulose 1,5-bisphosphate’ | 1 | 2 |
| ‘E4P[h]--Erythrose 4-phosphate’ | 2 | 4 |
| ‘SBP[h]--Sedoheptulose 1,7-bisphosphate’ | 1 | 1 |
| ‘Starch3[h]--Starch, X + 3 glucose units’ | 3 | 3 |
| ‘Starch5[h]--Starch, X + 5 glucose units’ | 1 | 1 |
| ‘Starch1[c]--Starch, X + 1 glucose units’ | 1 | 1 |
| ‘[ATP[c]--Adenosine triphosphate’ | 4 | 31 |
| ‘UDPG[c]--UDP-Glucose’ | 2 | 7 |
| ‘S6P[c]--Sucrose-6-phosphate’ | 1 | 1 |
| ‘T6P[c]--Trehalose 6-phosphate’ | 1 | 1 |
| ‘cellulose3[c]--Cellulose, 3 repeat units’ | 1 | 1 |
| ‘A-CoA[m]--Acetyl-Coenzyme A’ | 1 | 2 |
| ‘ATP[m]--Adenosine triphosphate’ | 2 | 5 |
| ‘PGCA[h]--2-Phosphoglycolate’ | 1 | 1 |
| ‘GCA[h]--Glycolate’ | 1 | 2 |
| ‘GCA[p]--Glycolate’ | 1 | 2 |
| ‘O2[p]--O2, oxygen’ | 2 | 2 |
| ‘GLX[p]--Glyoxylate’ | 1 | 2 |
| ‘NH4[m]--Ammonia’ | 5 | 4 |
| ‘GCEA[p]--Glycerate’ | 2 | 1 |
| ‘GLP[h]--Glucono-delta-lactone-6-phosphate’ | 1 | 1 |
| ‘DAHP[h]--3-Deoxy-D-arabino-heptulosonate-7-phosphate’ | 1 | 1 |
| ‘DHQ[h]--3-Dehydroquinate’ | 1 | 1 |
| ‘SA[h]--Shikimate’ | 1 | 2 |
| ‘EPSP[h]--5-Enolpyruvyl-shikimate-3-phosphate’ | 1 | 1 |
| ‘CHR[h]--Chorismate’ | 1 | 2 |
| ‘PRPP[h]--5-Phosphoribosyl 1-pyrophosphate’ | 1 | 4 |
| ‘A-CoA[c]--Acetyl-Coenzyme A’ | 1 | 3 |
| ‘A-CoA[h]--Acetyl-Coenzyme A’ | 1 | 4 |
| ‘Cit[h]--Citrate’ | 1 | 1 |
| ‘cACN[h]--cis-Aconitate’ | 1 | 1 |
| ‘iCit[c]--Isocitrate’ | 5 | 7 |
| ‘ADN[c]--Adenosine’ | 1 | 3 |
| ‘Rib[c]--Ribose’ | 1 | 1 |
| ‘Rib[h]--Ribose’ | 1 | 1 |
| ‘NO2[c]--Nitrite’ | 2 | 2 |
| ‘SO4[h]--Sulfate’ | 1 | 2 |
| ‘Glu[h]--Glutamate’ | 16 | 17 |
| ‘A-Glu[h]--Acetylglutamate’ | 1 | 1 |
| ‘A-Glu-SeA[h]--Acetylglutamate 5-semialdehyde’ | 1 | 1 |
| ‘A-Orn[h]--Acetylornithine’ | 1 | 1 |
| ‘Gln[h]--Glutamine’ | 2 | 9 |
| ‘CBP[h]--Carbamoyl phosphate’ | 2 | 2 |
| ‘CTL[h]--Citrulline’ | 1 | 2 |
| ‘Asp[h]--Aspartate’ | 1 | 7 |
| ‘Arg-SCA[h]--Argininosuccinate’ | 1 | 1 |
| ‘Arg[h]--Arginine’ | 1 | 2 |
| ‘Asn[c]--Asparagine’ | 2 | 6 |
| ‘A-Ser[c]--O-Acetyl-L-serine’ | 1 | 1 |
| ‘A-Ser[h]--O-Acetyl-L-serine’ | 1 | 1 |
| ‘A-Ser[m]--O-Acetyl-L-serine’ | 1 | 1 |
| ‘Cys[c]--Cysteine’ | 2 | 3 |
| ‘Cys[m]--Cysteine’ | 1 | 1 |
| ‘Gln[m]--Glutamine’ | 1 | 1 |
| ‘Thr[c]--Threonine’ | 2 | 5 |
| ‘GLX[h]--Glyoxylate’ | 1 | 2 |
| ‘PR-ATP[h]--5-Phosphoribosyl-ATP’ | 1 | 1 |
| ‘PR-AMP[h]--5-Phosphoribosyl-AMP’ | 1 | 1 |
| ‘P-AICAR-P[h]--1-(5-Phosphoribosyl)-5-[(5- | 1 | 1 |
| phosphoribosylamino)methylideneamino]imidazole-4-carboxamide’ | | |
| ‘Pu-AICAR-P[h]--5-[(5-phospho-1-deoxyribulos-1-ylamino)methylideneamino]-1- | 1 | 1 |
| (5-phosphoribosyl)imidazole-4-carboxamide’ | | |
| ‘EIGP[h]--Erythro-1-imidazol-4-glycerol 3-phosphate’ | 1 | 1 |
| ‘IA-P[h]--Imidazole acetol-phosphate’ | 1 | 1 |
| ‘Hisol-P[h]--Histidinol phosphate’ | 1 | 1 |
| ‘His[h]--Histidine’ | 1 | 2 |
| ‘Thr[h]--Threonine’ | 1 | 3 |
| ‘DHMP[h]--2,3-Dihydroxy-3-methylpentanoate’ | 1 | 1 |
| ‘DHMB[h]--2,3-Dihydroxy-3-methylbutanoate’ | 1 | 1 |
| ‘2IPM[h]--2-Isopropylmalate’ | 1 | 1 |
| ‘IPM[h]--2-Isopropylmaleate’ | 1 | 1 |
| ‘DAP[h]--Diaminopimelate’ | 1 | 1 |
| ‘Lys[h]--Lysine’ | 1 | 2 |
| ‘PH-Ser[h]--O-Phosphohomoserine’ | 1 | 2 |
| ‘CTH[h]--Cystathionine’ | 1 | 1 |
| ‘H-Cys[h]--Homocysteine’ | 1 | 1 |
| ‘Met[c]--Methionine’ | 3 | 6 |
| ‘Phe[h]--Phenylalanine’ | 1 | 2 |
| ‘Glu-SeA[c]--Glutamate 5-semialdehyde’ | 1 | 1 |
| ‘Glu-SeA[h]--Glutamate 5-semialdehyde’ | 1 | 1 |
| ‘Pro[c]--Proline’ | 2 | 6 |
| ‘P-HPR[h]--3-Phosphohydroxypyruvate’ | 1 | 2 |
| ‘PSer[h]--O-Phosphoserine’ | 1 | 1 |
| ‘PR-ANT[h]--5-Phosphoribosyl-anthranilate’ | 1 | 1 |
| ‘Ind-GP[h]--Indole-glycerol 3-phosphate’ | 1 | 1 |
| ‘Ind[h]--Indole’ | 1 | 1 |
| ‘Trp[h]--Tryptophan’ | 1 | 2 |
| ‘Tyr[h]--Tyrosine’ | 1 | 2 |
| ‘FGAM[h]--5-Phospho-D-ribosyl-N-formylglycineamidine’ | 1 | 1 |
| ‘AIR[h]--5-Aminoimidazole ribonucleotide’ | 1 | 1 |
| ‘CAIR[h]--4-Carboxyaminoimidazole ribonucleotide’ | 1 | 1 |
| ‘FAICAR[h]--5-phosphoribosyl-5-formamido-4-imidazole carboxamide’ | 1 | 1 |
| ‘DC-AMP[h]--N6-(1,2-Dicarboxyethyl)-AMP’ | 1 | 1 |
| ‘GDP[h]--Guanosine diphosphate’ | 1 | 1 |
| ‘CB-Asp[h]--N-Carbamoyl-L-aspartate’ | 1 | 1 |
| ‘DHO[h]--Dihydroorotate’ | 1 | 1 |
| ‘ORO[m]--Orotate’ | 1 | 1 |
| ‘OMP[c]--Orotidine 5-phosphate’ | 1 | 1 |
| ‘CTP[c]--Cytidine triphosphate’ | 1 | 2 |
| ‘dUTP[c]--2-Deoxyuridine 5-triphosphate’ | 1 | 1 |
| ‘GCEA[c]--Glycerate’ | 1 | 2 |
| ‘Arg[c]--Arginine’ | 3 | 6 |
| ‘Lys[c]--Lysine’ | 4 | 4 |
| ‘His[c]--Histidine’ | 3 | 4 |
| ‘DHO[c]--Dihydroorotate’ | 1 | 1 |
| ‘Arg[p]--Arginine’ | 1 | 2 |
| ‘Cys[p]--Cysteine’ | 1 | 2 |
| ‘His[p]--Histidine’ | 1 | 2 |
| ‘Ile[c]--Isoleucine’ | 2 | 4 |
| ‘Ile[p]--Isoleucine’ | 1 | 2 |
| ‘Leu[c]--Leucine’ | 2 | 4 |
| ‘Leu[p]--Leucine’ | 1 | 2 |
| ‘Lys[p]--Lysine’ | 1 | 2 |
| ‘Met[p]--Methionine’ | 1 | 2 |
| ‘Phe[c]--Phenylalanine’ | 2 | 4 |
| ‘Phe[p]--Phenylalanine’ | 1 | 2 |
| ‘Pro[p]--Proline’ | 1 | 2 |
| ‘Thr[p]--Threonine’ | 1 | 2 |
| ‘Trp[c]--Tryptophan’ | 2 | 4 |
| ‘Trp[p]--Tryptophan’ | 1 | 2 |
| ‘Tyr[c]--Tyrosine’ | 2 | 4 |
| ‘Tyr[p]--Tyrosine’ | 1 | 2 |
| ‘Val[c]--Valine’ | 2 | 4 |
| ‘Val[p]--Valine’ | 1 | 2 |
| ‘Asn[h]--Asparagine’ | 1 | 1 |
| ‘Asn[m]--Asparagine’ | 1 | 1 |
| ‘Ile[m]--Isoleucine’ | 1 | 1 |
| ‘Leu[m]--Leucine’ | 1 | 1 |
| ‘Met[h]--Methionine’ | 1 | 1 |
| ‘Met[m]--Methionine’ | 1 | 1 |
| ‘Phe[m]--Phenylalanine’ | 1 | 1 |
| ‘Pro[h]--Proline’ | 1 | 1 |
| ‘Thr[m]--Threonine’ | 1 | 1 |
| ‘Trp[m]--Tryptophan’ | 1 | 1 |
| ‘Tyr[m]--Tyrosine’ | 1 | 1 |
| ‘Val[m]--Valine’ | 1 | 1 |
| C. reinhardtii | in | out |
| iRC1080 | degree | degree |
| 6pgc[h]--6-Phospho-D-gluconate | 1 | 1 |
| 6pgl[h]--6-phospho-D-glucono-1,5-lactone | 1 | 1 |
| atp[h]--ATP | 4 | 7 |
| dhap[h]--Dihydroxyacetone phosphate | 2 | 3 |
| e4p[h]--D-Erythrose 4-phosphate | 3 | 4 |
| fdxox[h]--ferrodoxin (oxidized form 4:2) | 1 | 1 |
| g6p[h]--D-Glucose 6-phosphate | 3 | 4 |
| h2[c]--H2 | 1 | 2 |
| h2[h]--H2 | 2 | 1 |
| mal_L[g]--L-Malate | 1 | 2 |
| nadp[h]--Nicotinamide adenine dinucleotide phosphate | 5 | 4 |
| ppi[h]--Diphosphate | 1 | 1 |
| s17bp[h]--Sedoheptulose 1,7-bisphosphate | 1 | 1 |
| E. coli K12 | in | out |
| iJO1366 | degree | degree |
| ‘14dhncoa[c]--1,4-dihydroxy-2-napthoyl-CoA’ | 1 | 1 |
| ‘14glucan[c]--1,4-alpha-D-glucan’ | 1 | 1 |
| ‘15dap[c]--1,5-Diaminopentane’ | 1 | 1 |
| ‘23dappa[c]--2,3-diaminopropionate’ | 1 | 1 |
| ‘2dda7p[c]--2-Dehydro-3-deoxy-D-arabino-heptonate 7-phosphate’ | 1 | 1 |
| ‘2ddecg3p[c]--2-dodecanoyl-sn-glycerol 3-phosphate’ | 1 | 1 |
| ‘2dh3dgal[c]--2-Dehydro-3-deoxy-D-galactonate’ | 1 | 1 |
| ‘2dr1p[c]--2-Deoxy-D-ribose 1-phosphate’ | 6 | 6 |
| ‘2dr5p[c]--2-Deoxy-D-ribose 5-phosphate’ | 1 | 2 |
| ‘2hdec9eg3p[c]--2-hexadec-9-enoyl-sn-glycerol 3-phosphate’ | 1 | 1 |
| ‘2hdecg3p[c]--2-hexadecanoyl-sn-glycerol 3-phosphate’ | 1 | 1 |
| ‘2mcit[c]--2-Methylcitrate’ | 1 | 1 |
| ‘20dec11eg3p[c]--2-octadec-11-enoyl-sn-glycerol 3-phosphate’ | 1 | 1 |
| ‘2odecg3p[c]--2-octadecanoyl-sn-glycerol 3-phosphate’ | 1 | 1 |
| ‘2p4c2me[c]--2-phospho-4-(cytidine 5″-diphospho)-2-C-methyl-D-erythritol’ | 1 | 1 |
| ‘2sephchc[c]--2-succinyl-5-enolpyruvyl-6-hydroxy-3-cyclohexene-1-carboxylate’ | 1 | 1 |
| ‘2shchc[c]--2-Succinyl-6-hydroxy-2,4-cyclohexadiene-1-carboxylate’ | 1 | 1 |
| ‘2tdec7eg3p[c]--2-tetradec-7-enoyl-sn-glycerol 3-phosphate’ | 1 | 1 |
| ‘2tdecg3p[c]--2-tetradecanoyl-sn-glycerol 3-phosphate’ | 1 | 1 |
| ‘35cgmp[c]--3″,5″-Cyclic GMP’ | 1 | 1 |
| ‘3hcddec5eACP[c]--(R)-3-hydroxy-cis-dodec-5-enoyl-[acyl-carrier protein]’ | 1 | 1 |
| ‘3hcmrs7eACP[c]--(R)-3-hydroxy-cis-myristol-7-eoyl-[acyl-carrier protein]’ | 1 | 1 |
| ‘3hcpalm9eACP[c]--(R)-3-hydroxy-cis-palm-9-eoyl-[acyl-carrier protein]’ | 1 | 1 |
| ‘3hcvac11eACP[c]--(R)-3-hydroxy-cis-vacc-11-enoyl-[acyl-carrier protein]’ | 1 | 1 |
| ‘3php[c]--3-Phosphohydroxypyruvate’ | 1 | 1 |
| ‘4abutn[c]--4-Aminobutanal’ | 1 | 1 |
| ‘4adcho[c]--4-amino-4-deoxychorismate’ | 1 | 1 |
| ‘4c2me[c]--4-(cytidine 5″-diphospho)-2-C-methyl-D-erythritol’ | 1 | 1 |
| ‘4crsol[c]--p-Cresol’ | 1 | 1 |
| ‘4h2opntn[c]--4-Hydroxy-2-oxopentanoate’ | 1 | 1 |
| ‘4mop[c]--4-Methyl-2-oxopentanoate’ | 1 | 1 |
| ‘5aop[c]--5-Amino-4-oxopentanoate’ | 1 | 2 |
| ‘5aprbu[c]--5-Amino-6-(5″-phosphoribitylamino)uracil’ | 1 | 1 |
| ‘5drib[c]--5″-deoxyribose’ | 1 | 1 |
| ‘5mta[c]--5-Methylthioadenosine’ | 1 | 1 |
| ‘6hmhpt[c]--6-hydroxymethyl dihydropterin’ | 1 | 2 |
| ‘aacald[c]--Aminoacetaldehyde’ | 1 | 1 |
| ‘accoa[c]--Acetyl-CoA’ | 19 | 26 |
| ‘acglu[c]--N-Acetyl-L-glutamate’ | 1 | 1 |
| ‘acmana[c]--N-Acetyl-D-mannosamine’ | 1 | 1 |
| ‘acnam[c]--N-Acetylneuraminate’ | 1 | 1 |
| ‘adpgic[c]--ADPglucose’ | 1 | 1 |
| ‘adphep-DD[c]--ADP-D-glycero-D-manno-heptose’ | 1 | 1 |
| ‘adprib[c]--ADPribose’ | 1 | 1 |
| ‘ahcys[c]--S-Adenosyl-L-homocysteine’ | 7 | 1 |
| ‘ahdt[c]--2-Amino-4-hydroxy-6-(erythro-1,2,3-trihydroxypropyl)dihydropteridine | 1 | 1 |
| triphosphate’ | | |
| ‘all-D[c]--D-Allose’ | 1 | 1 |
| ‘alltn[c]--Allantoin’ | 2 | 2 |
| ‘anhgm4p[c]--N-Acetyl-D-glucosamine(anhydrous)N-Acetylmuramyl-tetrapeptide’ | 1 | 3 |
| ‘anhm4p[c]--1,6-anhydrous-N-Acetylmuramyl-tetrapeptide’ | 1 | 2 |
| ‘ap4a[c]--P1,P4-Bis(5″-adenosyl) tetraphosphate’ | 1 | 1 |
| ‘aps[c]--Adenosine 5″-phosphosulfate’ | 1 | 1 |
| ‘ara5p[c]--D-Arabinose 5-phosphate’ | 1 | 2 |
| ‘arbin[c]--aerobactin minus Fe3’ | 3 | 1 |
| ‘arg-L[c]--L-Arginine’ | 4 | 7 |
| ‘ascb6p[c]--L-ascorbate-6-phosphate’ | 1 | 1 |
| ‘atp[c]--ATP’ | 33 | 312 |
| ‘btal[c]--Butanal’ | 1 | 1 |
| ‘camp[c]--CAMP’ | 1 | 1 |
| ‘cddec5eACP[c]--cis-dodec-5-enoyl-[acyl-carrier protein] (n-C12:1)’ | 2 | 1 |
| ‘cdec3eACP[c]--cis-dec-3-enoyl-[acyl-carrier protein] (n-C10:1)’ | 1 | 2 |
| ‘cdpdddecg[c]--CDP-1,2-didodecanoylglycerol’ | 1 | 3 |
| cdpdhdec9eg[c]--CDP-1,2-dihexadec-9-enoylglycerol’ | 1 | 3 |
| ‘cdpdhdecg[c]--CDP-1,2-dihexadecanoylglycerol’ | 1 | 3 |
| cdpdodec11eg[c]--CDP-1,2-dioctadec-11-enoylglycerol’ | 1 | 3 |
| ‘cdpdodecg[c]--CDP-1,2-dioctadecanoylglycerol’ | 1 | 3 |
| cdpdtdec7eg[c]--CDP-1,2-ditetradec-7-enoylglycerol’ | 1 | 3 |
| ‘cdpdtdecg[c]--CDP-1,2-ditetradecanoylglycerol’ | 1 | 3 |
| ‘cgly[c]--Cys-Gly’ | 1 | 1 |
| ‘chor[c]--chorismate’ | 2 | 7 |
| ‘chtbs6p[c]--diacetylchitobiose-6-phosphate’ | 1 | 1 |
| ‘colipa[c]--core oligosaccharide lipid A’ | 1 | 1 |
| ‘cpgn-un[c]--coprogen unloaded (no Fe(III))’ | 3 | 1 |
| ‘cys-D[c]--D-Cysteine’ | 1 | 1 |
| ‘cys-L[c]--L-Cysteine’ | 3 | 9 |
| ‘cyst-L[c]--L-Cystathionine’ | 1 | 1 |
| ‘dad-5[c]--5″-Deoxyadenosine’ | 4 | 1 |
| ‘dhcinnm[c]--2,3-dihydroxicinnamic acid’ | 1 | 1 |
| ‘dhpmp[c]--Dihydroneopterin monophosphate’ | 1 | 1 |
| ‘dhpppn[c]--3-(2,3-Dihydroxyphenyl)propanoate’ | 2 | 1 |
| ‘dhpt[c]--Dihydropteroate’ | 1 | 1 |
| ‘dhptd[c]--4,5-dihydroxy-2,3-pentanedione’ | 1 | 1 |
| ‘dimp[c]--dIMP’ | 1 | 1 |
| ‘ditp[c]--dITP’ | 1 | 1 |
| ‘dmpp[c]--Dimethylallyl diphosphate’ | 2 | 2 |
| ‘dpcoa[c]--Dephospho-CoA’ | 1 | 1 |
| ‘dtdp4d6dg[c]--dTDP-4-dehydro-6-deoxy-D-glucose’ | 1 | 2 |
| ‘dtdpglu[c]--dTDPglucose’ | 1 | 1 |
| ‘e4p[c]--D-Erythrose 4-phosphate’ | 4 | 5 |
| ‘eig3p[c]--D-erythro-1-(Imidazol-4-yl)glycerol 3-phosphate’ | 1 | 1 |
| ‘enter[c]--Enterochelin’ | 4 | 3 |
| ‘f1p[c]--D-Fructose 1-phosphate’ | 1 | 1 |
| ‘fe3dhbzs[c]--ferric 2,3-dihydroxybenzoylserine’ | 1 | 1 |
| ‘fe3hox-un[c]--Fe(III)hydoxamate, unloaded’ | 3 | 1 |
| ‘fecrm-un[c]--Ferrichrome minus Fe(III)’ | 3 | 1 |
| ‘feoxam-un[c]--ferroxamine minus Fe(3)’ | 3 | 1 |
| ‘fpram[c]--2-(Formamido)-N1-(5-phospho-D-ribosyl)acetamidine’ | 1 | 1 |
| ‘g3ps[c]--Glycerophosphoserine’ | 1 | 1 |
| ‘gagicolipa[c]--galactosyl-glucosyl-inner core oligosaccharide lipid A’ | 1 | 1 |
| ‘galctn-D[c]--D-Galactonate’ | 1 | 1 |
| ‘gdpmann[c]--GDP-D-mannose’ | 1 | 2 |
| ‘gdptp[c]--Guanosine 3″-diphosphate 5″-triphosphate’ | 1 | 2 |
| ‘ggagicolipa[c]--glucosyl-galactosyl-glucosyl-inner core oligosaccharide lipid A’ | 1 | 1 |
| gicolipa[c]--glucosyl-inner core oligosaccharide lipid A’ | 1 | 1 |
| ‘gln-L[c]--L-Glutamine’ | 2 | 13 |
| ‘glu-L[c]--L-Glutamate’ | 35 | 27 |
| ‘glyc2p[c]--Glycerol 2-phosphate’ | 1 | 1 |
| ‘gmeACP[c]--Glutaryl-[acyl-carrier protein] methyl ester’ | 1 | 1 |
| gmhep17bp[c]--D-Glycero-D-manno-heptose 1,7-bisphosphate’ | 1 | 1 |
| gmhep7p[c]--D-Glycero-D-manno-heptose 7-phosphate’ | 1 | 1 |
| grdp[c]--Geranyl diphosphate’ | 1 | 1 |
| ‘gtp[c]--GTP’ | 3 | 20 |
| ‘gtspmd[c]--Glutathionylspermidine’ | 1 | 1 |
| ‘h[c]--H+’ | 725 | 341 |
| ‘h2o[c]--H2O’ | 115 | 443 |
| ‘hdeACP[c]--cis-hexadec-9-enoyl-[acyl-carrier protein] (n-C16:1)’ | 3 | 6 |
| ‘hgmeACP[c]--3-Hydroxyglutaryl-[acyl-carrier protein] methyl ester’ | 1 | 1 |
| ‘hhlipa[c]--heptosyl-heptosyl-kdo2-lipidA’ | 1 | 1 |
| ‘hisp[c]--L-Histidinol phosphate’ | 1 | 1 |
| ‘hkndd[c]--2-Hydroxy-6-oxonona-2,4-diene-1,9-dioate’ | 1 | 1 |
| ‘hkntd[c]--2-hydroxy-6-ketononatrienedioate’ | 1 | 1 |
| ‘hmbil[c]--Hydroxymethylbilane’ | 1 | 1 |
| ‘hom-L[c]--L-Homoserine’ | 1 | 4 |
| ‘hphhlipa[c]--heptosyl-phospho-heptosyl-heptosyl-kdo2-lipidA’ | 1 | 1 |
| ‘hpmeACP[c]--3-Hydroxypimeloyl-[acyl-carrier protein] methyl ester’ | 1 | 1 |
| ‘iasp[c]--Iminoaspartate’ | 4 | 1 |
| ‘ichor[c]--Isochorismate’ | 2 | 3 |
| ‘icolipa[c]--inner core oligosaccharide lipid A (E. coli)’ | 1 | 1 |
| ‘imacp[c]--3-(Imidazol-4-yl)-2-oxopropyl phosphate’ | 1 | 1 |
| ‘ipdp[c]--Isopentenyl diphosphate’ | 2 | 5 |
| ‘kdo2lipid4[c]--KDO(2)-lipid IV(A)’ | 1 | 3 |
| ‘kdo8p[c]--3-Deoxy-D-manno-octulosonate 8-phosphate’ | 1 | 1 |
| ‘lgt-S[c]--(R)-S-Lactoylglutathione’ | 1 | 1 |
| ‘lipa_cold[c]--cold adapted KDO(2)-lipid (A)’ | 1 | 1 |
| ‘lipidAds[c]--Lipid A Disaccharide’ | 1 | 1 |
| ‘lipoamp[c]--lipoyl-AMP’ | 1 | 1 |
| ‘lipoate[c]--Lipoate’ | 1 | 1 |
| ‘lyx-L[c]--L-Lyxose’ | 1 | 1 |
| ‘malcoame[c]--malonyl-CoA methyl ester’ | 1 | 1 |
| ‘man6pglyc[c]--2(alpha-D-Mannosyl-6-phosphate)-D-glycerate’ | 1 | 1 |
| ‘mdhdhf[c]--(2R,4S)-2-methyl-2,4-dihydroxydihydrofuran-3-one’ | 1 | 1 |
| ‘methf[c]--5,10-Methenyltetrahydrofolate’ | 3 | 3 |
| ‘mththf[c]--(2R,4S)-2-methyl-2,3,3,4-tetrahydroxytetrahydrofuran’ | 1 | 1 |
| ‘nad[c]--Nicotinamide adenine dinucleotide’ | 71 | 71 |
| ‘op4en[c]--2-Oxopent-4-enoate’ | 2 | 1 |
| ‘oxam[c]--Oxamate’ | 1 | 1 |
| ‘pe120[c]--phosphatidylethanolamine (didodecanoyl, n-C12:0)’ | 2 | 1 |
| ‘pe140[c]--phosphatidylethanolamine (ditetradecanoyl, n-C14:0)’ | 2 | 1 |
| ‘pe141[c]--phosphatidylethanolamine (ditetradec-7-enoyl, n-C14:1)’ | 2 | 1 |
| ‘pe180[c]--phosphatidylethanolamine (dioctadecanoyl, n-C18:0)’ | 2 | 1 |
| ‘pep[c]--Phosphoenolpyruvate’ | 4 | 24 |
| ‘pgp120[c]--Phosphatidylglycerophosphate (didodecanoyl, n-C12:0)’ | 1 | 2 |
| ‘pgp 140[c]--Phosphatidylglycerophosphate (ditetradecanoyl, n-C14:0)’ | 1 | 2 |
| ‘pgp141[c]--Phosphatidylglycerophosphate (ditetradec-7-enoyl, n-C14:1)’ | 1 | 2 |
| ‘pgp 160[c]--Phosphatidylglycerophosphate (dihexadecanoyl, n-C16:0)’ | 1 | 2 |
| ‘pgp161[c]--Phosphatidylglycerophosphate (dihexadec-9-enoyl, n-C16:1)’ | 1 | 2 |
| ‘pgp 180[c]--Phosphatidylglycerophosphate (dioctadecanoyl, n-C18:0)’ | 1 | 2 |
| ‘pgp181[c]--Phosphatidylglycerophosphate (dioctadec-11-enoyl, n-C18:1)’ | 1 | 2 |
| ‘pheme[c]--Protoheme’ | 1 | 3 |
| ‘phom[c]--O-Phospho-L-homoserine’ | 1 | 1 |
| ‘pimACP[c]--Pimeloyl-[acyl-carrier protein]’ | 1 | 1 |
| ‘pmeACP[c]--Pimeloyl-[acyl-carrier protein] methyl ester’ | 1 | 1 |
| ‘pnto-R[c]--(R)-Pantothenate’ | 2 | 1 |
| ‘pran[c]--N-(5-Phospho-D-ribosyl)anthranilate’ | 1 | 1 |
| ‘prbamp[c]--1-(5-Phosphoribosyl)-AMP’ | 1 | 1 |
| ‘prbatp[c]--1-(5-Phosphoribosyl)-ATP’ | 1 | 1 |
| ‘progly[c]--L-Prolinylglycine’ | 1 | 1 |
| ‘prpp[c]--5-Phospho-alpha-D-ribose 1-diphosphate’ | 3 | 12 |
| ‘pser-L[c]--O-Phospho-L-serine’ | 1 | 1 |
| ‘ptrc[c]--Putrescine’ | 5 | 5 |
| ‘r15bp[c]--D-Ribose 1,5-bisphosphate’ | 1 | 1 |
| ‘r1p[c]--alpha-D-Ribose 1-phosphate’ | 6 | 7 |
| ‘rhcys[c]--S-Ribosyl-L-homocysteine’ | 1 | 1 |
| ‘ru5p-D[c]--D-Ribulose 5-phosphate’ | 4 | 4 |
| ‘s7p[c]--Sedoheptulose 7-phosphate’ | 2 | 4 |
| ‘suc6p[c]--Sucrose 6-phosphate’ | 1 | 1 |
| ‘succoa[c]--Succinyl-CoA’ | 4 | 6 |
| ‘sucglu[c]--N2-Succinyl-L-glutamate’ | 1 | 1 |
| ‘sucgsa[c]--N2-Succinyl-L-glutamate 5-semialdehyde’ | 1 | 1 |
| ‘suchms[c]--O-Succinyl-L-homoserine’ | 1 | 1 |
| ‘tartr-D[c]--D-tartrate’ | 1 | 1 |
| ‘tdeACP[c]--cis-tetradec-7-enoyl-[acyl-carrier protein] (n-C14:1)’ | 3 | 5 |
| ‘thm[c]--Thiamin’ | 1 | 1 |
| ‘tre[c]--Trehalose’ | 1 | 1 |
| ‘tre6p[c]--alpha, alpha″-Trehalose 6-phosphate’ | 2 | 2 |
| ‘trnaglu[c]--tRNA (Glu)’ | 1 | 1 |
| ‘u23ga[c]--UDP-2,3-bis(3-hydroxytetradecanoyl)glucosamine’ | 1 | 2 |
| ‘u3aga[c]--UDP-3-O-(3-hydroxytetradecanoyl)-N-acetylglucosamine’ | 1 | 2 |
| ‘u3hga[c]--UDP-3-O-(3-hydroxytetradecanoyl)-D-glucosamine’ | 1 | 1 |
| ‘uLa4fn[c]--undecaprenyl phosphate-4-amino-4-formyl-L-arabinose’ | 1 | 1 |
| ‘uLa4n[c]--undecaprenyl phosphate-4-amino-4-deoxy-L-arabinose’ | 1 | 1 |
| ‘uaagmda[c]--Undecaprenyl-diphospho-N-acetylmuramoyl-(N-acetylglucosamine)- | 1 | 2 |
| L-ala-D-glu-meso-2,6-diaminopimeloyl-D-ala-D-ala’ | | |
| ‘uagmda[c]--Undecaprenyl-diphospho-N-acetylmuramoyl-L-alanyl-D-glutamyl- | 1 | 1 |
| meso-2,6-diaminopimeloyl-D-alanyl-D-alanine’ | | |
| ‘udcpp[c]--Undecaprenyl phosphate’ | 2 | 3 |
| ‘um4p[c]--UDP-N-acetylmuramoyl-L-alanyl-D-gamma-glutamyl-meso-2,6- | 1 | 1 |
| diaminopimelate-D-alanine’ | | |
| ‘unagamuf[c]--Undecaprenyl-diphospho N-acetylglucosamine-N- | 1 | 1 |
| acetylmannosaminuronate-N-acetamido-4,6-dideoxy-D-galactose’ | | |
| ‘uracp[c]--Ureidoacrylate peracid’ | 1 | 1 |
| ‘xan[c]--Xanthine’ | 6 | 4 |
| ‘xtp[c]--XTP’ | 1 | 1 |
| ‘14glucan[e]--1,4-alpha-D-glucan’ | 1 | 2 |
| ‘acmum[e]--N-Acetylmuramate’ | 1 | 2 |
| ‘adocbl[e]--Adenosylcobalamin’ | 1 | 2 |
| ‘arbtn-fe3[e]--Aerobactin’ | 2 | 2 |
| ‘cbl1[e]--Cob(I)alamin’ | 1 | 2 |
| ‘cpgn[e]--coprogen’ | 2 | 2 |
| ‘ddca[e]--Dodecanoate (n-C12:0)’ | 1 | 2 |
| ‘fe3dcit[e]--Fe(III)dicitrate’ | 1 | 2 |
| ‘fe3dhbzs[e]--ferric 2,3-dihydroxybenzoylserine’ | 1 | 2 |
| ‘fe3hox[e]--Fe(III)hydroxamate’ | 2 | 2 |
| ‘fecrm[e]--Ferrichrome’ | 2 | 2 |
| ‘feenter[e]--Fe-enterobactin’ | 2 | 2 |
| ‘feoxam[e]--ferroxamine’ | 2 | 2 |
| ‘gal-bD[e]--beta D-Galactose’ | 2 | 2 |
| ‘hdcea[e]--Hexadecenoate (n-C16:1)’ | 1 | 2 |
| ‘malt[e]--Maltose’ | 1 | 2 |
| ‘malthx[e]--Maltohexaose’ | 1 | 2 |
| ‘maltpt[e]--Maltopentaose’ | 1 | 2 |
| ‘malttr[e]--Maltotriose’ | 1 | 2 |
| ‘maltttr[e]--Maltotetraose’ | 1 | 2 |
| ‘minohp[e]--myo-Inositol hexakisphosphate’ | 1 | 2 |
| ‘nac[e]--Nicotinate’ | 2 | 2 |
| ‘ocdca[e]--octadecanoate (n-C18:0)’ | 1 | 2 |
| ‘ocdcealel--octadecenoate (n-C18:1)’ | 1 | 2 |
| ‘pydam[e]--Pyridoxamine’ | 2 | 2 |
| ‘pydx[e]--Pyridoxal’ | 2 | 2 |
| ‘pydxn[e]--Pyridoxine’ | 2 | 2 |
| ‘ttdca[e]--tetradecanoate (n-C14:0)’ | 1 | 2 |
| ‘ttdcea[e]--tetradecenoate (n-C14:1)’ | 1 | 2 |
| ‘12dgr120[p]--1,2-Diacyl-sn-glycerol (didodecanoyl, n-C12:0)’ | 1 | 1 |
| ‘12dgr140[p]--1,2-Diacyl-sn-glycerol (ditetradecanoyl, n-C14:0)’ | 1 | 1 |
| ‘12dgr141[p]--1,2-Diacyl-sn-glycerol (ditetradec-7-enoyl, n-C14:1)’ | 1 | 1 |
| ‘12dgr160[p]--1,2-Diacyl-sn-glycerol (dihexadecanoyl, n-C16:0)’ | 1 | 1 |
| ‘12dgr180[p]--1,2-Diacyl-sn-glycerol (dioctadecanoyl, n-C18:0)’ | 1 | 1 |
| ‘14glucan[p]--1,4-alpha-D-glucan’ | 1 | 2 |
| ‘1agpe120[p]--1-Acyl-sn-glycero-3-phosphoethanolamine (n-C12:0)’ | 1 | 1 |
| ‘1agpe140[p]--1-Acyl-sn-glycero-3-phosphoethanolamine (n-C14:0)’ | 1 | 1 |
| ‘1agpe141[p]--1-Acyl-sn-glycero-3-phosphoethanolamine (n-C14:1)’ | 1 | 1 |
| ‘1agpe160[p]--1-Acyl-sn-glycero-3-phosphoethanolamine (n-C16:0)’ | 1 | 1 |
| ‘1agpe161[p]--1-Acyl-sn-glycero-3-phosphoethanolamine (n-C 16:1)’ | 1 | 1 |
| ‘1agpe180[p]--1-Acyl-sn-glycero-3-phosphoethanolamine (n-C18:0)’ | 1 | 1 |
| ‘1agpe181[p]--1-Acyl-sn-glycero-3-phosphoethanolamine (n-C18:1)’ | 1 | 1 |
| ‘1agpg120[p]--1-Acyl-sn-glycero-3-phosphoglycerol (n-C12:0)’ | 1 | 1 |
| ‘1agpg140[p]--1-Acyl-sn-glycero-3-phosphoglycerol (n-C14:0)’ | 1 | 1 |
| ‘1agpg141[p]--1-Acyl-sn-glycero-3-phosphoglycerol (n-C14:1)’ | 1 | 1 |
| ‘1agpg160[p]--1-Acyl-sn-glycero-3-phosphoglycerol (n-C16:0)’ | 1 | 1 |
| ‘1agpg161[p]--1-Acyl-sn-glycero-3-phosphoglycerol (n-C16:1)’ | 1 | 1 |
| ‘1agpg180[p]--1-Acyl-sn-glycero-3-phosphoglycerol (n-C18:0)’ | 1 | 1 |
| ‘1agpg181[p]--1-Acyl-sn-glycero-3-phosphoglycerol (n-C18:1)’ | 1 | 1 |
| ‘1ddecg3p[p]--1-dodecanoyl-sn-glycerol 3-phosphate’ | 1 | 1 |
| ‘1hdec9eg3p[p]--1-hexadec-9-enoyl-sn-glycerol 3-phosphate’ | 1 | 1 |
| ‘1hdecg3p[p]--1-hexadecanoyl-sn-glycerol 3-phosphate’ | 1 | 1 |
| ‘1odec11eg3p[p]--1-octadec-11-enoyl-sn-glycerol 3-phosphate’ | 1 | 1 |
| ‘1odecg3p[p]--1-octadecanoyl-sn-glycerol 3-phosphate’ | 1 | 1 |
| ‘1tdec7eg3p[p]--1-tetradec-7-enoyl-sn-glycerol 3-phosphate’ | 1 | 1 |
| ‘1 tdecg3p[p]--1-tetradecanoyl-sn-glycerol 3-phosphate’ | 1 | 1 |
| ‘23dappa[p]--2,3-diaminopropionate’ | 1 | 2 |
| ‘2agpe120[p]--2-Acyl-sn-glycero-3-phosphoethanolamine (n-C12:0)’ | 1 | 1 |
| ‘2agpe140[p]--2-Acyl-sn-glycero-3-phosphoethanolamine (n-C14:0)’ | 1 | 1 |
| ‘2agpe141[p]--2-Acyl-sn-glycero-3-phosphoethanolamine (n-C14:1)’ | 1 | 1 |
| ‘2agpe160[p]--2-Acyl-sn-glycero-3-phosphoethanolamine (n-C16:0)’ | 1 | 1 |
| ‘2agpe161[p]--2-Acyl-sn-glycero-3-phosphoethanolamine (n-C16:1)’ | 1 | 1 |
| ‘2agpe180[p]--2-Acyl-sn-glycero-3-phosphoethanolamine (n-C18:0)’ | 1 | 1 |
| ‘2agpe181[p]--2-Acyl-sn-glycero-3-phosphoethanolamine (n-C18:1)’ | 1 | 1 |
| ‘2agpg120[p]--2-Acyl-sn-glycero-3-phosphoglycerol (n-C12:0)’ | 1 | 1 |
| ‘2agpg140[p]--2-Acyl-sn-glycero-3-phosphoglycerol (n-C14:0)’ | 1 | 1 |
| ‘2agpg141[p]--2-Acyl-sn-glycero-3-phosphoglycerol (n-C14:1)’ | 1 | 1 |
| ‘2agpg160[p]--2-Acyl-sn-glycero-3-phosphoglycerol (n-C16:0)’ | 1 | 1 |
| ‘2agpg161[p]--2-Acyl-sn-glycero-3-phosphoglycerol (n-C16:1)’ | 1 | 1 |
| ‘2agpg180[p]--2-Acyl-sn-glycero-3-phosphoglycerol (n-C18:0)’ | 1 | 1 |
| ‘2agpg181[p]--2-Acyl-sn-glycero-3-phosphoglycerol (n-C18:1)’ | 1 | 1 |
| ‘2ddecg3p[p]--2-dodecanoyl-sn-glycerol 3-phosphate’ | 1 | 1 |
| ‘2hdec9eg3p[p]--2-hexadec-9-enoyl-sn-glycerol 3-phosphate’ | 1 | 1 |
| ‘2hdecg3p[p]--2-hexadecanoyl-sn-glycerol 3-phosphate’ | 1 | 1 |
| ‘2odec11eg3p[p]--2-octadec-11-enoyl-sn-glycerol 3-phosphate’ | 1 | 1 |
| ‘2odecg3p[p]--2-octadecanoyl-sn-glycerol 3-phosphate’ | 1 | 1 |
| ‘2tdec7eg3p[p]--2-tetradec-7-enoyl-sn-glycerol 3-phosphate’ | 1 | 1 |
| ‘2tdecg3p[p]--2-tetradecanoyl-sn-glycerol 3-phosphate’ | 1 | 1 |
| ‘acnam[p]--N-Acetylneuraminate’ | 1 | 2 |
| ‘acolipa[p]--4-Amino-4-deoxy-L-arabinose modified core oligosaccharide lipid A’ | 1 | 1 |
| ‘adocbl[p]--Adenosylcobalamin’ | 1 | 1 |
| ‘all-D[p]--D-Allose’ | 1 | 2 |
| ‘anhgm4p[p]--N-Acetyl-D-glucosamine(anhydrous)N-Acetylmuramyl-tetrapeptide’ | 3 | 3 |
| ‘arbtn[p]--aerobactin minus Fe3’ | 1 | 1 |
| ‘arbtn-fe3[p]--Aerobactin’ | 1 | 1 |
| ‘butso3[p]--butanesulfonate’ | 1 | 2 |
| ‘cbl1[p]--Cob(I)alamin’ | 1 | 1 |
| ‘cgly[p]--Cys-Gly’ | 2 | 2 |
| ‘colipap[p]--core oligosaccharide lipid A diphosphate’ | 1 | 1 |
| ‘cpgn[p]--coprogen’ | 1 | 1 |
| ‘cpgn-un[p]--coprogen unloaded (no Fe(III))’ | 1 | 1 |
| ‘cynt[p]--Cyanate’ | 1 | 2 |
| ‘cys-D[p]--D-Cysteine’ | 1 | 2 |
| ‘eca4colipa[p]--(enterobacterial common antigen)×4 core oligosaccharide lipid A’ | 1 | 1 |
| ‘enter[p]--Enterochelin’ | 1 | 1 |
| ‘ethso3[p]--ethanesulfonate’ | 1 | 2 |
| ‘fe3dcit[p]--Fe(III)dicitrate’ | 1 | 1 |
| ‘fe3dhbzs[p]--ferric 2,3-dihydroxybenzoylserine’ | 1 | 1 |
| ‘fe3hox[p]--Fe(III)hydroxamate’ | 1 | 1 |
| ‘fe3hox-un[p]--Fe(III)hydoxamate, unloaded’ | 1 | 1 |
| ‘fecrm[p]--Ferrichrome’ | 1 | 1 |
| ‘fecrm-un[p]--Ferrichrome minus Fe(III)’ | 1 | 1 |
| ‘feenter[p]--Fe-enterobactin’ | 1 | 1 |
| ‘feoxam[p]--ferroxamine’ | 1 | 1 |
| ‘feoxam-un[p]--ferroxamine minus Fe(3)’ | 1 | 1 |
| ‘g3ps[p]--Glycerophosphoserine’ | 1 | 3 |
| ‘gal-bD[p]--beta D-Galactose’ | 1 | 2 |
| ‘galctn-D[p]--D-Galactonate’ | 1 | 2 |
| ‘galctn-L[p]--L-Galactonate’ | 1 | 2 |
| ‘glyc2p[p]--Glycerol 2-phosphate’ | 1 | 3 |
| ‘h[p]--H+’ | 161 | 194 |
| ‘isetac[p]--Isethionic acid’ | 1 | 2 |
| ‘kdo2lipid4[p]--KDO(2)-lipid IV(A)’ | 1 | 1 |
| ‘lipa_cold[p]--cold adapted KDO(2)-lipid (A)’ | 1 | 1 |
| ‘lipoate[p]--Lipoate’ | 1 | 2 |
| ‘lyx-L[p]--L-Lyxose’ | 1 | 2 |
| ‘malt[p]--Maltose’ | 1 | 1 |
| ‘maltpt[p]--Maltopentaose’ | 1 | 1 |
| ‘malttr[p]--Maltotriose’ | 1 | 1 |
| ‘maltttr[p]--Maltotetraose’ | 1 | 1 |
| ‘mmet[p]--S-Methyl-L-methionine’ | 1 | 2 |
| ‘mobd[p]--Molybdate’ | 1 | 2 |
| ‘mso3[p]--methanesulfonate’ | 1 | 2 |
| ‘murein3px3p[p]--two disacharide linked murein units, tripeptide crosslinked | 1 | 1 |
| tripeptide (A2pm->A2pm) (middle of chain)’ | | |
| ‘murein4px4p4p[p]--three disacharide linked murein units (tetrapeptide crosslinked | 1 | 1 |
| tetrapeptide (A2pm->D-ala), one uncrosslinked tetrapaptide) (middle of chain)’ | | |
| ‘murein4px4px4p[p]--three disacharide linked murein units (tetrapeptide crosslinked | 1 | 2 |
| tetrapeptide (A2pm->D-ala) & tetrapeptide corsslinked tetrapeptide (A2pm->D-ala)) | | |
| (middle of chain)’ | | |
| ‘murein5p5p[p]--two linked disacharide pentapeptide murein units (uncrosslinked, | 1 | 5 |
| middle of chain)’ | | |
| ‘murein5p5p5p[p]--three linked disacharide pentapeptide murein units | 1 | 1 |
| (uncrosslinked, middle of chain)’ | | |
| ‘murein5px3p[p]--two disacharide linked murein units, pentapeptide corsslinked | 1 | 2 |
| tripeptide (A2pm->A2pm) (middle of chain)’ | | |
| ‘murein5px4p[p]--two disacharide linked murein units, pentapeptide crosslinked | 1 | 3 |
| tetrapeptide (A2pm->D-ala) (middle of chain)’ | | |
| ‘murein5px4px4p[p]--three disacharide linked murein units (pentapeptide | 1 | 1 |
| crosslinked tetrapeptide (A2pm->D-ala) tetrapeptide corsslinked tetrapeptide | | |
| (A2pm->D-ala)) (middle of chain)’ | | |
| ‘nac[p]--Nicotinate’ | 1 | 2 |
| ‘pa120[p]--1,2-didodecanoyl-sn-glycerol 3-phosphate’ | 2 | 3 |
| ‘pa140[p]--1,2-ditetradecanoyl-sn-glycerol 3-phosphate’ | 2 | 3 |
| ‘pa141[p]--1,2-ditetradec-7-enoyl-sn-glycerol 3-phosphate’ | 2 | 3 |
| ‘pa160[p]--1,2-dihexadecanoyl-sn-glycerol 3-phosphate’ | 2 | 3 |
| ‘pa161[p]--1,2-dihexadec-9-enoyl-sn-glycerol 3-phosphate’ | 2 | 3 |
| ‘pa180[p]--1,2-dioctadecanoyl-sn-glycerol 3-phosphate’ | 2 | 3 |
| ‘pa181[p]--1,2-dioctadec-11-enoyl-sn-glycerol 3-phosphate’ | 2 | 3 |
| ‘pe120[p]--phosphatidylethanolamine (didodecanoyl, n-C12:0)’ | 1 | 2 |
| ‘pe140[p]--phosphatidylethanolamine (ditetradecanoyl, n-C14:0)’ | 1 | 2 |
| ‘pe141[p]--phosphatidylethanolamine (ditetradec-7-enoyl, n-C14:1)’ | 1 | 2 |
| ‘pe160[p]--phosphatidylethanolamine (dihexadecanoyl, n-C16:0)’ | 1 | 3 |
| ‘pe161[p]--phosphatidylethanolamine (dihexadec-9enoyl, n-C16:1)’ | 1 | 4 |
| ‘pe180[p]--phosphatidylethanolamine (dioctadecanoyl, n-C18:0)’ | 1 | 2 |
| ‘pe181[p]--phosphatidylethanolamine (dioctadec-11-enoyl, n-C18:1)’ | 1 | 4 |
| ‘pg120[p]--Phosphatidylglycerol (didodecanoyl, n-C12:0)’ | 4 | 3 |
| ‘pg140[p]--Phosphatidylglycerol (ditetradecanoyl, n-C14:0)’ | 4 | 3 |
| ‘pg141[p]--Phosphatidylglycerol (ditetradec-7-enoyl, n-C14:1)’ | 4 | 3 |
| ‘pg160[p]--Phosphatidylglycerol (dihexadecanoyl, n-C16:0)’ | 4 | 4 |
| ‘pg161[p]--Phosphatidylglycerol (dihexadec-9-enoyl, n-C16:1)’ | 4 | 4 |
| ‘pg180[p]--Phosphatidylglycerol (dioctadecanoyl, n-C18:0)’ | 4 | 3 |
| ‘pg181[p]--Phosphatidylglycerol (dioctadec-11-enoyl, n-C18:1)’ | 4 | 4 |
| ‘pgp120[p]--Phosphatidylglycerophosphate (didodecanoyl, n-C12:0)’ | 1 | 1 |
| ‘pgp140[p]--Phosphatidylglycerophosphate (ditetradecanoyl, n-C14:0)’ | 1 | 1 |
| ‘pgp141[p]--Phosphatidylglycerophosphate (ditetradec-7-enoyl, n-C14:1)’ | 1 | 1 |
| ‘pgp 160[p]--Phosphatidylglycerophosphate (dihexadecanoyl, n-C16:0)’ | 1 | 1 |
| ‘pgp161[p]--Phosphatidylglycerophosphate (dihexadec-9-enoyl, n-C16:1)’ | 1 | 1 |
| ‘pgp180[p]--Phosphatidylglycerophosphate (dioctadecanoyl, n-C18:0)’ | 1 | 1 |
| ‘pgp181[p]--Phosphatidylglycerophosphate (dioctadec-11-enoyl, n-C18:1)’ | 1 | 1 |
| ‘pheme[p]--Protoheme’ | 1 | 1 |
| ‘progly[p]--L-Prolinylglycine’ | 1 | 2 |
| ‘pydam[p]--Pyridoxamine’ | 1 | 2 |
| ‘pydx[p]--Pyridoxal’ | 1 | 2 |
| ‘pydxn[p]--Pyridoxine’ | 1 | 2 |
| ‘sulfac[p]--sulfoacetate’ | 1 | 2 |
| ‘taur[p]--Taurine’ | 1 | 2 |
| ‘thm[p]--Thiamin’ | 1 | 2 |
| H. sapiens | in | out |
| recon1 | degree | degree |
| 10fthf6glu_m--10fthf6glu c | 1 | 2 |
| 10fthf7glu_c--10fthf7glu c | 2 | 1 |
| 10fthf7glu_l--10fthf7glu c | 1 | 1 |
| 10fthf7glu_m--10fthf7glu c | 1 | 1 |
| h2o_c--H2O | 100 | 237 |
| bamppald_c--Beta-Aminopropion aldehyde | 1 | 1 |
| o2_c--O2 | 8 | 80 |
| h_c-H+ | 351 | 243 |
| atp_c--ATP | 71 | 190 |
| Lpipecol_x--L-Pipecolate | 1 | 1 |
| 2425dhvitd2_m--2425dhvitd2 c | 1 | 1 |
| 2425dhvitd3_m--2425dhvitd3 c | 1 | 1 |
| 2425dhvitd2_c--2425dhvitd2 c | 1 | 1 |
| 2425dhvitd3_c--2425dhvitd3 c | 1 | 1 |
| paps_c--3’-Phosphoadenylyl sulfate | 1 | 12 |
| nadph_c--Nicotinamide adenine dinucleotide phosphate-reduced | 27 | 77 |
| akg_c--2-Oxoglutarate | 15 | 14 |
| drib_c--Deoxyribose C5H10O4 | 2 | 2 |
| nadh_c--Nicotinamide adenine dinucleotide-reduced | 50 | 43 |
| nad_c--Nicotinamide adenine dinucleotide | 45 | 52 |
| amet_c--S-Adenosyl-L-methionine | 4 | 9 |
| hgentis_c--Homogentisate C8H7O4 | 1 | 1 |
| 34hpp_c--3-(4-Hydroxyphenyl)pyruvate | 1 | 2 |
| 3aib_m--3aib c | 1 | 2 |
| 3aib_c--3aib c | 1 | 1 |
| 3hanthrn_c--3 Hydroxyanthranilate C7H7NO3 | 1 | 1 |
| cmusa_c--2 Amino 3 carboxymuconate semialdehyde C7H6NO5 | 1 | 2 |
| hLkynr_c--3 Hydroxy L kynurenine C10H12N2O4 | 1 | 1 |
| h2o_l--H2O | 2 | 25 |
| 3snpyr_c--3snpyr c | 1 | 1 |
| 3snpyr_m--3snpyr c | 1 | 1 |
| adrnl_c--Adrnl c | 1 | 3 |
| 5aop_c--5-Amino-4-oxopentanoate | 1 | 2 |
| 6dhf_m--6dhf c | 1 | 2 |
| 6thf_m--6thf c | 1 | 2 |
| 7dhf_l--7dhf c | 1 | 1 |
| 7dhf_c--7dhf c | 2 | 1 |
| 7dhf_m--7dhf c | 1 | 1 |
| 7thf_c--7thf c | 2 | 1 |
| 7thf_l--7thf c | 1 | 1 |
| 7thf_m--7thf c | 1 | 1 |
| aact_m--Aminoacetone | 1 | 1 |
| L2aadp6sa_c--L 2 Aminoadipate 6 semialdehyde C6H11NO3 | 1 | 1 |
| ppa_c--Propionate (n-C3:0) | 1 | 3 |
| acmana_c--N-Acetyl-D-mannosamine | 3 | 3 |
| acnamp_c--Acnamp c | 1 | 1 |
| acmanap_c--N-Acetyl-D-mannosamine 6-phosphate | 1 | 1 |
| man6p_c--D-Mannose 6-phosphate | 2 | 2 |
| kdnp_c--Kdnp c | 1 | 1 |
| fad_m--Flavin adenine dinucleotide oxidized | 29 | 33 |
| fadh2_m--Flavin adenine dinucleotide reduced | 33 | 29 |
| ribflv_c--Riboflavin | 1 | 1 |
| fmn_c--FMN | 2 | 2 |
| 3pg_c--3-Phospho-D-glycerate | 4 | 3 |
| adrncoa_c--Adrncoa c | 3 | 4 |
| crn_c--L-Carnitine | 5 | 31 |
| adrnorn_c--Adrnorn c | 1 | 1 |
| adrnorn_m--Adrnorn c | 1 | 1 |
| dcamp_c--N6-(1,2-Dicarboxyethyl)-AMP | 1 | 1 |
| alpa_hs_c--Alpa hs c | 1 | 1 |
| Rtotal2coa_c--Rtotal2coa c | 3 | 5 |
| ala__L_x--L-Alanine | 1 | 1 |
| pyr_x--Pyruvate | 3 | 2 |
| hcys__L_c--L-Homocysteine | 1 | 4 |
| asn__L_c--L-Asparagine | 8 | 7 |
| cys__L_c--L-Cysteine | 13 | 15 |
| ala__D_c--D-Alanine | 4 | 5 |
| gln__L_c--L-Glutamine | 14 | 23 |
| 12ppd__S_c--(S)-Propane-1,2-diol | 1 | 1 |
| arachd_c--Arachd c | 4 | 7 |
| 5HPET_c--5HPET c | 1 | 1 |
| am6sa_c--2-Aminomuconate 6-semialdehyde | 1 | 1 |
| h2o_r-H2O | 23 | 15 |
| cholcoar_x--Cholcoar r | 1 | 1 |
| cgly_c--Cys-Gly | 1 | 1 |
| lys__L_c--L-Lysine | 5 | 6 |
| apoC_c--ApoC c | 1 | 1 |
| apoC_Lys_btn_c--ApoC Lys btn c | 1 | 1 |
| apoC_Lys_btn_m--ApoC Lys btn c | 1 | 1 |
| biocyt_m--Biocyt c | 1 | 1 |
| arachcoa_c--Arachidyl coenzyme A | 2 | 4 |
| arachorn_c--Arachorn c | 1 | 1 |
| arachorn_m--Arachorn c | 1 | 1 |
| arachdcoa_c--Arachdcoa c | 4 | 5 |
| arg__L_c--L-Arginine | 6 | 5 |
| Rtotalcoa_c--Rtotalcoa c | 8 | 6 |
| Rtotal3coa_c--Rtotal3coa c | 3 | 4 |
| pmtcoa_c--Palmitoyl-CoA (n-C16:0CoA) | 6 | 9 |
| hdcoa_c--Hexadecenoyl-CoA (n-C16:1CoA) | 2 | 4 |
| tdcoa_c--Tetradecanoyl-CoA (n-C14:0CoA) | 2 | 3 |
| lnincacoa_c--Lnincacoa c | 1 | 4 |
| lnincgcoa_c--Lnincgcoa c | 2 | 4 |
| strdnccoa_c--Strdnccoa c | 2 | 4 |
| dinlogcoa_c--Dinicgcoa c | 2 | 4 |
| tmndnccoa_c--Tmndnccoa c | 3 | 5 |
| lniccoa c--Lniccoa c | 1 | 4 |
| dcsptn1coa_c--Dosptnicoa c | 2 | 4 |
| c226coa_c--C226coa c | 3 | 4 |
| stcoa_c--Stearoyl-CoA (n-C18:0CoA) | 3 | 6 |
| odecoa_c--Octadecenoyl-CoA (n-C18:1CoA) | 3 | 5 |
| vacccoa_c--Vacccoa c | 2 | 5 |
| lneldccoa_c--Lneldccoa c | 4 | 4 |
| od2coa_c--Trans-Octadec-2-enoyl-CoA | 2 | 4 |
| eicostetcoa_c--Eicostetcoa c | 2 | 4 |
| R6coa_hs_c--R6coa hs c | 1 | 1 |
| hdd2coa_c--Trans-Hexadec-2-enoyl-CoA | 1 | 3 |
| crm_hs_l--Crm hs c | 1 | 1 |
| asn__L_m--L-Asparagine | 1 | 1 |
| Nacasp_m--Nacasp c | 1 | 1 |
| atp_n--ATP | 16 | 27 |
| avite1_e--Avite1 c | 1 | 2 |
| avite1_c--Avite1 c | 1 | 1 |
| avite2_c--Avite2 c | 1 | 1 |
| avite2_e--Avite2 c | 1 | 2 |
| man_l--D-Mannose | 2 | 1 |
| mn_l--Mn c | 1 | 1 |
| dgcholcoa_x--Dgcholcoa x | 1 | 1 |
| 3aib__D_c--3aib D c | 1 | 1 |
| bvite_e--Bvite c | 1 | 2 |
| bvite_c--Bvite c | 1 | 1 |
| pmtcrn_c--Pmtcrn c | 1 | 1 |
| pmtcrn_m--Pmtcrn c | 1 | 1 |
| hdcecrn_c--Hdcecrn c | 1 | 1 |
| hdd2crn_c--Hdd2crn c | 1 | 1 |
| hdcecrn_m--Hdcecrn c | 1 | 1 |
| hdd2crn_m--Hdd2crn c | 1 | 1 |
| stcrn_c--Stcrn c | 1 | 1 |
| stcrn_m--Stcrn c | 1 | 1 |
| odecrn_c--Odecrn c | 1 | 1 |
| odecrn_m--Odecrn c | 1 | 1 |
| arachdcrn_c--Arachdcrn c | 1 | 1 |
| arachdcn_m--Arachdcrn c | 1 | 1 |
| c226crn_c--C226crn c | 1 | 1 |
| c226crn_m--C226crn c | 1 | 1 |
| dag_hs_c--Dag hs c | 8 | 5 |
| chsterols_c--Chsterols c | 1 | 1 |
| pcrn_x--Pcrn c | 1 | 2 |
| pcrn_c--Pcrn c | 1 | 1 |
| tsul_m--Thiosulfate | 2 | 3 |
| tcynt_m--Thiocyanate | 1 | 1 |
| cysam_c--Cysam c | 1 | 1 |
| cyst__L_c--L-Cystathionine | 1 | 1 |
| dcsptn1crn_c--Dcsptn1crn c | 1 | 1 |
| dcsptn1crn_m--Dcsptn1crn c | 1 | 1 |
| 56dihindlcrbxlt_c--56dihindicrbxlt c | 1 | 2 |
| L_dpchrm_c--L dpchrm c | 2 | 1 |
| tag_hs_c--Tag hs c | 1 | 1 |
| fadh2_r--Flavin adenine dinucleotide reduced | 1 | 3 |
| zymstnl_r--Zymstnl r | 1 | 1 |
| dheas_c--Dheas c | 1 | 1 |
| 56dura_c--5,6-dihydrouracil | 1 | 2 |
| 56dthm_c--56dthm c | 1 | 2 |
| dlnlcgern_c--Dlnlcgorn c | 1 | 1 |
| dlnlcgorn_m--Dlnicgern c | 1 | 1 |
| ipdp_x--Isopentenyl diphosphate | 2 | 3 |
| dmpp_x--Dimethylallyl diphosphate | 1 | 2 |
| grdp_x--Geranyl diphosphate | 1 | 1 |
| 13_cis_oretn_n--13 cis oretn c | 1 | 2 |
| 13_cis_retn_n--13 cis retn c | 1 | 2 |
| ethamp_r--Ethanolamine phosphate C2H7NO4P | 1 | 1 |
| hretn_n--Hretn c | 1 | 2 |
| kdn_c--Kdn c | 1 | 1 |
| melanin_c--Melanin c | 1 | 1 |
| oretn_n--Oretn c | 1 | 2 |
| sprm_c--Spermine C10H30N4 | 1 | 1 |
| yvite_c--Yvite c | 1 | 1 |
| dopasf_c--Dopasf c | 1 | 1 |
| pan4p_c--Pantetheine 4’-phosphate | 3 | 2 |
| 5dpmev_x--R 5 Diphosphomevalonate C6H10O10P2 | 1 | 1 |
| 2dr5p_c--2-Deoxy-D-ribose 5-phosphate | 1 | 1 |
| dump_m--DUMP | 3 | 2 |
| eicostetcrn_c--Eicostetcrn c | 1 | 1 |
| eicostetcrn_m--Eicostetcrn c | 1 | 1 |
| elaidcrn_c--Elaidcrn c | 1 | 1 |
| elaidcrn_m--Elaidcrn c | 1 | 1 |
| n2m2nm_l--N2m2nm c | 1 | 1 |
| s2l2n2m2m_l--S2l2n2m2m c | 1 | 1 |
| gluala_e--5 L Glutamyl L alanine C8H13N2O5 | 2 | 2 |
| ha_e--Ha e | 2 | 2 |
| n2m2nmasn_e--N2m2nmasn e | 1 | 2 |
| nac_e--Nicotinate | 1 | 2 |
| orn_e--Ornithine | 1 | 2 |
| ppa_e--Propionate (n-C3:0) | 3 | 2 |
| retpalm_e--All-trans-Retinyl palmitate | 2 | 2 |
| s2l2n2m2masn_e--S2l2n2m2masn e | 2 | 2 |
| sarcs_e--Sarcosine | 1 | 2 |
| tagat__D_e--Tagat D c | 1 | 2 |
| ttdca_e--Tetradecanoate (n-C14:0) | 1 | 2 |
| yvite_e--Yvite c | 1 | 2 |
| ptdcacoa_c--Ptdcacoa c | 1 | 2 |
| hpdcacoa_c--Hpdcacoa c | 1 | 2 |
| f6p_c--D-Fructose 6-phosphate | 8 | 7 |
| f26bp_c--D Fructose 2 6 bisphosphate C6H10O12P2 | 1 | 1 |
| fe2 c--Fe2+ | 1 | 1 |
| Lfmkynr_c--L Formylkynurenine C11H12N2O4 | 1 | 1 |
| Lkynr_c--L Kynurenine C10H12N2O3 | 1 | 1 |
| 5dhf_m--5dhf c | 1 | 1 |
| s2l2n2m2masn_l--S2l2n2m2masn e | 1 | 1 |
| 4fumacac_c--4 Fumarylacetoacetate C8H6O6 | 1 | 1 |
| glyc3p_c--Glycerol 3-phosphate | 3 | 3 |
| 6pgl_c--6-phospho-D-glucono-1,5-lactone | 1 | 2 |
| gal_l--D-Galactose | 1 | 1 |
| n2m2nmasn_l--N2m2nmasn e | 1 | 1 |
| glyclt_c--Glycolate | 2 | 1 |
| glygn1_c--Glygn1 c | 1 | 1 |
| ha_l--Ha e | 1 | 1 |
| ha deg1_\|--Ha deg1 \| | 1 | 1 |
| dxtrn_c--Dxtrn c | 1 | 1 |
| gln__L_m--L-Glutamine | 1 | 1 |
| oxa_x--Oxalate | 1 | 1 |
| glyc__R_c--(R)-Glycerate | 1 | 1 |
| glyclt_x--Glycolate | 1 | 1 |
| glyc__S_c--Glyc S c | 1 | 1 |
| g3pc_c--Sn-Glycero-3-phosphocholine | 1 | 1 |
| hdca_r--Hexadecanoate (n-C16:0) | 1 | 1 |
| glcur1p_c--D-Glucuronate 1-phosphate | 1 | 1 |
| udpglcur_c--UDP-D-glucuronate | 2 | 5 |
| 4mlacac_c--4 Maleylacetoacetate C8H6O6 | 1 | 1 |
| his__L_c--L-Histidine | 3 | 3 |
| urcan_c--Urocanate | 1 | 1 |
| hista_c--Hista c | 2 | 3 |
| hmbil_c--Hydroxymethylbilane | 1 | 1 |
| mev__R_x--R Mevalonate C6H11O4 | 1 | 1 |
| hpdcacrn_c--Hpdcacrn c | 1 | 1 |
| hpdcacrn_m--Hpdcacrn c | 1 | 1 |
| hretn_c--Hretn c | 2 | 1 |
| lnlncacrn_c--Lnlncacrn c | 1 | 1 |
| lnlncacrn_m--Lnlncacrn c | 1 | 1 |
| dhocholoylcoa_x--Dhocholoylcoa x | 1 | 1 |
| cholcoaone_x--Cholcoaone x | 1 | 1 |
| hxan_c--Hypoxanthine | 3 | 5 |
| 4izp_c--4-Imidazolone-5-propanoate | 1 | 1 |
| tagat__D_c--Tagat D c | 1 | 1 |
| lneldccrn_c--Lneldccrn c | 1 | 1 |
| lneldccrn_m--Lneldccrn c | 1 | 1 |
| lnlccrn_c--Lnlccrn c | 1 | 1 |
| lnlccrn_m--Lnlccrn c | 1 | 1 |
| lnlncgcrn_c--Lnlncgcrn c | 1 | 1 |
| lnlncgcrn_m--Lnlncgcrn c | 1 | 1 |
| Ssq23epx_r--S Squalene 2 3 epoxide C30H50O | 1 | 1 |
| lpchol_hs_c--Lpchol hs c | 4 | 1 |
| trp__L_c--L-Tryptophan | 2 | 3 |
| 6a2ohxnt x--6-Amino-2-oxohexanoate | 1 | 1 |
| 3mldz_c--3mldz c | 1 | 1 |
| mercplaccys_c--Mercplaccys c | 1 | 1 |
| 5mdru1p_c--5-Methylthio-5-deoxy-D-ribulose 1-phosphate | 1 | 2 |
| 5pmev_x--R 5 Phosphomevalonate C6H10O7P | 1 | 1 |
| mi134p_c--Mi134p c | 1 | 2 |
| mi1345p_c--1D myo Inositol 1 3 4 5 tetrakisphosphate C6H8O18P4 | 1 | 1 |
| mi13p_c--Mi13p c | 1 | 1 |
| mi34p_c--Mi34p c | 1 | 1 |
| mi145p_c--1D myo Inositol 1 4 5 trisphosphate C6H9O15P3 | 1 | 2 |
| mi14p_c--Mi14p c | 3 | 3 |
| mi4p__D_c--1D-myo-Inositol 4-phosphate | 1 | 1 |
| mi3p__D_c--1D-myo-Inositol 3-phosphate | 1 | 1 |
| 5mdr1p_c--5-Methylthio-5-deoxy-D-ribose 1-phosphate | 2 | 1 |
| Nacasp_c--Nacasp c | 1 | 1 |
| ncam_c--Nicotinamide | 3 | 3 |
| nrpphrsf_c--Nrpphrsf c | 1 | 1 |
| dtmp_m--DTMP | 1 | 1 |
| retn_c--Retn c | 4 | 7 |
| oretn_c--Oretn c | 3 | 2 |
| 13_cis_retn_c--13 cis retn c | 4 | 6 |
| 13_cis_oretn_c--13 cis oretn c | 3 | 2 |
| phaccoa_c--Phenylacetyl-CoA | 1 | 1 |
| paf_hs_c--Paf hs c | 1 | 1 |
| ak2lgchol_hs_c--Ak2lgchol hs c | 1 | 1 |
| pail_hs_n--Pail hs c | 3 | 5 |
| ptth_c--Pantetheine | 1 | 1 |
| pchol_hs_m--Pchol hs c | 1 | 2 |
| 3php_c--3-Phosphohydroxypyruvate | 1 | 1 |
| prostgh2_r--Prostgh2 c | 1 | 1 |
| phe__L_c--L-Phenylalanine | 2 | 3 |
| pail5p_hs_n--Pail5p hs c | 1 | 1 |
| pnto__R_c--(R)-Pantothenate | 2 | 1 |
| 4ppcys_c--N-((R)-4-Phosphopantothenoyl)-L-cysteine | 1 | 1 |
| fpram_c--2-(Formamido)-N1-(5-phospho-D-ribosyl)acetamidine | 1 | 1 |
| prgnlones_c--Prgnlones c | 1 | 1 |
| pser__L_c--O-Phospho-L-serine | 1 | 1 |
| ptdcacrn_c--Ptdcacrn c | 1 | 1 |
| ptdcacrn_m--Ptdcacrn c | 1 | 1 |
| retnglc_c--Retnglc c | 1 | 1 |
| 13_cis_retnglc_c--13 cis retnglc c | 1 | 1 |
| 13_cis_retnglc_r--13 cis retnglc c | 1 | 1 |
| retnglc_r--Retnglc c | 1 | 1 |
| Rtotal2crn_c--Rtotal2crn c | 1 | 1 |
| Rtotal2crn_m--Rtotal2crn c | 1 | 4 |
| Rtotal3crn_c--Rtotal3crn c | 1 | 1 |
| Rtotal3crn_m--Rtotal3crn c | 1 | 1 |
| Rtotalcrn_c--Rtotalcrn c | 1 | 1 |
| Rtotalcrn_m--Rtotalcrn c | 1 | 1 |
| sphs1p_r--Sphs1p c | 1 | 3 |
| sbt__D_c--D-Sorbitol | 1 | 1 |
| sphmyln_hs_l--Sphmyln hs c | 1 | 2 |
| strdnccrn_c--Strdnccrn c | 1 | 1 |
| stranccrn_m--Stranccrn c | 1 | 1 |
| tmndnccrn_c--Tmndnccrn c | 1 | 1 |
| tmndnccrn_m--Tmndnccrn c | 1 | 1 |
| triodthysuf_c--Triodthysuf c | 1 | 1 |
| ttdcrn_c--Ttdcrn c | 1 | 1 |
| ttdcrn_m--Ttdcrn c | 1 | 1 |
| tymsf_c--Tymsf c | 1 | 1 |
| urate_c--Urate | 1 | 1 |
| urate_x--Urate | 2 | 1 |
| vacccrn_c--Vacccrn c | 1 | 1 |
| vacccrn_m--Vacccrn c | 1 | 1 |
| M. acetivorans | in | out |
| iMB745 | degree | degree |
| 3dhq[c]--3dhq[c] | 1 | 2 |
| 3hdggpgp[c]--3hdggpgp[c] | 1 | 1 |
| 4adhq[c]--4adho[c] | 1 | 1 |
| 4as[c]--4as[c] | 1 | 1 |
| 4das[c]--4das[c] | 1 | 1 |
| 5aprbu[c]--5aprbu[c] | 1 | 1 |
| 5pr5hbz[c]--5pr5hbz[c] | 1 | 1 |
| 6hmhptpp[c]--6hmhptpp[c] | 1 | 2 |
| 7mhp[c]--7mhp[c] | 1 | 1 |
| accoa[c]--accoa[c] | 8 | 14 |
| agdpgpi[c]--agdpgpi[c] | 1 | 1 |
| atp[c]--atp[c] | 35 | 114 |
| biomass_met[c]--biomass_met[c] | 1 | 1 |
| cbl1[c]--cbl1[c] | 1 | 1 |
| cbl1[e]--cbl1[e] | 1 | 2 |
| cbl1hbi[e]--cbl1hbil[e] | 1 | 2 |
| ctp[c]--ctp[c] | 4 | 9 |
| cu2[c]--cu2[c] | 1 | 1 |
| cu2[e]--cu2[e] | 1 | 1 |
| dggpgp[c]--dggpgp[c] | 1 | 1 |
| dhadrp[c]--dhadrp[c] | 1 | 1 |
| dhap[c]--dhap[c] | 4 | 5 |
| dhnpt[c]--dhnpt[c] | 1 | 1 |
| dhp23cp[c]--dhp23cp[c] | 1 | 1 |
| dhpmp[c]--dhpmp[c] | 1 | 1 |
| dhpt[c]--dhpt[c] | 1 | 1 |
| dkfp[c]--dkfp[c] | 1 | 2 |
| dohau[c]--dohau[c] | 1 | 1 |
| dohdu[c]--dohdu[c] | 1 | 1 |
| dpgpi[c]--dpgpi[c] | 1 | 2 |
| dscl[c]--dscl[c] | 1 | 3 |
| dtmp[c]--dtmp[c] | 3 | 2 |
| eig3p[c]--eig3p[c] | 1 | 1 |
| f430p3[c]--f430p3[c] | 1 | 1 |
| fapy[c]--fapy[c] | 1 | 1 |
| fmn[c]--fmn[c] | 1 | 1 |
| g6p[c]--g6p[c] | 3 | 3 |
| gar[c]--gar[c] | 1 | 1 |
| gln_L[c]--gln-L[c] | 2 | 15 |
| glu_L[c]--glu-L[c] | 32 | 34 |
| glu5sa[c]--glu5sa[c] | 1 | 1 |
| glycogen[c]--glycogen[c] | 1 | 3 |
| gmp[c]--gmp[c] | 8 | 4 |
| gtp[c]--gtp[c] | 3 | 21 |
| h[c]--h[c] | 258 | 174 |
| hisp[c]--hisp[c] | 1 | 2 |
| iasp[c]--iasp[c] | 2 | 1 |
| imp[c]--imp[c] | 3 | 4 |
| ipdp[c]--ipdp[c] | 2 | 9 |
| lald_L[c]--lald-L[c] | 1 | 2 |
| mfrbi1[c]--mfrbi1[c] | 1 | 1 |
| mfrbi2[c]--mfrbi2[c] | 1 | 1 |
| mfrbi3[c]--mfrbi3[c] | 1 | 1 |
| mfrbi4[c]--mfrbi4[c] | 1 | 1 |
| mi3p_D[c]--mi3p-D[c] | 1 | 1 |
| nac[e]--nac[e] | 1 | 2 |
| nad[c]--nad[c] | 30 | 30 |
| nh4[c]--nh4[c] | 20 | 11 |
| phom[c]--phom[c] | 1 | 1 |
| prpp[c]--prpp[c] | 7 | 14 |
| ribflv[c]--ribflv[c] | 3 | 3 |
| sec[c]--sec[c] | 1 | 1 |
| trnaglu[c]--trnaglu[c] | 1 | 1 |
| uacgam[c]--uacgam[c] | 1 | 2 |
| udpg[c]--udpg[c] | 3 | 4 |
| udpgal[c]--udpgal[c] | 1 | 2 |
| udpglcur[c]--udpglcur[c] | 1 | 2 |
| xtp[c]--xtp[c] | 1 | 1 |
| M. barkeri | in | out |
| iMG746 | degree | degree |
| 3hdggpgp[c]--2-O-3-hydroxy-geranyl-3-O-geranyl-sn-glycerol-1-phospho-3- | 1 | 1 |
| sn-glycerol-phosphate | | |
| 5aprbu[c]--5-Amino-6-5-phosphoribitylamino-uracil | 1 | 1 |
| 5pr5hbz[c]--N1-5-Phospho-alpha-D-ribosyl-5-hydroxybenzimidazole | 1 | 1 |
| 6hmhptpp[c]--6-hydroxymethyl-dihydropterin-pyrophosphate | 1 | 2 |
| 7mhp[c]--7-mercaptoheptanoic-acid | 1 | 1 |
| accoa[c]--Acetyl-CoA | 5 | 14 |
| agdpgpi[c]--N-Acetyl-D-glucosaminyl-archaetidylinositol | 1 | 1 |
| agm[c]--Agmatine | 1 | 1 |
| atp[c]--ATP | 31 | 114 |
| cbl1[c]--Cob-l-alamin | 1 | 1 |
| cbl1[e]--Cob-l-alamin | 1 | 2 |
| cbl1hbi[e]--Cob-l-alamin-HBI | 1 | 2 |
| cmp[c]--CMP | 9 | 2 |
| cu2[c]--Cu2 | 1 | 1 |
| cu2[e]--Cu2 | 1 | 1 |
| dcamp[c]--N6-1-2-Dicarboxyethyl-AMP | 1 | 1 |
| dggpgp[c]--2-3-di-O-geranyl-sn-glycerol-1-phospho-3-sn-glycerol- | 1 | 1 |
| phosphate | | |
| dhadrp[c]--7-8-dihydropterin-6-ylmethyl-1-4-aminophenyl-1-deoxy-D-ribitol- | 1 | 1 |
| 5-phosphate | | |
| dhap[c]--Dihydroxyacetone-phosphate | 4 | 6 |
| dhnpt[c]--Dihydroneopterin | 1 | 1 |
| dhp23cp[c]--7-8-dihydronepterin-2-3-cyclicphosphate | 1 | 1 |
| dhpmp[c]--Dihydroneopterin-monophosphate | 1 | 1 |
| dhpt[c]--Dihydropteroate | 1 | 1 |
| dhrfap[c]--7-8-dihydropterin-6-ylmethyl-4-B-D-ribofuranosyl-aminobenzene- | 1 | 1 |
| 5-phosphate | | |
| dkfp[c]--6-deoxy-5-ketofructose-1-phosphate | 2 | 3 |
| dohau[c]--3-7-dideoxy-D-threo-hepto-2-amino-6-ulosonate | 1 | 1 |
| dohdu[c]--3-7-dideoxy-D-threo-hepto-2-6-diulosonate | 1 | 1 |
| dpgpi[c]--2-3-O-phytanyl-sn-glycero-1-phospho-myo-inositol | 1 | 2 |
| dscl[c]--dihydrosirohydrochlorin | 1 | 2 |
| dtmp[c]--dTMP | 3 | 2 |
| eig3p[c]--D-erythro-1-Imidazol-4-yl-glycerol-3-phosphate | 1 | 1 |
| f430p3[c]--coenzyme-f430-precursor-3 | 1 | 1 |
| glu1sa[c]--L-Glutamate-1-semialdehyde | 1 | 1 |
| glu-L[c]--L-Glutamate | 31 | 36 |
| glycogen[c]--glycogen | 1 | 3 |
| gmp[c]--GMP | 7 | 2 |
| gtp[c]--GTP | 2 | 22 |
| h2mpt[c]--7-8-dihydromethanopterin | 1 | 1 |
| h2o[c]--H2O | 66 | 153 |
| h[c]--H | 237 | 168 |
| hisp[c]--L-Histidinol-phosphate | 1 | 1 |
| hmbil[c]--Hydroxymethylbilane | 1 | 1 |
| iasp[c]--Iminoaspartate | 2 | 1 |
| imp[c]--IMP | 2 | 4 |
| ipdp[c]--Isopentenyl-diphosphate | 2 | 9 |
| nac[e]--Nicotinate | 1 | 2 |
| nad[c]--Nicotinamide-adenine-dinucleotide | 25 | 25 |
| phom[c]--O-Phospho-L-homoserine | 1 | 1 |
| pran[c]--N-5-Phospho-D-ribosyl-anthranilate | 1 | 1 |
| prbamp[c]--1-5-Phosphoribosyl-AMP | 1 | 1 |
| prbatp[c]--1-5-Phosphoribosyl-ATP | 1 | 1 |
| prfp[c]--1-5-Phosphoribosyl-5-5-phosphoribosylamino- | 1 | 1 |
| methylideneaminoimidazole-4-carboxamide | | |
| prpp[c]--5-Phospho-alpha-D-ribose-1-diphosphate | 4 | 10 |
| r15bp[c]--D-Ribose-1-5-bisphosphate | 1 | 1 |
| rb15bp[c]--D-Ribulose-1-5-bisphosphate | 1 | 1 |
| ribflv[c]--Riboflavin | 4 | 2 |
| ru5p-D[c]--D-Ribulose-5-phosphate | 2 | 3 |
| sec[c]--sulfoethylcysteine | 1 | 1 |
| trnaglu[c]--tRNA-Glu | 1 | 1 |
| uacgam[c]--UDP-N-acetyl-D-glucosamine | 2 | 2 |
| udpg[c]--UDPglucose | 3 | 3 |
| udpgal[c]--UDPgalactose | 1 | 2 |
| udpglcur[c]--UDP-D-glucuronate | 1 | 2 |
| mi3p-D[c]--1D-myo-Inositol 3-phosphate | 1 | 2 |
| fapy[c]--2-amino-5-formylamino-6-ribosylamino-4(3H)-pyrimidinone 50- | | |
| monophosphate | 1 | 1 |
| xtp[c]--XTP | 1 | 1 |
| udpacgal[c]--UDP-N-acetylglucosamine | 1 | 2 |
| mfrbi1[c]--methanofuran intermediate 1 | 1 | 1 |
| mfrbi2[c]--methanofuran intermediate 2 | 1 | 1 |
| mfrbi3[c]--methanofuran intermediate 3 | 1 | 1 |
| mfrbi4[c]--phosphate ester of dihydrofuran: methanofuran biosynthesis | 1 | 1 |
| intermediate 4 | | |
| M. pneumoniae | in | out |
| iJW145 | degree | degree |
| EC0103--D-Ribose[e] | 1 | 1 |
| EC0207--Pyridoxal[e] | 1 | 1 |
| EC0078--sn-Glycerol 3-phosphate[e] | 1 | 1 |
| EC0610--Pantetheine[e] | 1 | 1 |
| EC0029--Acetate[e] | 1 | 1 |
| EC0210--Nicotinate[e] | 1 | 1 |
| EC0199--Guanine[e] | 1 | 1 |
| EC0389--Folic acid[e] | 1 | 1 |
| EC9483--Phosphatidylcholine (Mpn)[e] | 1 | 1 |
| EC9324--Biomass[e] | 2 | 1 |
| EC0300--Thiamin[e] | 1 | 1 |
| EC0025--H2O2[e] | 1 | 2 |
| EC0646--Deoxycytidine[e] | 1 | 1 |
| EC0212--Riboflavin[e] | 1 | 1 |
| EC0154--(S)-Lactate[e] | 1 | 1 |
| EC0146--Thymine[e] | 1 | 1 |
| EC0132--L-Homocysteine[e] | 1 | 1 |
| C0504--2-Deoxy-D-ribose 5-phosphate | 1 | 2 |
| C0093--Dihydroxyacetone phosphate | 5 | 4 |
| C9485--5mcDNA (Mpn) | 1 | 1 |
| C9474--phosphatidic acid (Mpn) | 1 | 2 |
| C0019--S-Adenosyl-L-homocysteine | 1 | 1 |
| C0503--2-Deoxy-D-ribose 1-phosphate | 5 | 5 |
| C0001--H2O | 13 | 61 |
| C0025--H2O2 | 2 | 2 |
| C0132--L-Homocysteine | 1 | 1 |
| N. pharaonis | in | out |
| iOG654 | degree | degree |
| C04454--5-Amino-6-(5’-phosphoribitylamino)uracil | 1 | 1 |
| C00235--Dimethylallyl diphosphate | 1 | 2 |
| C00921--Dihydropteroate | 1 | 1 |
| C00129--Isopentenyl diphosphate | 2 | 8 |
| C00002--ATP | 37 | 89 |
| C00003--NAD+ | 28 | 36 |
| C05923--2,5-Diaminopyrimidine nucleoside triphosphate | 1 | 1 |
| C05922--Formamidopyrimidine nucleoside triphosphate | 1 | 1 |
| C05925--Dihydroneopterin phosphate | 1 | 1 |
| C05817--2-Succinyl-6-hydroxy-2,4-cyclohexadiene-1-carboxylate | 1 | 1 |
| C00445--5,10-Methenyltetrahydrofolate | 3 | 3 |
| C00448--trans,trans-Farnesyl diphosphate | 1 | 1 |
| CE00121--D-Ribose (E) | 1 | 2 |
| C00341--Geranyl diphosphate | 1 | 1 |
| C01102--O-Phospho-L-homoserine | 1 | 1 |
| C04895--2-Amino-4-hydroxy-6-(erythro-1,2,3-trihydroxypropyl) | 1 | 3 |
| C00353--Geranylgeranyl diphosphate | 2 | 4 |
| C04778--N1-(5-Phospho-alpha-D-ribosyl)-5,6-dimethylbenzimidazole | 1 | 1 |
| C01230--all-trans-Hexaprenyl diphosphate | 1 | 1 |
| C04216--all-trans-Heptaprenyl diphosphate | 1 | 1 |
| C04217--all-trans-Pentaprenyl diphosphate | 1 | 1 |
| C01185--Nicotinate D-ribonucleotide | 2 | 2 |
| C05430--zeta-Carotene | 1 | 1 |
| C05432--Lycopene | 1 | 1 |
| C01304--2,5-Diamino-6-hydroxy-4-(5’-phosphoribosylamino)- | 1 | 1 |
| pyrimidine | | |
| C00049--L-Aspartate | 5 | 12 |
| C05414--Phytofluene | 1 | 1 |
| C05413--Phytoene | 1 | 1 |
| C00044--GTP | 3 | 8 |
| C05840--Iminoaspartate | 1 | 1 |
| C00199--D-Ribulose 5-phosphate | 2 | 2 |
| C00054--Adenosine 3’,5’-bisphosphate | 1 | 1 |
| CE00238--Potassium (E) | 1 | 1 |
| C00064--L-Glutamine | 7 | 14 |
| C02094--beta-Carotene | 1 | 1 |
| C00068--Thiamin diphosphate | 3 | 2 |
| P. putida | in | out |
| iJP962 | degree | degree |
| ‘EC0021--Iron[e]’ | 1 | 2 |
| ‘EC0027--D-Glucose[e]’ | 1 | 3 |
| ‘EC0048--Sulfate[e]’ | 1 | 2 |
| ‘EC0065--H+[e]’ | 38 | 44 |
| ‘EC0302--Cytosine[e]’ | 1 | 2 |
| ‘EC0154--(S)-Lactate[e]’ | 1 | 2 |
| ‘C0805--D-Arabinose 5-phosphate’ | 1 | 2 |
| ‘C0058--L-Methionine’ | 3 | 3 |
| ‘C0211--Prephenate’ | 1 | 3 |
| ‘C2932--2-Amino-4-hydroxy-6-(erythro-1,2,3-trihydroxypropyl)dihydropteridine | 1 | 1 |
| triphosphate’ | | |
| ‘C9489--Acetylated Alginate’ | 1 | 1 |
| ‘C9452--R-3-hydroxy-(11Z)-octadecenoyl-ACP’ | 1 | 1 |
| ‘C3429--Undecaprenyl-diphospho-N-acetylmuramoyl-(N-acetylglucosamine)-L- | 1 | 1 |
| alanyl-D-glutamyl-meso-2,6-diaminopimeloyl-D-alanyl-D-alanine’ | | |
| ‘C3502--(R)-2,3-Dihydroxy-3-methylpentanoate’ | 1 | 1 |
| ‘C0743--Hydroxymethylbilane’ | 1 | 1 |
| ‘C9378--(R)-3-Hydroxydodecanoyl-[acyl-carrier protein]’ | 1 | 3 |
| ‘C0999--Cys-Gly’ | 1 | 1 |
| ‘C3404--Iminoaspartate’ | 1 | 1 |
| ‘C0039--L-Lysine’ | 3 | 4 |
| ‘C9609--L-Methionyl-tRNA (fMet)’ | 1 | 1 |
| ‘C9586--Rha(alpha1,3)-Rha-(alpha1,2)-Rha-(alpha1,4)-N-acetylglucosamine | 1 | 1 |
| pyrophosphorylundecaprenol’ | | |
| ‘C0286--(S)-3-Hydroxy-3-methylglutaryl-CoA’ | 1 | 2 |
| ‘C0042--Glutathione’ | 2 | 3 |
| ‘C0022--Acetyl-CoA’ | 14 | 17 |
| ‘C1143--Cadaverine’ | 1 | 1 |
| ‘C0751--4-Maleylacetoacetate’ | 1 | 1 |
| ‘C0606--L-Arogenate’ | 1 | 1 |
| ‘C2158--2-Methylprop-2-enoyl-CoA’ | 1 | 1 |
| ‘C0082--L-Cysteine’ | 4 | 3 |
| ‘C9431--R-3-hydroxy-hexadecanoyl-ACP’ | 1 | 1 |
| ‘C9443--R-3-hydroxy-(7Z)-tetradecenoyl-ACP’ | 1 | 1 |
| ‘C9602--tRNA(Asn)’ | 1 | 1 |
| ‘C0007--Oxygen’ | 2 | 22 |
| ‘C0099--D-Ribose 5-phosphate’ | 5 | 4 |
| ‘C9490--Alginate’ | 1 | 1 |
| ‘C9446--R-3-hydroxy-(9Z)-hexadecenoyl-ACP’ | 1 | 1 |
| ‘C0854--3-(4-Hydroxyphenyl)pyruvate’ | 2 | 2 |
| ‘C0797--O-Phospho-L-homoserine’ | 1 | 1 |
| ‘C9386--(R)-3-Hydroxyhexanoyl-[acp]’ | 1 | 1 |
| ‘C2184--Indoleglycerol phosphate’ | 1 | 1 |
| ‘C3451--Dihydroneopterin phosphate’ | 1 | 1 |
| ‘C9389--trans-Dec-2-enoyl-[acyl-carrier protein]’ | 1 | 2 |
| ‘C2037--3-Methylglutaconyl-CoA’ | 2 | 1 |
| ‘C9392--UDP-3-O-(3OH-C10:0)-N-acetylglucosamine’ | 1 | 1 |
| ‘C9576--Pseudomonas LPS core precursor 4 + KDO2-lipidA’ | 1 | 1 |
| ‘C0429--4-Aminobutanal’ | 1 | 3 |
| ‘C0795--L-Histidinol phosphate’ | 1 | 2 |
| ‘C3728--ADP-D-glycero-D-manno-heptose’ | 1 | 1 |
| ‘C8533--4-amino-4-deoxychorismate’ | 1 | 1 |
| ‘C3498--(S)-3-Hydroxyisobutyryl-CoA’ | 1 | 1 |
| ‘C0037--UDP-N-acetyl-D-glucosamine’ | 2 | 3 |
| ‘C9257--R-3-hydroxy-myristoyl-ACP’ | 1 | 1 |
| ‘C1751--Phosphoribosyl-AMP’ | 1 | 1 |
| ‘C0422--Homogentisate’ | 1 | 1 |
| ‘C9579--Pseudomonas LPS core precursor 7 + KDO2-lipidA’ | 1 | 1 |
| ‘C0041--L-Aspartate’ | 6 | 15 |
| ‘C0052--L-Glutamine’ | 2 | 14 |
| ‘C2701--3-Deoxy-D-manno-octulosonate 8-phosphate’ | 1 | 1 |
| ‘C0770--4-Fumarylacetoacetate’ | 1 | 1 |
| ‘C2309--(S)-4-Amino-5-oxopentanoate’ | 1 | 1 |
| ‘C9439--R-3-hydroxy-(5Z)-dodecenoyl-ACP’ | 1 | 1 |
| ‘C9393--UDP-3-O-(3OH-C10:0)-D-glucosamine’ | 1 | 1 |
| ‘C0095--PQQ’ | 1 | 1 |
| ‘C2823--2-Dehydro-3-deoxy-D-arabino-heptonate 7-phosphate’ | 1 | 1 |
| ‘C0065--H+’ | 258 | 187 |
| ‘C0002--ATP’ | 30 | 132 |
| ‘C2933--5-(5-Phospho-D-ribosylaminoformimino)-1-(5-phosphoribosyl)- | 1 | 1 |
| imidazole-4-carboxamide’ | | |
| ‘C0147--Agmatine’ | 1 | 1 |
| ‘C0228--D-Erythrose 4-phosphate’ | 2 | 3 |
| ‘C9383--(R)-3-Hydroxybutanoyl-[acyl-carrier protein]’ | 2 | 2 |
| ‘C9417--UDP-2-NH-(3OH-C12:0),3-O-(3OH-C10:0)glucosamine’ | 1 | 2 |
| ‘C2691--5-Amino-6-(5″-phosphoribitylamino)uracil’ | 1 | 1 |
| ‘C9580--Pseudomonas LPS core precursor 8 + KDO2-lipidA’ | 1 | 1 |
| ‘C2809--D-erythro-1-(Imidazol-4-yl)glycerol 3-phosphate’ | 1 | 1 |
| ‘C0383--ADPglucose’ | 1 | 1 |
| ‘C0696--L-2-Aminoadipate’ | 1 | 1 |
| ‘C2612--N-(5-Phospho-D-ribosyl)anthranilate’ | 1 | 2 |
| ‘C9577--Pseudomonas LPS core precursor 5 + KDO2-lipidA’ | 1 | 1 |
| ‘C0092--2-Oxobutanoate’ | 2 | 1 |
| ‘C0123--GMP’ | 3 | 3 |
| ‘C8637--D-Glycero-D-manno-heptose 1,7-bisphosphate’ | 1 | 1 |
| ‘C9587--Pseudomonas Common O Polysaccharide’ | 1 | 1 |
| ‘C0306--Guanosine’ | 1 | 1 |
| ‘C1205--Xanthosine’ | 1 | 1 |
| ‘C2920--2-Amino-4-hydroxy-6-(D-erythro-1,2,3-trihydroxypropyl)-7,8- | 1 | 1 |
| dihydropteridine’ | | |
| ‘C0075--Catechol’ | 1 | 1 |
| ‘C9494--tRNA(Asp)’ | 1 | 1 |
| ‘C0491--Xanthosine 5″-phosphate’ | 2 | 3 |
| ‘C9382--(R)-3-Hydroxyoctanoyl-[acyl-carrier protein]’ | 1 | 1 |
| ‘C0318--L-Pipecolate’ | 1 | 1 |
| ‘C0038--GTP’ | 2 | 8 |
| ‘C1480--2-Methylcitrate’ | 1 | 1 |
| ‘C9581--Pseudomonas LPS core precursor 9 + KDO2-lipidA’ | 1 | 1 |
| S. aureus | in | out |
| iSB619 | degree | degree |
| 23dhmb[c]--(R)-2,3-Dihydroxy-3-methylbutanoate | 1 | 1 |
| 23dhmp[c]--(R)-2,3-Dihydroxy-3-methylpentanoate | 1 | 1 |
| 2dda7p[c]--2-Dehydro-3-deoxy-D-arabino-heptonate 7-phosphate | 1 | 1 |
| 3ig3p[c]--C’-(3-Indolyl)-glycerol 3-phosphate | 1 | 1 |
| 3php[c]--3-Phosphohydroxypyruvate | 1 | 1 |
| 4adcho[c]--4-amino-4-deoxychorismate | 1 | 1 |
| 5aprbu[c]--5-Amino-6-(5’-phosphoribitylamino)uracil | 1 | 1 |
| 5mdr1p[c]--5-Methylthio-5-deoxy-D-ribose 1-phosphate | 2 | 1 |
| 5mdru1p[c]--5-Methylthio-5-deoxy-D-ribulose 1-phosphate | 1 | 2 |
| 5mta[c]--5-Methylthioadenosine | 1 | 1 |
| ACP[c]--acyl carrier protein | 3 | 3 |
| acnam[c]--N-Acetylneuraminate | 1 | 1 |
| acnam[e]--N-Acetylneuraminate | 1 | 2 |
| agm[c]--Agmatine | 1 | 1 |
| ahdt[c]--2-Amino-4-hydroxy-6-(erythro-1,2,3- | 1 | 3 |
| trihydroxypropyl)dihydropteridine triphosphate | | |
| csn[c]--Cytosine | 1 | 1 |
| csn[e]--Cytosine | 1 | 2 |
| cyst-L[c]--L-Cystathionine | 1 | 1 |
| dcamp[c]--N6-(1,2-Dicarboxyethyl)-AMP | 1 | 1 |
| dhap[c]--Dihydroxyacetone phosphate | 7 | 5 |
| dhnpt[c]--Dihydroneopterin | 2 | 1 |
| dhpmp[c]--Dihydroneopterin monophosphate | 1 | 1 |
| dmpp[c]--Dimethylallyl diphosphate | 1 | 2 |
| e4p[c]--D-Erythrose 4-phosphate | 2 | 4 |
| eig3p[c]--D-erythro-1-(Imidazol-4-yl)glycerol 3-phosphate | 1 | 1 |
| gln-L[c]--L-Glutamine | 1 | 18 |
| glu1sa[c]--L-Glutamate 1-semialdehyde | 1 | 1 |
| grdp[c]--Geranyl diphosphate | 1 | 1 |
| gua[e]--Guanine | 1 | 2 |
| h2o[c]--H2O | 70 | 124 |
| h[c]--H+ | 219 | 132 |
| h[e]--H+ | 25 | 35 |
| hisp[c]--L-Histidinol phosphate | 1 | 1 |
| hmbil[c]--Hydroxymethylbilane | 1 | 1 |
| imacp[c]--3-(Imidazol-4-yl)-2-oxopropyl phosphate | 1 | 1 |
| ipdp[c]--Isopentenyl diphosphate | 2 | 3 |
| malt[c]--Maltose | 1 | 1 |
| malt[e]--Maltose | 1 | 2 |
| nac[e]--Nicotinate | 1 | 2 |
| nad[c]--Nicotinamide adenine dinucleotide | 33 | 47 |
| ncam[c]--Nicotinamide | 1 | 1 |
| ncam[e]--Nicotinamide | 1 | 2 |
| o2[e]--O2 | 1 | 2 |
| pep[c]--Phosphoenolpyruvate | 3 | 14 |
| phom[c]--O-Phospho-L-homoserine | 1 | 1 |
| prbamp[c]--1-(5-Phosphoribosyl)-AMP | 1 | 1 |
| prbatp[c]--1-(5-Phosphoribosyl)-ATP | 1 | 1 |
| prfp[c]--1-(5-Phosphoribosyl)-5-[(5- | 1 | 1 |
| phosphoribosylamino)methylideneamino]imidazole-4-carboxamide | | |
| pser-L[c]--O-Phospho-L-serine | 1 | 1 |
| rib-D[e]--D-Ribose | 1 | 2 |
| ru5p-D[c]--D-Ribulose 5-phosphate | 3 | 3 |
| thm[e]--Thiamin | 1 | 2 |
| tre6p[c]--alpha,alpha’-Trehalose 6-phosphate | 1 | 1 |
| uacgam[c]--UDP-N-acetyl-D-glucosamine | 3 | 5 |
| Synechocystis sp. | in | out |
| iJN678 | degree | degree |
| atp[c]--ATP | 29 | 114 |
| h[c]--H+ | 233 | 194 |
| nad[c]--Nicotinamide adenine dinucleotide | 20 | 28 |
| 34hpp[c]--3-(4-Hydroxyphenyl)pyruvate | 2 | 2 |
| tdec2eACP[c]--trans-Dec-2-enoyl-[acyl-carrier protein] | 2 | 2 |
| 3hddecACP[c]--(R)-3-Hydroxydodecanoyl-[acyl-carrier protein] | 1 | 2 |
| tddec2eACP[c]--trans-Dodec-2-enoyl-[acyl-carrier protein] | 1 | 1 |
| 3hcddec5eACP[c]--(R)-3-hydroxy-cis-dodec-5-enoyl-[acyl-carrier protein] | 1 | 1 |
| t3c5ddeceACP[c]--trans-3-cis-5-dodecenoyl-[acyl-carrier protein] | 1 | 1 |
| 3hmrsACP[c]--(R)-3-Hydroxytetradecanoyl-[acyl-carrier protein] | 2 | 3 |
| tmrs2eACP[c]--trans-Tetradec-2-enoyl-[acyl-carrier protein] | 1 | 1 |
| 3hcmrs7eACP[c]--(R)-3-hydroxy-cis-myristol-7-eoyl-[acyl-carrier protein] | 1 | 1 |
| t3c7mrseACP[c]--trans-3-cis-7-myristoleoyl-[acyl-carrier protein] | 1 | 1 |
| 3hpalmACP[c]--R-3-hydroxypalmitoyl-[acyl-carrier protein] | 1 | 2 |
| tpalm2eACP[c]--trans-Hexadec-2-enoyl-[acyl-carrier protein] | 1 | 1 |
| 3hcpalm9eACP[c]--(R)-3-hydroxy-cis-palm-9-eoyl-[acyl-carrier protein] | 1 | 1 |
| t3c9palmeACP[c]--trans-3-cis-9-palmitoleoyl-[acyl-carrier protein] | 1 | 1 |
| 3hoctaACP[c]--(R)-3-Hydroxyoctadecanoyl-[acyl-carrier protein] | 1 | 1 |
| toctd2eACP[c]--trans-octadec-2-enoyl-[acyl-carrier protein] | 1 | 1 |
| 3hcvac11eACP[c]--(R)-3-hydroxy-cis-vacc-11-enoyl-[acyl-carrier protein] | 1 | 1 |
| t3c11vaceACP[c]--trans-3-cis-11-vacceoyl-[acyl-carrier protein] | 1 | 1 |
| 3haACP[c]--(3R)-3-Hydroxyacyl-[acyl-carrier protein] | 1 | 2 |
| but2eACP[c]--But-2-enoyl-[acyl-carrier protein] | 1 | 1 |
| 3hhexACP[c]--(R)-3-Hydroxyhexanoyl-[acyl-carrier protein] | 1 | 2 |
| thex2eACP[c]--trans-Hex-2-enoyl-[acyl-carrier protein] | 1 | 1 |
| 3hoctACP[c]--(R)-3-Hydroxyoctanoyl-[acyl-carrier protein] | 1 | 2 |
| toct2eACP[c]--trans-Oct-2-enoyl-[acyl-carrier protein] | 1 | 1 |
| 3ocddec5eACP[c]--3-oxo-cis-dodec-5-enoyl-[acyl-carrier protein] | 1 | 1 |
| 3omrsACP[c]--3-Oxotetradecanoyl-[acyl-carrier protein] | 1 | 1 |
| 3ocmrs7eACP[c]--3-oxo-cis-myristol-7-eoyl-[acyl-carrier protein] | 1 | 1 |
| 3ocpalm9eACP[c]--3-oxo-cis-palm-9-eoyl-[acyl-carrier protein] | 1 | 1 |
| 30octdACP[c]--3-Oxooctadecanoyl-[acyl-carrier protein] | 1 | 1 |
| 3ocvac11eACP[c]--3-oxo-cis-vacc-11-enoyl-[acyl-carrier protein] | 1 | 1 |
| aacoa[c]--Acetoacetyl-CoA | 1 | 1 |
| 3hbcoa-R[c]--(R)-3-Hydroxybutyryl-CoA | 1 | 1 |
| gtp[c]--GTP | 2 | 9 |
| agdpcbi[c]--Adenosine-GDP-cobinamide | 1 | 1 |
| accoa[c]--Acetyl-CoA | 7 | 14 |
| pyr[c]--Pyruvate | 13 | 12 |
| alac-S[c]--(S)-2-Acetolactate | 1 | 1 |
| gln-L[c]--L-Glutamine | 2 | 19 |
| adprib[c]--ADPribose | 1 | 1 |
| prpp[c]--5-Phospho-alpha-D-ribose 1-diphosphate | 2 | 9 |
| asp-L[c]--L-Aspartate | 4 | 13 |
| agm[c]--Agmatine | 1 | 1 |
| pa180[c]--1,2-dioctadecanoyl-sn-glycerol 3-phosphate | 1 | 2 |
| gcald[c]--Glycolaldehyde | 1 | 1 |
| pran[c]--N-(5-Phospho-D-ribosyl)anthranilate | 1 | 1 |
| 5apru[c]--5-Amino-6-(5’-phosphoribosylamino)uracil | 1 | 1 |
| 5aprbu[c]--5-Amino-6-(5’-phosphoribitylamino)uracil | 1 | 1 |
| 4pasp[c]--4-Phospho-L-aspartate | 1 | 1 |
| iasp[c]--Iminoaspartate | 1 | 1 |
| h[p]--H+ | 17 | 20 |
| prbatp[c]--1-(5-Phosphoribosyl)-ATP | 1 | 1 |
| amet[c]--S-Adenosyl-L-methionine | 4 | 16 |
| 2p4c2me[c]--2-phospho-4-(cytidine 5’-diphospho)-2-C-methyl-D-erythritol | 1 | 1 |
| 3psme[c]--5-O-(1-Carboxyvinyl)-3-phosphoshikimate | 1 | 1 |
| hgbam[c]--Hydrogenobyrinate a,c diamide | 1 | 1 |
| cys-L[c]--L-Cysteine | 1 | 5 |
| cmp[c]--CMP | 12 | 2 |
| ru5p-D[c]--D-Ribulose 5-phosphate | 3 | 4 |
| e4p[c]--D-Erythrose 4-phosphate | 3 | 4 |
| 2dda7p[c]--2-Dehydro-3-deoxy-D-arabino-heptonate 7-phosphate | 1 | 1 |
| 23dhmb[c]--(R)-2,3-Dihydroxy-3-methylbutanoate | 1 | 1 |
| 23dhmp[c]--(R)-2,3-Dihydroxy-3-methylpentanoate | 1 | 1 |
| 23dhdp[c]--2,3-Dihydrodipicolinate | 1 | 1 |
| dhpt[c]--Dihydropteroate | 1 | 1 |
| dhnpt[c]--Dihydroneopterin | 1 | 1 |
| 3dhq[c]--3-Dehydroquinate | 1 | 1 |
| 3dhsk[c]--3-Dehydroshikimate | 1 | 1 |
| h2mb4p[c]--1-hydroxy-2-methyl-2-(E)-butenyl 4-diphosphate | 1 | 2 |
| dmpp[c]--Dimethylallyl diphosphate | 2 | 2 |
| dhpmp[c]--Dihydroneopterin monophosphate | 1 | 1 |
| ahdt[c]--2-Amino-4-hydroxy-6-(erythro-1,2,3-trihydroxypropyl)dihydropteridine | 1 | 1 |
| triphosphate | | |
| pchlld[c]--Protochlorophyllide | 1 | 1 |
| 2dhp[c]--2-Dehydropantoate | 1 | 1 |
| g3p[c]--Glyceraldehyde 3-phosphate | 7 | 7 |
| dvpchild[c]--Divinylprotochlorophyllide | 2 | 1 |
| dxyl5p[c]--1-deoxy-D-xylulose 5-phosphate | 1 | 1 |
| ipdp[c]--Isopentenyl diphosphate | 2 | 6 |
| glu1sa[c]--L-Glutamate 1-semialdehyde | 1 | 1 |
| glu5p[c]--L-Glutamate 5-phosphate | 1 | 1 |
| 6pgl[c]--6-phospho-D-glucono-1,5-lactone | 2 | 1 |
| glycogen[c]--glycogen | 1 | 3 |
| adpgic[c]--ADPglucose | 1 | 1 |
| glutrna[c]--L-Glutamyl-tRNA(Glu) | 1 | 1 |
| trnaglu[c]--tRNA (Glu) | 1 | 1 |
| grdp[c]--Geranyl diphosphate | 1 | 1 |
| hisp[c]--L-Histidinol phosphate | 1 | 1 |
| hmbil[c]--Hydroxymethylbilane | 1 | 1 |
| phom[c]--O-Phospho-L-homoserine | 1 | 1 |
| imacp[c]--3-(Imidazol-4-yl)-2-oxopropyl phosphate | 1 | 1 |
| eig3p[c]--D-erythro-1-(Imidazol-4-yl)glycerol 3-phosphate | 1 | 1 |
| 3ig3p[c]--C’-(3-Indolyl)-glycerol 3-phosphate | 1 | 1 |
| 2ahbut[c]--(S)-2-Aceto-2-hydroxybutanoate | 1 | 1 |
| u23ga[c]--UDP-2,3-bis(3-hydroxytetradecanoyl)glucosamine | 1 | 2 |
| lycop[c]--Lycopene | 1 | 1 |
| sbzcoa[c]--O-Succinylbenzoyl-CoA | 1 | 1 |
| thmpp[c]--Thiamine diphosphate | 1 | 1 |
| ssaltpp[c]--Succinate semialdehyde-thiamin diphosphate anion | 1 | 1 |
| pap[c]--Adenosine 3’,5’-bisphosphate | 1 | 1 |
| pre6a[c]--Precorrin 6A | 1 | 1 |
| 2pglyc[c]--2-Phosphoglycolate | 1 | 1 |
| pgp160[c]--Phosphatidylglycerophosphate (dihexadecanoyl, n-C16_0) | 1 | 1 |
| pgp161[c]--Phosphatidylglycerophosphate (dihexadec-9-enoyl, n-C16_1) | 1 | 1 |
| pgp180[c]--Phosphatidylglycerophosphate (dioctadecanoyl, n-C18_0) | 1 | 1 |
| pgp181[c]--Phosphatidylglycerophosphate (dioctadec-11-enoyl, n-C18_1) | 1 | 1 |
| pgp181_9[c]--Phosphatidylglycerophosphate (dioctadec-9-enoyl, n-C18_1) | 1 | 1 |
| pgp182_9_12[c]--Phosphatidylglycerophosphate (dioctadec-9-12-dienoyl, n- | 1 | 1 |
| C18_2) | | |
| pgp183_6_9_12[c]--Phosphatidylglycerophosphate (dioctadec-6-9-12-trienoyl, n- | 1 | 1 |
| C18_3) | | |
| pgp183_9_12_15[c]--Phosphatidylglycerophosphate (dioctadec-9-12-15-trienoyl, | 1 | 1 |
| n-C18_3) | | |
| pgp184_6_9_12_15[c]--Phosphatidylglycerophosphate (dioctadec-6-9-12-15- | 1 | 1 |
| tetraenoyl, n-C18_4) | | |
| PHB[c]--PHB granule | 1 | 1 |
| prephytedp[c]--Prephytoene diphosphate | 1 | 1 |
| peptido_syn[c]--Peptidoglycan subunit of Synechocystis sp. PCC8603 | 1 | 1 |
| uaagmda[c]--Undecaprenyl-diphospho-N-acetylmuramoyl-(N- | 1 | 1 |
| acetylglucosamine)-L-ala-D-glu-meso-2,6-diaminopimeloyl-D-ala-D-ala | | |
| udcpdp[c]--Undecaprenyl diphosphate | 2 | 4 |
| decdp[c]--all trans Decaprenyl diphosphate | 1 | 1 |
| prbamp[c]--1-(5-Phosphoribosyl)-AMP | 1 | 1 |
| prfp[c]--1-(5-Phosphoribosyl)-5-[(5- | 1 | 1 |
| phosphoribosylamino)methylideneamino]imidazole-4-carboxamide | | |
| rb15bp[c]--D-Ribulose 1,5-bisphosphate | 1 | 2 |
| 2shchc[c]--2-Succinyl-6-hydroxy-2,4-cyclohexadiene-1-carboxylate | 1 | 1 |
| thr-L[c]--L-Threonine | 1 | 5 |
| 2obut[c]--2-Oxobutanoate | 1 | 1 |
| u3hga[c]--UDP-3-O-(3-hydroxytetradecanoyl)-D-glucosamine | 1 | 1 |
| u3aga[c]--UDP-3-O-(3-hydroxytetradecanoyl)-N-acetylglucosamine | 1 | 2 |
| uaccg[c]--UDP-N-acetyl-3-O-(1-carboxyvinyl)-D-glucosamine | 1 | 1 |
| 4adcho[c]--4-amino-4-deoxychorismate | 1 | 1 |
| fru[p]--D-Fructose | 1 | 2 |
| cynt[p]--Cyanate | 1 | 2 |
| cytd[c]--Cytidine | 1 | 1 |
| dna5mtc[c]--DNA 5-methylcytosine | 1 | 1 |
| 5mdru1p[c]--5-Methylthio-5-deoxy-D-ribulose 1-phosphate | 1 | 1 |
| 5mdr1p[c]--5-Methylthio-5-deoxy-D-ribose 1-phosphate | 1 | 1 |
| T. maritima | in | out |
| iTZ479 | degree | degree |
| 2dda7p[c]--2-Dehydro-3-deoxy-D-arabino-heptonate 7-phosphate | 1 | 1 |
| 2p4c2me[c]--2-phospho-4-(cytidine 5’-diphospho)-2-C-methyl-D-erythritol | 1 | 1 |
| 3ig3p[c]--C’-(3-Indolyl)-glycerol 3-phosphate | 2 | 2 |
| 4adcho[c]--4-amino-4-deoxychorismate | 1 | 1 |
| 5aprbu[c]--5-Amino-6-(5’-phosphoribitylamino)uracil | 1 | 1 |
| 6pgl[c]--6-phospho-D-glucono-1,5-lactone | 1 | 1 |
| adpglc[c]--ADPglucose | 1 | 1 |
| ahdt[c]--2-Amino-4-hydroxy-6-(erythro-1,2,3-trihydroxypropyl) dihydropteridine | 1 | 2 |
| triphosphate | | |
| atp[c]--ATP | 32 | 122 |
| cell4[c]--cellulose (n = 4 repeating units) | 1 | 1 |
| csn[c]--Cytosine | 1 | 1 |
| damp[c]--dAMP | 1 | 1 |
| datp[c]--dATP | 2 | 3 |
| dcaACP[c]--Decanoyl-ACP (n-C10:0ACP) | 1 | 2 |
| dcmp[c]--dCMP | 3 | 3 |
| dgmp[c]--dGMP | 1 | 1 |
| dgtp[c]--dGTP | 2 | 3 |
| dhnpt[c]--Dihydroneopterin | 2 | 1 |
| dhpmp[c]--Dihydroneopterin monophosphate | 1 | 1 |
| dhpt[c]--Dihydropteroate | 1 | 1 |
| dip_d_d[c]--di-myo-inositol 1,1’ phosphate | 1 | 1 |
| dmpp[c]--Dimethylallyl diphosphate | 1 | 2 |
| dtmp[c]--dTMP | 4 | 2 |
| dutp[c]--dUTP | 1 | 1 |
| e4p[c]--D-Erythrose 4-phosphate | 2 | 3 |
| eig3p[c]--D-erythro-1-(Imidazol-4-yl)glycerol 3-phosphate | 1 | 1 |
| gam6p[c]--D-Glucosamine 6-phosphate | 2 | 2 |
| gln_L[c]--L-Glutamine | 1 | 12 |
| grdp[c]--Geranyl diphosphate | 1 | 1 |
| h[c]--H+ | 195 | 93 |
| h2mb4p[c]--1-hydroxy-2-methyl-2-(E)-butenyl 4-diphosphate | 1 | 1 |
| hisp[c]--L-Histidinol phosphate | 1 | 2 |
| iasp[c]--Iminoaspartate | 1 | 1 |
| indole[c]--Indole | 1 | 1 |
| ipdp[c]--Isopentenyl diphosphate | 2 | 4 |
| lcts[c]--Lactose | 1 | 1 |
| lmn2[c]--Laminaribiose | 1 | 1 |
| maltttr[c]--Maltotetraose | 1 | 1 |
| man[c]--D-Mannose | 9 | 2 |
| manttr[c]--mannotetraose | 1 | 1 |
| met_L[c]--L-Methionine | 1 | 2 |
| nad[c]--Nicotinamide adenine dinucleotide | 22 | 26 |
| nadp[c]--Nicotinamide adenine dinucleotide phosphate | 25 | 15 |
| phom[c]--O-Phospho-L-homoserine | 1 | 1 |
| prbamp[c]--1-(5-Phosphoribosyl)-AMP | 1 | 1 |
| prbatp[c]--1-(5-Phosphoribosyl)-ATP | 1 | 2 |
| prfp[c]--1-(5-Phosphoribosyl)-5-[(5- | 1 | 1 |
| phosphoribosylamino)methylideneamino]imidazole-4-carboxamide | | |
| pydx5p[c]--Pyridoxal 5’-phosphate | 1 | 1 |
| raffin[c]--Raffinose | 1 | 1 |
| ru5p_D[c]--D-Ribulose 5-phosphate | 3 | 3 |
| sprm[c]--Spermine | 1 | 1 |
| sucr[c]--Sucrose | 1 | 1 |
| tre[c]--Trehalose | 1 | 1 |
| uaaGgla[c]--Undecaprenyl-diphospho-N-acetylmuramoyl-(N-acetylglucosamine)- | 1 | 1 |
| L-alanyl-gamma-D-glutamyl-L-lysyl-D-alanyI-D-alanine | | |
| udcpdp[c]--Undecaprenyl diphosphate | 2 | 2 |
| cell4[e]--cellulose (n = 4 repeating units) | 2 | 2 |
| cell6[e]--cellulose (n = 6 repeating units) | 1 | 2 |
| csn[e]--Cytosine | 1 | 2 |
| galman4[e]--galactomannan(n = 4 repeat units mannose, alpha-1,4 man) | 3 | 2 |
| galman6[e]--galactomannan(n = 6 repeat units mannose, alpha-1,4 man) | 3 | 2 |
| glcman4[e]--glucomannan (n = 4 repeat units, glc beta-1,4 man) | 3 | 2 |
| glcman6[e]--glucomannan (n = 6 repeat units, glc beta-1,4 man) | 3 | 2 |
| glucan4[e]--beta-1,3/1,4-glucan (Barley, n = 4, Glc beta1->3,4 Glc) | 3 | 2 |
| glucan6[e]--beta-1,3/1,4-glucan (Barley, n = 6, Glc beta1->3,4 Glc) | 3 | 2 |
| glyc[e]--Glycerol | 1 | 2 |
| h[e]--H+ | 8 | 10 |
| lcts[e]--Lactose | 1 | 2 |
| lmn2[e]--Laminaribiose | 2 | 2 |
| maltttr[e]--Maltotetraose | 1 | 2 |
| manttr[e]--mannotetraose | 1 | 2 |
| raffin[e]--Raffinose | 1 | 2 |
| sucr[e]--Sucrose | 1 | 2 |
| tre[e]--Trehalose | 1 | 2 |
| xan[e]--Xanthine | 1 | 2 |
| Y. pestis | in | out |
| iPC815 | degree | degree |
| 2dr1p[c]--2-Deoxy-D-ribose 1-phosphate | 4 | 4 |
| g3pg[p]--Glycerophosphoglycerol | 8 | 2 |
| pa141[c]--1;2-ditetradec-7-enoyl-sn-glycerol 3-phosphate | 3 | 3 |
| 5mta[c]--5-Methylthioadenosine | 1 | 1 |
| pa141[p]--1;2-ditetradec-7-enoyl-sn-glycerol 3-phosphate | 1 | 2 |
| hisp[c]--L-Histidinol phosphate | 1 | 1 |
| tdeACP[c]--cis-tetradec-7-enoyl-[acyl-carrier protein] (n-C14:1) | 3 | 4 |
| 1agpg181[p]--1-Acyl-sn-glycero-3-phosphoglycerol (n-C18:1) | 1 | 1 |
| e4p[c]--D-Erythrose 4-phosphate | 2 | 3 |
| pe180[p]--phosphatidylethanolamine (dioctadecanoyl; n-C18:0) | 1 | 2 |
| pe180[c]--phosphatidylethanolamine (dioctadecanoyl; n-C18:0) | 2 | 1 |
| 2agpe160[p]--2-Acyl-sn-glycero-3-phosphoethanolamine (n-C16:0) | 1 | 1 |
| dhpt[c]--Dihydropteroate | 1 | 1 |
| 3dhq[c]--3-Dehydroquinate | 1 | 1 |
| 1tdec7eg3p[p]--1-tetradec-7-enoyl-sn-glycerol 3-phosphate | 1 | 1 |
| met__L[c]--L-Methionine | 2 | 3 |
| pg161[p]--Phosphatidylglycerol (dihexadec-9-enoyl; n-C16:1) | 3 | 5 |
| gcald[c]--Glycolaldehyde | 1 | 1 |
| murein4px4px4p[p]--three disacharide linked murein units (tetrapeptide crosslinked | 1 | 1 |
| tetrapeptide (A2pm->D-ala) & tetrapeptide corsslinked tetrapeptide (A2pm->D-ala)) | | |
| (middle of chain) | | |
| prbamp[c]--1-(5-Phosphoribosyl)-AMP | 1 | 1 |
| imacp[c]--3-(Imidazol-4-yl)-2-oxopropyl phosphate | 1 | 1 |
| asn__L[e]--L-Asparagine | 1 | 2 |
| asn__L[p]--L-Asparagine | 2 | 3 |
| u23ga[c]--UDP-2;3-bis(3-hydroxytetradecanoyl)glucosamine | 1 | 2 |
| lcts[p]--Lactose | 2 | 3 |
| pg140[p]--Phosphatidylglycerol (ditetradecanoyl; n-C14:0) | 3 | 5 |
| lcts[e]--Lactose | 1 | 2 |
| gtp[c]--GTP | 2 | 14 |
| g3pg[e]--Glycerophosphoglycerol | 1 | 2 |
| 2dr5p[c]--2-Deoxy-D-ribose 5-phosphate | 1 | 2 |
| 2agpg180[p]--2-Acyl-sn-glycero-3-phosphoglycerol (n-C18:0) | 1 | 1 |
| 1ddecg3p[p]--1-dodecanoyl-sn-glycerol 3-phosphate | 1 | 1 |
| pgp181[p]--Phosphatidylglycerophosphate (dioctadec-11-enoyl; n-C18:1) | 1 | 1 |
| 2tdecg3p[p]--2-tetradecanoyl-sn-glycerol 3-phosphate | 1 | 1 |
| pgp181[c]--Phosphatidylglycerophosphate (dioctadec-11-enoyl; n-C18:1) | 1 | 3 |
| cdpdhdecg[c]--CDP-1;2-dihexadecanoylglycerol | 2 | 3 |
| cddec5eACP[c]--cis-dodec-5-enoyl-[acyl-carrier protein] (n-C12:1) | 2 | 1 |
| 2tdecg3p[c]--2-tetradecanoyl-sn-glycerol 3-phosphate | 1 | 1 |
| 1tdecg3p[p]--1-tetradecanoyl-sn-glycerol 3-phosphate | 1 | 1 |
| cdpdtdec7eg[c]--CDP-1;2-ditetradec-7-enoylglycerol | 2 | 3 |
| dhnpt[c]--Dihydroneopterin | 1 | 1 |
| xtsn[p]--Xanthosine | 2 | 2 |
| xtsn[e]--Xanthosine | 1 | 2 |
| 2agpg141[p]--2-Acyl-sn-glycero-3-phosphoglycerol (n-C14:1) | 1 | 1 |
| ile_L[p]--L-Isoleucine | 2 | 3 |
| 1agpe181[p]--1-Acyl-sn-glycero-3-phosphoethanolamine (n-C18:1) | 1 | 1 |
| ile__L[e]--L-Isoleucine | 1 | 2 |
| lgt__S[c]--(R)-S-Lactoyiglutathione | 2 | 1 |
| 3hcddec5eACP[c]--(R)-3-hydroxy-cis-dodec-5-enoyl-(acyl-carrier protein] | 1 | 1 |
| uri[p]--Uridine | 2 | 3 |
| cdpdodec11eg[c]--CDP-1;2-dioctadec-11-enoylglycerol | 2 | 3 |
| phom[c]--O-Phospho-L-homoserine | 1 | 1 |
| thr__L[p]--L-Threonine | 2 | 3 |
| asp__L[e]--L-Aspartate | 1 | 2 |
| uri[e]--Uridine | 1 | 2 |
| thr__L[c]--L-Threonine | 4 | 6 |
| thr__L[e]--L-Threonine | 1 | 2 |
| asp__L[p]--L-Aspartate | 2 | 5 |
| glyc[p]--Glycerol | 6 | 6 |
| succ[e]--Succinate | 1 | 2 |
| 5mdr1p[c]--5-Methylthio-5-deoxy-D-ribose 1-phosphate | 2 | 1 |
| glyc[e]--Glycerol | 1 | 2 |
| succ[p]--Succinate | 4 | 4 |
| dhmtp[c]--1,2-Dihydroxy-5-(methylthio)pent-1-en-3-one | 2 | 2 |
| murein5px4p[p]--two disacharide linked murein units; pentapeptide crosslinked | 1 | 2 |
| tetrapeptide (A2pm->D-ala) (middle of chain) | | |
| 1agpg180[p]--1-Acyl-sn-glycero-3-phosphoglycerol (n-C18:0) | 1 | 1 |
| ura[p]--Uracil | 2 | 3 |
| ura[e]--Uraci | 1 | 2 |
| ru5p__D[c]--D-ribulose 5-phosphate | 4 | 4 |
| uaagmda[c]--Undecaprenyl-diphospho-N-acetylmuramoyl-(N-acetylglucosamine)- | 1 | 2 |
| L-ala-D-glu-meso-2;6-diaminopimeloyl-D-ala-D-ala | | |
| sl26da[c]--N-Succinyl-LL-2;6-diaminoheptanedioate | 1 | 1 |
| cdec3eACP[c]--cis-dec-3-enoyl-[acyl-carrier protein] (n-C10:1) | 1 | 2 |
| iasp[c]--Iminoaspartate | 4 | 1 |
| 1odec11eg3p[p]--1-octadec-11-enoyl-sn-glycerol 3-phosphate | 1 | 1 |
| hmbil[c]--Hydroxymethylbilane | 1 | 1 |
| accoa[c]--Acetyl-CoA | 16 | 14 |
| 5aprbu[c]--5-Amino-6-(5-phosphoribitylamino)uracil | 1 | 1 |
| hxan[p]--Hypoxanthine | 2 | 2 |
| 1hdec9eg3p[p]--1-hexadec-9-enoyl-sn-glycerol 3-phosphate | 1 | 1 |
| 1agpe161[p]--1-Acyl-sn-glycero-3-phosphoethanolamine (n-C16:1) | 1 | 1 |
| hxan[e]--Hypoxanthine | 1 | 2 |
| glyc3p[e]--Glycerol 3-phosphate | 1 | 2 |
| glyc3p[p]--Glycerol 3-phosphate | 8 | 2 |
| 1agpe140[p]--1-Acyl-sn-glycero-3-phosphoethanolamine (n-C14:0) | 1 | 1 |
| pe181[p]--phosphatidylethanolamine (dioctadec-11-enoyl; n-C18:1) | 1 | 4 |
| orn[e]--Ornithine | 1 | 2 |
| orn[p]--Ornithine | 2 | 3 |
| trp__L[p]--L-Tryptophan | 2 | 2 |
| trp__L[e]--L-Tryptophan | 1 | 2 |
| cdpdtdecg[c]--CDP-1;2-ditetradecanoylglycerol | 2 | 3 |
| akg[e]--2-Oxoglutarate | 1 | 2 |
| akg[p]--2-Oxoglutarate | 2 | 2 |
| 2agpe140[p]--2-Acyl-sn-glycero-3-phosphoethanolamine (n-C14:0) | 1 | 1 |
| pg141[p]--Phosphatidylglycerol (ditetradec-7-enoyl; n-C14:1) | 2 | 2 |
| pg160[p]--Phosphatidylglycerol (dihexadecanoyl; n-C16:0) | 3 | 5 |
| 2hdec9eg3p[p]--2-hexadec-9-enoyl-sn-glycerol 3-phosphate | 1 | 1 |
| 2hdec9eg3p[c]--2-hexadec-9-enoyl-sn-glycerol 3-phosphate | 1 | 1 |
| adpglc[c]--ADPglucose | 1 | 1 |
| nh4[p]--Ammonium | 3 | 2 |
| nh4[e]--Ammonium | 1 | 2 |
| 3php[c]--3-Phosphohydroxypyruvate | 1 | 1 |
| 2agpg181[p]--2-Acyl-sn-glycero-3-phosphoglycerol (n-C18:1) | 1 | 1 |
| lac__L[p]--L-Lactate | 2 | 2 |
| 1hdecg3p[p]--1-hexadecanoyl-sn-glycerol 3-phosphate | 1 | 1 |
| lac__D[e]--D-Lactate | 1 | 2 |
| 4mop[c]--4-Methyl-2-oxopentanoate | 1 | 1 |
| lac__D[p]--D-Lactate | 2 | 2 |
| 2hdecg3p[p]--2-hexadecanoyl-sn-glycerol 3-phosphate | 1 | 1 |
| 2hdecg3p[c]--2-hexadecanoyl-sn-glycerol 3-phosphate | 1 | 1 |
| 5aop[c]--5-Amino-4-oxopentanoate | 1 | 2 |
| lac__L[e]--L-Lactate | 1 | 2 |
| trnaglu[c]--IRNA (Glu) | 1 | 1 |
| pgp180[p]--Phosphatidylglycerophosphate (dioctadecanoyl; n-C18:0) | 1 | 1 |
| pgp180[c]--Phosphatidylglycerophosphate (dioctadecanoyl; n-C18:0) | 1 | 3 |
| dha[e]--Dihydroxyacetone | 1 | 2 |
| LalaDgluMdapDala[p]--L-alanine-D-glutamate-meso-2;6-diaminoheptanedioate-D- | 2 | 1 |
| alanine | | |
| dha[p]--Dihydroxyacetone | 2 | 2 |
| atp[c]--ATP | 30 | 187 |
| LalaDgluMdapDala[e]--L-alanine-D-glutamate-meso-2;6-diaminoheptanedioate-D- | 1 | 2 |
| alanine | | |
| etoh[p]--Ethanol | 2 | 2 |
| ara5p[c]--D-arabinose 5-phosphate | 1 | 2 |
| etoh[e]--Ethanol | 1 | 2 |
| u3hga[c]--UDP-3-O-(3-hydroxytetradecanoyl)-D-glucosamine | 1 | 1 |
| gal[e]--D-Galactose | 1 | 2 |
| pa161[c]--1;2-dihexadec-9-enoyl-sn-glycerol 3-phosphate | 3 | 3 |
| gal[p]--D-Galactose | 2 | 3 |
| pa161[p]--1;2-dihexadec-9-enoyl-sn-glycerol 3-phosphate | 1 | 2 |
| xan[e]--Xanthine | 1 | 2 |
| 1odecg3p[p]--1-octadecanoyl-sn-glycerol 3-phosphate | 1 | 1 |
| pgp120[p]--Phosphatidylglycerophosphate (didodecanoyl; n-C12:0) | 1 | 1 |
| xan[p]--Xanthine | 2 | 2 |
| dtmp[c]--dTMP | 4 | 2 |
| glyald[e]--D-Glyceraldehyde | 1 | 2 |
| for[e]--Formate | 1 | 2 |
| pgp120[c]--Phosphatidylglycerophosphate (didodecanoyl; n-C12:0) | 1 | 3 |
| glyald[p]--D-Glyceraldehyde | 2 | 2 |
| val__L[e]--L-Valine | 1 | 2 |
| val__L[p]--L-Valine | 2 | 3 |
| pheme[e]--Protoheme | 1 | 1 |
| pa180[c]--1;2-dioctadecanoyl-sn-glycerol 3-phosphate | 3 | 3 |
| pran[c]--N-(5-Phospho-D-ribosyl)anthranilate | 1 | 1 |
| pheme[c]--Protoheme | 1 | 1 |
| sl2a6o[c]--N-Succinyl-2-L-amino-6-oxoheptanedioate | 1 | 1 |
| pa180[p]--1;2-dioctadecanoyl-sn-glycerol 3-phosphate | 1 | 2 |
| pheme[p]--Protoheme | 1 | 1 |
| leu__L[e]--L-Leucine | 1 | 2 |
| leu__L[p]--L-Leucine | 2 | 3 |
| 1agpg140[p]--1-Acyl-sn-glycero-3-phosphoglycerol (n-C14:0) | 1 | 1 |
| murein4px4p4p[p]--three disacharide linked murein units (tetrapeptide crosslinked | 1 | 1 |
| tetrapeptide (A2pm->D-ala); one uncrosslinked tetrapaptide) (middle of chain) | | |
| 1agpg161[p]--1-Acyl-sn-glycero-3-phosphoglycerol (n-C16:1) | 1 | 1 |
| eig3p[c]--D-erythro-1-(Imidazol-4-yl)glycerol 3-phosphate | 1 | 1 |
| 2agpe141[p]--2-Acyl-sn-glycero-3-phosphoethanolamine (n-C14:1) | 1 | 1 |
| cdpdddecg[c]--CDP-1;2-didodecanoylglycerol | 2 | 3 |
| ac[p]--Acetate | 2 | 3 |
| g3pe[p]--sn-Glycero-3-phosphoethanolamine | 8 | 1 |
| 2dda7p[c]--2-Dehydro-3-deoxy-D-arabino-heptonate 7-phosphate | 1 | 1 |
| fum[p]--Fumarate | 2 | 4 |
| fum[e]--Fumarate | 1 | 2 |
| gdptp[c]--Guanosine 3-diphosphate 5-triphosphate | 1 | 1 |
| murein5px4px4p[p]--three disacharide linked murein units (pentapeptide | 1 | 1 |
| crosslinked tetrapeptide (A2pm->D-ala) tetrapeptide corsslinked tetrapeptide | | |
| (A2pm->D-ala)) (middle of chain) | | |
| u3aga[c]--UDP-3-O-(3-hydroxytetradecanoyl)-N-acetylglucosamine | 1 | 2 |
| 2odecg3p[p]--2-octadecanoyl-sn-glycerol 3-phosphate | 1 | 1 |
| ac[e]--Acetate | 1 | 2 |
| 2odecg3p[c]--2-octadecanoyl-sn-glycerol 3-phosphate | 1 | 1 |
| nad[c]--Nicotinamide adenine dinucleotide | 32 | 33 |
| sucglu[c]--N2-Succinyl-L-glutamate | 1 | 1 |
| cytd[e]--Cytidine | 1 | 2 |
| g3pe[e]--sn-Glycero-3-phosphoethanolamine | 1 | 2 |
| 2agpe180[p]--2-Acyl-sn-glycero-3-phosphoethanolamine (n-C18:0) | 1 | 1 |
| 1agpe160[p]--1-Acyl-sn-glycero-3-phosphoethanolamine (n-C16:0) | 1 | 1 |
| 2agpg120[p]--2-Acyl-sn-glycero-3-phosphoglycerol (n-C12:0) | 1 | 1 |
| 2agpg161[p]--2-Acyl-sn-glycero-3-phosphoglycerol (n-C16:1) | 1 | 1 |
| ade[p]--Adenine | 2 | 2 |
| adele]--Adenine | 1 | 2 |
| cys__L[c]--L-Cysteine | 2 | 7 |
| prbatp[c]--1-(5-Phosphoribosyl)-ATP | 1 | 1 |
| pgp160[c]--Phosphatidylglycerophosphate (dihexadecanoyl; n-C16:0) | 1 | 3 |
| pg180[p]--Phosphatidylglycerol (dioctadecanoyl; n-C18:0) | 2 | 2 |
| pgp160[p]--Phosphatidylglycerophosphate (dihexadecanoyl; n-C16:0) | 1 | 1 |
| 2agpg140[p]--2-Acyl-sn-glycero-3-phosphoglycerol (n-C14:0) | 1 | 1 |
| kdo8p[c]--3-Deoxy-D-manno-octulosonate 8-phosphate | 1 | 1 |
| ala__D[p]--D-Alanine | 7 | 2 |
| ala__D[e]--D-Alanine | 1 | 2 |
| acald[e]--acetaldehyde | 1 | 2 |
| pa120[p]--1;2-didodecanoyl-sn-glycerol 3-phosphate | 1 | 2 |
| 3hcvac11eACP[c]--(R)-3-hydroxy-cis-vacc-11-enoyl-[acyl-carrier protein] | 1 | 1 |
| acald[p]--acetaldehyde | 2 | 2 |
| pa120[c]--1,2-didodecanoyl-sn-glycerol 3-phosphate | 3 | 3 |
| pyr[p]--pyruvate | 2 | 2 |
| 6pgc[c]--6-Phospho-D-gluconate | 1 | 2 |
| pyr[e]--pyruvate | 1 | 2 |
| cdpdodecg[c]--CDP-1;2-dioctadecanoylglycerol | 2 | 3 |
| 1agpg141[p]--1-Acyl-sn-glycero-3-phosphoglycerol (n-C14:1) | 1 | 1 |
| 2agpe120[p]--2-Acyl-sn-glycero-3-phosphoethanolamine (n-C12:0) | 1 | 1 |
| pe140[p]--phosphatidylethanolamine (ditetradecanoyl; n-C14:0) | 1 | 4 |
| 3hcpalm9eACP[c]--(R)-3-hydroxy-cis-palm-9-eoyl-[acyl-carrier protein] | 1 | 1 |
| acser[e]--O-Acetyl-L-serine | 1 | 2 |
| pa160[p]--1;2-dihexadecanoyl-sn-glycerol 3-phosphate | 1 | 2 |
| acser[p]--O-Acetyl-L-serine | 2 | 1 |
| pa160[c]--1;2-dihexadecanoyl-sn-glycerol 3-phosphate | 3 | 3 |
| udcpdp[c]--Undecaprenyl diphosphate | 2 | 1 |
| pgp141[p]--Phosphatidylglycerophosphate (ditetradec-7-enoyl; n-C14:1) | 1 | 1 |
| 2agpe181[p]--2-Acyl-sn-glycero-3-phosphoethanolamine (n-C18:1) | 1 | 1 |
| pgp141[c]--Phosphatidylglycerophosphate (ditetradec-7-enoyl; n-C14:1) | 1 | 3 |
| cdpdhdec9eg[c]--CDP-1;2-dihexadec-9-enoylglycerol | 2 | 3 |
| pg120[p]--Phosphatidylglycerol (didodecanoyl; n-C12:0) | 2 | 2 |
| 2odec11eg3p[p]--2-octadec-11-enoyl-sn-glycerol 3-phosphate | 1 | 1 |
| 1agpg160[p]--1-Acyl-sn-glycero-3-phosphoglycerol (n-C16:0) | 1 | 1 |
| pe160[p]--phosphatidylethanolamine (dihexadecanoyl; n-C16:0) | 1 | 4 |
| 1agpe141[p]--1-Acyl-sn-glycero-3-phosphoethanolamine (n-C14:1) | 1 | 1 |
| 1agpg120[p]--1-Acyl-sn-glycero-3-phosphoglycerol (n-C12:0) | 1 | 1 |
| 4adcho[c]--4-amino-4-deoxychorismate | 1 | 1 |
| pa181[p]--1;2-dioctadec-11-enoyl-sn-glycerol 3-phosphate | 1 | 2 |
| pa181[c]--1;2-dioctadec-11-enoyl-sn-glycerol 3-phosphate | 3 | 3 |
| pg181[p]--Phosphatidylglycerol (dioctadec-11-enoyl; n-C18:1) | 3 | 5 |
| 2agpe161[p]--2-Acyl-sn-glycero-3-phosphoethanolamine (n-C16:1) | 1 | 1 |
| pe120[c]--phosphatidylethanolamine (didodecanoyl; n-C12:0) | 2 | 1 |
| 20dec11eg3p[c]--2-octadec-11-enoyl-sn-glycerol 3-phosphate | 1 | 1 |
| pe120[p]--phosphatidylethanolamine (didodecanoyl; n-C12:0) | 1 | 2 |
| 3hcmrs7eACP[c]--(R)-3-hydroxy-cis-myristol-7-eoyl-[acyl-carrier protein] | 1 | 1 |
| 1agpe120[p]--1-Acyl-sn-glycero-3-phosphoethanolamine (n-C12:0) | 1 | 1 |
| 2kmb[c]--2-keto-4-methylthiobutyrate | 1 | 1 |
| dhpmp[c]--Dihydroneopterin monophosphate | 1 | 1 |
| murein5p5p5p[p]--three linked disacharide pentapeptide murein units | 1 | 1 |
| (uncrosslinked; middle of chain) | | |
| dcmp[c]--dCMP | 2 | 2 |
| gthrd[p]--(reduced) Glutathione | 2 | 1 |
| gthrd[e]--(reduced) Glutathione | 1 | 2 |
| pro__L[p]--L-Proline | 2 | 3 |
| pa140[p]--1;2-ditetradecanoyl-sn-glycerol 3-phosphate | 1 | 2 |
| ser__L[e]--L-Serine | 1 | 2 |
| pa140[c]--1;2-ditetradecanoyl-sn-glycerol 3-phosphate | 3 | 3 |
| pro__L[e]--L-Proline | 1 | 2 |
| ser__L[p]--L-Serine | 2 | 3 |
| ahdt[c]--2-Amino-4-hydroxy-6-(erythro-1;2;3-trihydroxypropyl)dihydropteridine | 1 | 1 |
| triphosphate | | |
| pgp161[c]--Phosphatidylglycerophosphate (dihexadec-9-enoyl; n-C16:1) | 1 | 3 |
| chor[c]--chorismate | 1 | 3 |
| pgp161[p]--Phosphatidylglycerophosphate (dihexadec-9-enoyl; n-C16:1) | 1 | 1 |
| 5mdru1p[c]--5-Methylthio-5-deoxy-D-ribulose 1-phosphate | 1 | 2 |
| glu__L[p]--L-Glutamate | 2 | 4 |
| glu__L[e]--L-Glutamate | 1 | 2 |
| gmhep17bp[c]--D-Glycero-D-manno-heptose 1;7-bisphosphate | 1 | 1 |
| 2agpg160[p]--2-Acyl-sn-glycero-3-phosphoglycerol (n-C16:0) | 1 | 1 |
| arg__L[p]--L-arginine | 4 | 5 |
| arg__L[e]--L-arginine | 1 | 2 |
| co2[e]--CO2 | 1 | 2 |
| anhgm[p]--N-Acetyl-D-glucosamine(anhydrous)N-Acetylmuramic acid | 2 | 1 |
| sucgsa[c]--N2-Succinyl-L-glutamate 5-semialdehyde | 1 | 1 |
| anhgm[e]--N-Acetyl-D-glucosamine(anhydrous)N-Acetylmuramic acid | 1 | 2 |
| hom__L[p]--L-Homoserine | 2 | 1 |
| gln__L[c]--L-Glutamine | 1 | 15 |
| co2[p]--CO2 | 5 | 2 |
| pe161[p]--phosphatidylethanolamine (dihexadec-9enoyl; n-C16:1) | 1 | 4 |
| pser_L[c]--O-Phospho-L-serine | 1 | 1 |
| pe141[c]--phosphatidylethanolamine (ditetradec-7-enoyl; n-C14:1) | 2 | 1 |
| murein5p5p[p]--two linked disacharide pentapeptide murein units (uncrosslinked; | 1 | 3 |
| middle of chain) | | |
| pe141[p]--phosphatidylethanolamine (ditetradec-7-enoyl; n-C14:1) | 1 | 2 |
| 2tdec7eg3p[p]--2-tetradec-7-enoyl-sn-glycerol 3-phosphate | 1 | 1 |
| pgp 140[c]--Phosphatidylglycerophosphate (ditetradecanoyl; n-C14:0) | 1 | 3 |
| pgp140[p]--Phosphatidylglycerophosphate (ditetradecanoyl; n-C14:0) | 1 | 1 |
| hom__L[e]--L-Homoserine | 1 | 2 |
| 2tdec7eg3p[c]--2-tetradec-7-enoyl-sn-glycerol 3-phosphate | 1 | 1 |
| 1agpe180[p]--1-Acyl-sn-glycero-3-phosphoethanolamine (n-C18:0) | 1 | 1 |
| 2obut[c]--2-Oxobutanoate | 1 | 1 |
| cytd[p]--Cytidine | 2 | 3 |
| mn2[c]--Mn2+ | 1 | 1 |
| mn2[p]--Mn2+ | 1 | 1 |
| 2ddg6p[c]--2-Dehydro-3-deoxy-D-gluconate 6-phosphate | 1 | 1 |
| indole[p]--Indole | 2 | 1 |
| indole[e]--Indole | 1 | 2 |
| agm[e]--agmatine | 1 | 2 |
| h2s[e]--Hydrogen sulfide | 1 | 2 |
| h2s[p]--Hydrogen sulfide | 2 | 1 |
| 2ddecg3p[p]--2-dodecanoyl-sn-glycerol 3-phosphate | 1 | 1 |
| 2ddecg3p[c]--2-dodecanoyl-sn-glycerol 3-phosphate | 1 | 1 |
| for[p]--Formate | 2 | 4 |
| agm[p]--agmatine | 3 | 2 |
| hdeACP[c]--cis-hexadec-9-enoyl-[acyl-carrier protein] (n-C16:1) | 4 | 5 |
| h[c]--H+ | 351 | 187 |

TABLE 7
Structurally constrained concentrations for metabolites serving as energy currency.
(h = chloroplast, c = cytosol, m = mitochondria, n = nucleus, p = periplasm, e = external). The
table summarizes the networks in which Eq. (1) holds for NADH, NAD, NADP, NADPH, ATP,
and H+. The table includes the respective compartments in which Eq. (1) can be applied for
the investigated metabolites.

| Network | NADH | NAD | NADP | NADPH | ATP | H+ |
|---|---|---|---|---|---|---|
| A. niger | | c | c | c | c | |
| A. thaliana | | | h | | h, c, m | |
| C. reinhardtii | | | h | | h | h, c |
| E. coli K12 | | c | | | c | c, p |
| H. sapiens | c | c | | c | c, n | c |
| M. acetivorans | | c | | | c | c |
| M. barkeri | | c | | | c | c |
| M. pneumoniae | | | | | | |
| N. pharaonic | | c | | | c | |
| P. putida | | | | | c | c, e |
| T. maritima | | c | c | | c | c, e |
| S. aureus | | c | | | | c, e |
| Synechocystis sp. | | c | | | c | c, p |
| Y. pestis | | c | | | c | c |
| Number of | 1 | 10 | 4 | 2 | 12 | 10 |
| networks where | | | | | | |
| Eq.(1) can be | | | | | | |
| applied | | | | | | |

Altogether, our findings indicate that the concentration ranges for coenzymes and other components essential for fueling metabolism can be established by controlling few ratios of fluxes, despite their involvement in hundreds of reactions. Moreover, they imply that the network architecture may be organized such that the concentrations of these metabolites are intrinsically constrained and easy to control.

### Example 7

### Absolute Concentration Robustness

As a consequence of Eq. (1), if two reactions Rₚ and Rₛ⁻ⁱ are fully coupled, then

$$\frac{{v}_{p}}{{v}_{s}^{- i}}$$

is constant, i.e., the SCC component Xᵢ has in addition the same constant concentration in any positive steady state that the network admits. Since the steady-state concentration of this component does not depend on initial conditions, it exhibits absolute concentration robustness (ACR) (Shinar, G. & Feinberg, M. Structural sources of robustness in biochemical reaction networks. Science 327, 1389-1391, doi: 10.1126/science.1183372 (2010)). We note that even in the absence of values for the rate constants, our approach can be used to specify components with ACR, even though the concentrations in that case cannot be determined.

### Example 8

Metabolite Concentration Data Set of Ishii et al. (Multiple High-Throughput Analyses Monitor the Response of E. coli to Perturbations. Science: 316 (5824): 593-7. Doi: 10.1126/Science. 1132067. PubMed PMID: 17379776 (2007).

We use the measurements of steady-state concentrations of 182 metabolites from E. coli under different growth scenarios [28]. This data set includes 15 of the 199 cytosolic SCC metabolites found in the genome-scale model. We also have access to rates of glucose and oxygen uptakes, carbon dioxide release as well as growth from the same experiments (Ishii et al. (Multiple high-throughput analyses monitor the response of E. coli to perturbations. Science; 316 (5824): 593-7. doi: 10.1126/science.1132067. PubMed PMID: 17379776 (2007)), which we use as constraints to a genome-scale metabolic network of E. coli. It has been shown that E. coli does not optimize a single objective (e.g., growth), but its steady-state flux distributions result from the trade-off between tasks of optimizing growth, ATP synthesis, and total flux (Schuetz, R., Zamboni, N., Zampieri, M., Heinemann, M. & Sauer, U. Multidimensional optimality of microbial metabolism. Science 336, 601-604, doi: 10.1126/science.1216882 (2012)). Since growth rate is fixed from measurements, we optimize the weighted average of ATP synthesis and total flux, with a weighting factor of 0.1 on ATP synthesis to reduce the effect of the order difference in the respective optimum observed when ATP production and total flux are optimized individually. Here, too, at the obtained optimum we can efficiently estimate ranges for the relevant flux ratios. In addition, we compare obtained concentration ranges with those predicted when maximization of ATP is used as the only objective. To obtain estimates for

$$\frac{{σ}_{p}}{{σ}_{s}^{- i}} ,$$

we use three replicates for the concentration data and predictions of ranges for relevant flux ratios at growth rate of 0.2 h⁻¹. Formula (3) can then be applied to determine concentration ranges based on

$$\frac{{σ}_{p}}{{σ}_{s}^{- i}}$$

for a combination of replicales, to investigate the effect of outliers. We predict in turn the concentration ranges for three other growth rates (i.e., 0.4, 0.5, and 0.7 h⁻¹).

For the objective of optimizing ATP synthesis and total flux, our results demonstrate that measurements for 9, 10, and 6 of the 15 SCC metabolites fall in the predicted concentration range for the three growth rates, respectively (FIG. 10). Nevertheless, the Spearman correlation between the measured values and the predicted lower and upper bounds is significant and larger than 0.57 and 0.56, respectively. Therefore, the approach can be used to compare the ordering of lower or upper bounds between different experimental scenarios. In addition, this analysis highlights the effect of the replicates of metabolite concentrations used in calculating the values of

$$\frac{{σ}_{p}}{{σ}_{s}^{- i}} ,$$

since estimates for some of the replicates may be outliers (FIG. 10). In contrast, we find that 4, 5 and 2 of the 15 SCC metabolites fall in the measured range for the three growth rates when maximization of ATP is used as objective. Moreover, we cannot predict concentrations for 8 out of the 15 SCC metabolites due to numerical instabilities arising when using this objective under the additionally imposed constraints on growth. The reasons for the discrepancy between the predicted and measured values under both objectives include the combination of at least three factors: the inability to distinguish the concentrations of free metabolites from those bound to macromolecules experimentally (Bennett B D, Kimball E H, Gao M, Osterhout R, Van Dien S J. Rabinowitz J D. Absolute metabolite concentrations and implied enzyme active site occupancy in Escherichia coli. Nature chemical biology; 5 (8): 593-9. doi: 10.1038/nchembio. 186. PubMed PMID: 19561621; PubMed Central PMCID: PMCPMC2754216 (2009)), model (and objective) inaccuracies, and the simplifying assumption of mass action kinetic. Nevertheless, the approach can be extended to consider networks with kinetic laws derived from mass action which involve enzyme forms (e.g., Michaelis-Menten) at cost of increased data requirements for application.

### Example 9

Metabolite Concentration Data Set of Gerosa et al. (Pseudo-Transition Analysis Identifies the Key Regulators of Dynamic Metabolic Adaptations from Steady-State Data. Cell Systems; 1 (4): 270-82. doi: 10.1016/j.cels.2015.09.008. PubMed PMID: 27136056 (2015))

We use the measurements of steady-state concentrations of 43 metabolites from E. coli grown in eight different carbon sources (Gerosa et al., Pseudo-transition Analysis Identifies the Key Regulators of Dynamic Metabolic Adaptations from Steady-State Data. Cell systems; 1 (4): 270-82. doi: 10.1016/j.cels.2015.09.008. PubMed PMID: 27136056 (2015)) [32]. This data set includes ten of the 199 cytosolic SCC metabolites found in the genome-scale model. We also have access to rates of carbon uptake, some secretion rates, as well as growth from the same experiments, which we use as constraints to a genome-scale metabolic network of E. coli. Since growth rate is fixed from measurements, as above, we optimize the weighted average of ATP synthesis and total flux, with weighting factors 0.001 for ATP synthesis and 1000 for total flux to reduce the effect of the order difference and make the comparison to optimization of ATP synthesis. Different weighting factors are used in comparison to the analysis of the data set from Ishii et al., above, since different constraints are used that affect the optimal values of the individual objectives. Here, too, at the obtained optimum we can efficiently estimate ranges for the relevant flux ratios. To obtain estimates for

$$\frac{{σ}_{p}}{{σ}_{s}^{- i}} ,$$

we use the metabolite concentrations from growth on acetate. We then predict the concentration ranges for the ten SCC metabolites for the seven other carbon sources.

In case of succinate as only carbon source we obtain a model with no feasible solution, so no concentrations could be predicted for that case without further model adaptations. In the remaining growth conditions, depending on the objective and growth condition analyzed, three to five predictions of concentrations resulted in minimum values larger than the respective maximum (missing black bars). This observation is a result of numerical instabilities occurring if flux values vₚ and vₛ⁻ⁱ in Formula (1) differ by several orders of magnitude. The Spearman correlation between the average measured and predicted concentrations (FIG. 11) when optimizing ATP synthesis is 0.63 (p-value 3*10⁻⁴), while it is only 0.33 (p-value 0.03) when ATP synthesis and total flux are optimized. In addition, the Spearman correlation between the measured and predicted upper and lower bounds when maximization of ATP is used results in higher correlation values (upper bounds 0.61 (p-value 4.3*10⁻⁴), lower bounds 0.85 (p-value 5.9*10⁻⁹)) than those when optimization of ATP synthesis and total flux are employed (upper bounds 0.21 (p-value 0.17), lower bounds 0.54 (p-value 1.6*10⁻⁴)). These findings imply that the usage of different objectives to estimate flux ratios and through them concentrations of metabolites can also be used to discern importance of optimized objectives in a particular experiment.

### Example 10

### Changes in Metabolite Concentrations in Knock-Out Mutants

The fully parameterized kinetic model of E. coli can be used to test the applicability of the approach to predict changes in metabolite concentrations in metabolic engineering scenarios. Here, we test the performance of the approach with knock-out mutants based on the following procedure: We make use of the model parameterization to simulate a steady-state concentration and flux distribution from initial physiologically reasonable values for metabolite concentrations. The resulting steady-state concentrations and fluxes yield a wild type reference. We then knock-out each reaction and predict positive steady state flux distribution closest to the wild type reference, following the Minimization of Metabolic Adjustment (MOMA) approach (Segre D, Vitkup D, Church G M. Analysis of optimality in natural and perturbed metabolic networks. Proceedings of the National Academy of Sciences of the United States of America; 99 (23): 15112-7. doi: 10.1073/pnas.232349399. PubMed PMID: 12415116; PubMed Central PMCID: PMCPMC137552 (2002)). The resulting flux distribution is used to calculate the concentrations of the 23 SCC metabolites following our approach (Formula (1)). In the last step, the predicted changes in concentration of the SCC metabolites with respect to the reference are compared to the changes from kinetic simulations of the knock-out with the wild-type reference specifying the initial conditions. We observe similar ranges for the predicted and simulated fold-changes in SCC concentration over all 23 SCC metabolites and knock-outs of 929 reactions for which we were able to simulate a steady-state knock-out flux distribution (FIG. 12). We grouped the fold-changes into 12 bins, given in the x-axis of FIG. 12. For ten SCC metabolites, the predicted fold change of at least 29% of the knock-outs is in the same bin as the simulated fold change. The highest overlaps are observed for AMP (39%), phosphoenolpyruvate (38%) and isocitrate (37%). In contrast, the fold changes in concentration for metabolites like succinyl-CoA, acetyl-CoA, oxaloacetate, malate and pyruvate are in the same class as simulated for at most 1% of the knock-outs. The lack of correspondence between simulated and predicted concentrations for some SCC metabolites indicates that principles others than those used in MOMA shape the metabolic adjustment of knock-out mutants. In contrast to our findings, application of the art-established method of thermodynamics-based flux analysis (TMFA) resulted in unconstrained ranges for concentrations; therefore, no correlation between upper/lower simulated and predicted bounds could be observed.

1. A computer-implemented method of calculating
(a) the ranges of the concentration xᵢ of a component Xᵢ;
(b) the flux(es) and/or flux ratio(s) which determine the concentration ranges of the said component Xᵢ; and/or
(c) the reaction rate constant(s) and/or their ratio(s) which determine the concentration ranges of said component Xᵢ in a network of chemical reactions defined by a stoichiometric matrix N;
said calculating comprising evaluating formula (1):
$$min \{ Q , F , {P}_{j} \} {λ}_{i} \leq {x}_{i} \leq max \{ Q , F , {P}_{j} \} {λ}_{i} \qquad ( 1 )$$
wherein
(i) N is N⁺-N⁻;
N⁺ is the matrix defining the stoichiometry of products of each reaction in said network;
N⁻ is the matrix defining the stoichiometry of substrates of each reaction in said network;
Sⱼ is the set of reactions in said network which have a component Xⱼ as one of their substrates;
F is a set of steady-state fluxes; and
Pⱼ is the set of reactions in said network which have component Xⱼ as one of their products;
$${λ}_{i} = \frac{{σ}_{p}}{{σ}_{s}^{- i}} \frac{{v}_{p}}{{v}_{s}^{- i}} ; \qquad ( ii )$$
vₚ is the flux of reaction Rₚ∈Pⱼ, Rₚ having Xⱼ as one of its products;
vₛ⁻ⁱ is the flux of reaction Rₛ⁻ⁱ∈Q, Rₛ⁻ⁱ differing from reaction Rₛ in that one substrate molecule of Xᵢ is missing in comparison to reaction Rₛ;
$${σ}_{p} = {\sum }_{k ϵ Pj} {N}_{jk}^{+} \frac{{v}_{k}}{{v}_{p}} ;$$
$${σ}_{s}^{- i} = {\sum }_{l ϵ Sj} {N}_{jl}^{-} \frac{{θ}_{l}}{{θ}_{l}^{- i}} \frac{{v}_{l}^{- i}}{{v}_{s}^{- i}} ;$$
θₗ is the reaction rate constant for reaction Rₗ;
θₗ⁻ⁱ is the reaction rate constant for reaction Rₗ⁻ⁱ;
(iii) Q is a subset of Sⱼ⁻ⁱ such that for every reaction Rₗ∈Sⱼ there is one reaction Rₗ⁻ⁱ∈Q; and
Sⱼ⁻ⁱ is a set of chemical reactions differing from the set of reactions Sⱼ in that one substrate molecule of Xᵢ is missing in comparison to a reaction R in Sⱼ.

2. The method of claim 1, wherein
(a) concentrations and/or ranges thereof are determined using fluxes and reaction rate constants as input;
(b) fluxes, flux ratios and/or ranges thereof are determined using concentrations and reaction rate constants as input; or
(c) reaction rate constants, their ratios and/or ranges thereof are determined using concentrations and fluxes as input.

3. The method of claim 2, wherein
(a) up to 10%, up to 20%, up to 30%, up to 40%, up to 50%, up to 60%, up to 70% or up to 80% of said input is estimated;
(b) experimentally determined input does not require experiments involving isotope labelling; and/or
(c) concentrations, to the extent they are used as input, are determined by means of mass spectrometry.

4. The method of claim 1, wherein said method is applied two or more times, and wherein each time more input is provided, the additional input being derived from one or more previous runs.

5. The method of claim 1, said method comprising applying said method
(a) to at least two networks with different architectures, said different architectures being defined in terms of different stoichiometric matrices N; and/or
(b) to the same network using at least two different inputs.

6. The method of claim 1, furthermore comprising identifying for a given network and given inputs those components for which the ratio defined by formula (2)
$$\frac{max \{ Q , F , {P}_{j} \} {x}_{i}}{min \{ Q , F , {P}_{j} \} {x}_{i}} \qquad ( 2 )$$
is less than 2.0, less than 1.5, less than 1.2, less than 1.1, less than 1.05 or equal to 1.0, thereby identifying components exhibiting absolute concentration robustness (ACR).

7. The method of claim 6,
(a) if said method comprises applying said method to at least two networks with different architectures, said different architectures being defined in terms of different stoichiometric matrices N, furthermore comprising identifying those components which exhibit ACR in a first given network, but not in a second given network, thereby identifying a marker which allows to distinguish said first network from said second network; or
(b) if said method comprises applying said method to the same network using at least two different inputs, further comprising identifying those components which exhibit ACR for a first given input, but not for a second given input, thereby identifying a marker which allows to distinguish a first state of said network from a second state of said network.

8. The method of claim 5, further comprising determining concentration range or mean concentration for Xᵢ in each network or for each input, wherein statistically significant differences between the concentration ranges or mean concentrations for a first given network and a second given network are indicative of component Xᵢ being a marker which allows to distinguish a first network from a second network, respectively, and statistically significant differences between the concentration ranges or mean concentrations for a first given input and a second given input are indicative of component Xᵢ being a marker which allows to distinguish a first network state from a second network state.

9. The method of claim 7, wherein said first network or network state is a healthy organism or cell or a healthy state of an organism or cell, respectively, and said second network or network state is a diseased organism or cell or a diseased state of an organism or cell, respectively.

10. The method of claim 1, wherein said network
(a) is cell-wide, organism-wide, cell-free or chemical network;
(b) comprises more than 50, more than 100, or more than 200 reactions; and/or
(c) comprises second and/or higher order reactions.

11. The method of claim 1, wherein said component is selected from proteins, polypeptides, nucleic acids, lipids, carbohydrates, small organic molecules, metabolites and any combination thereof.

12. A method of calculating the ranges of the concentration xᵢ of a component Xᵢ, the flux(es) and/or flux ratio(s) which determine the concentration ranges of the said component Xᵢ, and/or the reaction rate constant(s) and/or their ratio(s) which determine the concentration ranges of said component Xᵢ in a network of chemical reactions R defined by a stoichiometric matrix N, the method comprising using the formula (1) as defined in claim 1 for said calculating.

13. A computer program comprising instructions to cause a computer to execute the steps of the method of claim 1.

14. A computer-readable medium comprising instructions which, when executed on a computer, cause said computer to execute the steps of the method of claim 1.

15. A computer-readable medium having stored thereon the computer program of claim 13.
